(19) United States
(12) Patent Application Publication
(10) Pub. No.: US 2024/0165135 A1
(43) Pub. Date: May 23, 2024

(54) COMPOSITIONS AND METHODS FOR THE TREATMENT OF CANCER

(71) Applicant: The State of Israel, Ministry of Agriculture & Rural Development, Agricultural Research Organization, Rishon-LeZion (IL)

(72) Inventors: Hinanit KOLTAI, Rishon-LeZion (IL); Nurit SHALEV, Tirat Yehuda (IL); Hadar PEERI, Karmei Yosef (IL)

(73) Assignee: The State of Israel, Ministry of Agriculture & Rural Development, Agricultural Research Organization, Rishon-LeZion (IL)

(21) Appl. No.: 18/376,498

(22) Filed: Oct. 4, 2023

Related U.S. Application Data
(63) Continuation of application No. PCT/IL2022/050357, filed on Apr. 5, 2022.
(60) Provisional application No. 63/287,149, filed on Dec. 8, 2021.
(60) Provisional application No. 63/234,340, filed on Aug. 18, 2021.
(60) Provisional application No. 63/170,604, filed on Apr. 5, 2021.

(51) Int. Cl. A61K 31/00; A61K 31/454; A61K 33/243; A61K 36/185; A61P 35/00

(52) U.S. Cl. CPC A61K 31/658; A61K 31/454; A61K 33/243; A61K 36/185; A61P 35/00

(57) ABSTRACT

Compositions capable of reducing viability, inducing cell cycle arrest and/or reducing proliferation and/or migration of Glioblastoma multiforme (GMB) and ovarian cancer cells and methods of use thereof are provided. The compositions and uses thereof comprise phytocannabinoids.

### RELATED APPLICATIONS

10 This application is a Continuation of PCT Patent Application No. PCT/IL2022/050357 having International filing date of Apr. 5, 2022, which claims the benefit of priority under 35 USC § 119(e) of U.S. Provisional Patent Application Nos. 63/287,149 filed on Dec. 8, 2021, 63/234,340 filed on Aug. 18, 2021 and 63/170,604 filed on Apr. 5, 2021. The contents of the above applications are all incorporated by reference as if fully set forth herein in their entirety.

### FIELD AND BACKGROUND OF THE INVENTION

The present invention, in some embodiments thereof, relates to compositions and methods for the treatment of cancer.

Glioma are primary brain tumors that arise from glial cells and account for ˜80% of all malignant brain tumors [1]. Among brain tumors, glioblastoma multiforme (GBM, WHO grade IV) is the most frequent, aggressive and lethal subtype [1, 2]. These tumors are characterized by high cellular proliferation and angiogenesis resulting in rapid tumor growth and, consequently, necrosis. GBM cells also exhibit high migration and invasive properties, which allow them to produce metachronous lesions and even to spread through the brain parenchyma. Furthermore, GBM tumors contain a subpopulation of glioma stem-like cells (GSCs), which, at least partially, account for the high resistance to therapy and recurrence rates of these tumors. Standard GBM therapies include maximal surgical resection followed by radio- and chemotherapy [3]. Yet, in recurrent or progressive GBM no standard of care is established and treatments include surgery, re-irradiation, combined modality therapy, systemic therapies and supportive care [3].

Ovarian cancer (OC) is the second most common and the most lethal gynecologic malignancy in the western world; about 70% of cases are diagnosed at an advanced stage. Late-stage ovarian cancer is incurable in the majority of cases. The estimate for 2021 is of 21,410 new cases of OC diagnosed and 13,770 OC deaths in the United States. Epithelial OC typically presents in postmenopausal women, with a few months of abdominal pain and distension, vague and subtle symptoms that are often dismissed. Many women are under diagnosed for at least 6 months. Also, there is no screening test for OC and population level monitoring does not reduce mortality. When finally diagnosed, most women have advanced disease, for which the standard of care remains surgery and platinum-based cytotoxic chemotherapy. In about 80% of the cases disease relapse is expected, on average after 24 months, and ultimately multi-drug resistance develops, with very few women surviving five years after diagnosis.

Marijuana (Cannabis sativa) contains more than 500 constituents, among them phytocannabinoids, terpenes and flavonoid [ElSohly et al., Phytochemistry of Cannabis sativa L. Phytocannabinoids, Springer (2017) 1-36]. An increasing number of studies have shown that phytocannabinoids can prevent proliferation, metastasis, angiogenesis and induce apoptosis in a variety of cancer cell types including breast, lung, prostate, skin, intestine, glioma, and others (e.g. International Patent Application Publication Nos: WO2016/097831, WO2018/163163, WO2018/163164 and WO2020/121312, EP Patent No: EP1071417, U.S. patent Nos: U.S. Pat. Nos. 8,632,825 and 8,632,825).

Phytocannabinoids such as Δ9-tetrahydrocannabinol (THC), cannabidiol (CBD) and Cannabigerol (CBG), were shown to effect growth, viability and invasion of GBM cells via different mechanisms, including e.g. cell cycle arrest, oxidative stress, endoplasmic reticulum (ER)-stress, autophagy and apoptosis [Ellert-Miklaszewska, A et al. Glioma Signaling 2020, 223-241; Dumitru, C. et al. Front. Mol. Neurosci. 2018, 11, 159; Hernández-Tiedra, S. et al. Autophagy 2016, 12, 2213-2229; Salazar, M. et al. J. Clin. Invest. 2009, 119, 1359-1372; Luís, Â. et al., Eur. J. Pharmacol. 2020, 876; Galanti, G. Acta Oncol. 2008, 47, 1062-1070; Solinas, M. et al. PLoS One 2013, 8, e76918; Lah, T. et al. Cells 2021, 10, 340; Hart, S. et al. Cancer Res. 2004, 64, 1943-1950; Salazar, M. et al. Autophagy 2009, 5, 1048-1049; Marcu, J. P. et al. Mol. Cancer Ther. 2010, 9, 180-189]. Moreover, promising clinical evidence suggests effective cannabinoid-based treatments against GBM [Dumitru, C. et al. Front. Mol. Neurosci. 2018, 11, 159; Guzman, M. et al. Br. J. Cancer 2006, 95, 197-203; Torres, S.; Lorente, M.; Rodríguez-Fornés, F.; Hernández-Tiedra, S.; Salazar, M.; García-Taboada, E.; Barcia, J.; Guzmán, M.; Velasco, G. A combined preclinical therapy of cannabinoids and temozolomide against glioma. Mol. Cancer Ther. 2011, 10, 90-103; Schultz, S.; Beyer, M. GW Pharmaceuticals Achieves Positive Results in Phase 2 Proof of Concept Study in Glioma. 2017. Available online: www(dot)ir(dot)gwpharm(dot)com/static-files/cde942fe-555c-4b2f-9cc9-f34d24c7ad2 (accessed on 1 Feb. 2021).

### SUMMARY OF THE INVENTION

According to an aspect of some embodiments of the present invention there is provided a method of treating cancer in a subject in need thereof, the method comprising administering to the subject a therapeutically effective amount of a composition selected from the group consisting of:
(i) a composition comprising phytocannabinoids, wherein the phytocannabinoids comprise at least 70% cannabigerol (CBG) and at least one phytocannabinoid selected from the group consisting of cannabinol (CBN) and tetrahydrocannabivarin (THCV);
(ii) a composition comprising phytocannabinoids, wherein the phytocannabinoids comprise at least 85% tetrahydrocannabinol (THC) and at least 1.5% cannabigerol (CBG);
(iii) a composition comprising phytocannabinoids, wherein the phytocannabinoids comprise at least 50% tetrahydrocannabinol (THC), at least 10% cannabichromene (CBC) and at least one phytocannabinoid selected from the group consisting of cannabidivarin (CBDV), cannabidivarinic acid (CBDVA), cannabigerol (CBG), cannabinol (CBN) and tetrahydrocannabinolic acid (THCA); and
(iv) a composition comprising phytocannabinoids, wherein the phytocannabinoids comprise at least 80% tetrahydrocannabinol (THC) and at least three phytocannabinoids selected from the group consisting of cannabichromene (CBC), cannabigerol (CBG), cannabinol (CBN), tetrahydrocannabinolic acid (THCA) and tetrahydrocannabivarin (THCV),
thereby treating the cancer in the subject.

According to an aspect of some embodiments of the present invention there is provided a composition selected form the group consisting of:
(i) a composition comprising phytocannabinoids, wherein the phytocannabinoids comprise at least 70% cannabigerol (CBG) and at least one phytocannabinoid selected from the group consisting of cannabinol (CBN) and tetrahydrocannabivarin (THCV);
(ii) a composition comprising phytocannabinoids, wherein the phytocannabinoids comprise at least 85% tetrahydrocannabinol (THC) and at least 1.5% cannabigerol (CBG);
(iii) a composition comprising phytocannabinoids, wherein the phytocannabinoids comprise at least 50% tetrahydrocannabinol (THC), at least 10% cannabichromene (CBC) and at least one phytocannabinoid selected from the group consisting of cannabidivarin (CBDV), cannabidivarinic acid (CBDVA), cannabigerol (CBG), cannabinol (CBN) and tetrahydrocannabinolic acid (THCA); and
(iv) a composition comprising phytocannabinoids, wherein the phytocannabinoids comprise at least 80% tetrahydrocannabinol (THC) and at least three phytocannabinoids selected from the group consisting of cannabichromene (CBC), cannabigerol (CBG), cannabinol (CBN), tetrahydrocannabinolic acid (THCA) and tetrahydrocannabivarin (THCV),
for use in treating cancer in a subject in need thereof.

According to an aspect of some embodiments of the present invention there is provided a method of reducing viability, inducing cell cycle arrest and/or reducing proliferation and/or migration of a cancerous cell, the method comprising contacting the cancerous cell with a composition selected from the group consisting of:
(i) a composition comprising phytocannabinoids, wherein the phytocannabinoids comprise at least 70% cannabigerol (CBG) and at least one phytocannabinoid selected from the group consisting of cannabinol (CBN) and tetrahydrocannabivarin (THCV);
(ii) a composition comprising phytocannabinoids, wherein the phytocannabinoids comprise at least 85% tetrahydrocannabinol (THC) and at least 1.5% cannabigerol (CBG);
(iii) a composition comprising phytocannabinoids, wherein the phytocannabinoids comprise at least 50% tetrahydrocannabinol (THC), at least 10% cannabichromene (CBC) and at least one phytocannabinoid selected from the group consisting of cannabidivarin (CBDV), cannabidivarinic acid (CBDVA), cannabigerol (CBG), cannabinol (CBN) and tetrahydrocannabinolic acid (THCA); and
(iv) a composition comprising phytocannabinoids, wherein the phytocannabinoids comprise at least 80% tetrahydrocannabinol (THC) and at least three phytocannabinoids selected from the group consisting of cannabichromene (CBC), cannabigerol (CBG), cannabinol (CBN), tetrahydrocannabinolic acid (THCA) and tetrahydrocannabivarin (THCV).

According to an aspect of some embodiments of the present invention there is provided a composition selected from the group consisting of:
(i) a composition comprising the phytocannabinoids listed in the F4 composition of Table 1 in percentages as listed in the F4 composition of Table 1±10%;
(ii) a composition comprising the phytocannabinoids listed in the F5 composition of Table 1 in percentages as listed in the F5 composition of Table 1±10% or a composition comprising the phytocannabinoids listed in the F6 composition of Table 1 in percentages as listed in the F6 composition of Table 1±10%;
(iii) a composition comprising the phytocannabinoids listed in the F7 composition of Table 1 in percentages as listed in the F7 composition of Table 1±10%;
(iv) a composition comprising the phytocannabinoids listed in the crude extract composition of Table 1 in percentages as listed in the crude extract composition of Table 1±10%;
(v) a composition comprising the phytocannabinoids listed in the sCBD crude extract composition of Table 7 in percentages as listed in the crude extract composition of Table 7±10%; and
(vi) a composition comprising the phytocannabinoids listed in the PARIS crude extract composition of Table 7 in percentages as listed in the crude extract composition of Table 7±10%.

According to an aspect of some embodiments of the present invention there is provided a method of treating cancer in a subject in need thereof, the method comprising administering to the subject a therapeutically effective amount of the composition, thereby treating the cancer in the subject.

According to an aspect of some embodiments of the present invention there is provided the composition, for use in treating cancer in a subject in need thereof.

According to an aspect of some embodiments of the present invention there is provided a method of reducing viability, inducing cell cycle arrest and/or reducing proliferation and/or migration of a cancerous cell, the method comprising contacting the cancerous cell with the composition.

According to some embodiments of the invention, the contacting is effected ex-vivo or in-vitro.

According to some embodiments of the invention, the contacting is effected in-vivo.

According to some embodiments of the invention, the method further comprises administering to the subject a therapeutically effective amount of an anti-cancer agent.

According to some embodiments of the invention, the composition further comprises an anti-cancer agent.

According to some embodiments of the invention, the method further comprises contacting the cancerous cell with an anti-cancer agent.

According to an aspect of some embodiments of the present invention there is provided an article of manufacture comprising an anti-cancer agent and a composition selected form the group consisting of:
(i) a composition comprising phytocannabinoids, wherein the phytocannabinoids comprise at least 70% cannabigerol (CBG) and at least one phytocannabinoid selected from the group consisting of cannabinol (CBN) and tetrahydrocannabivarin (THCV);
(ii) a composition comprising phytocannabinoids, wherein the phytocannabinoids comprise at least 85% tetrahydrocannabinol (THC) and at least 1.5% cannabigerol (CBG);
(iii) a composition comprising phytocannabinoids, wherein the phytocannabinoids comprise at least 50% tetrahydrocannabinol (THC), at least 10% cannabichromene (CBC) and at least one phytocannabinoid selected from the group consisting of cannabidivarin (CBDV), cannabidivarinic acid (CBDVA), cannabigerol (CBG), cannabinol (CBN) and tetrahydrocannabinolic acid (THCA); and
(iv) a composition comprising phytocannabinoids, wherein the phytocannabinoids comprise at least 80% tetrahydrocannabinol (THC) and at least three phytocannabinoids selected from the group consisting of cannabichromene (CBC), cannabigerol (CBG), cannabinol (CBN), tetrahydrocannabinolic acid (THCA) and tetrahydrocannabivarin (THCV).

According to an aspect of some embodiments of the present invention there is provided an article of manufacture comprising an anti-cancer agent and the composition.

According to some embodiments of the invention, the anti-cancer agent and the composition are provided in a co-formulation.

According to some embodiments of the invention, the anti-cancer agent and the composition are provided in separate formulations.

According to some embodiments of the invention, the anti-cancer agent comprises a chemotherapy.

According to some embodiments of the invention, the chemotherapy is selected from the group consisting of cisplatin and niraparib.

According to some embodiments of the invention, the composition has anti-cancer activity on Glioblastoma multiforme (GMB) cells and/or ovarian cancer cells.

According to some embodiments of the invention, the composition has a combined synergistic anti-cancer activity on glioblastoma multiforme (GMB) cells and/or ovarian cancer cells as compared to each of the phytocannabinoids comprised in the composition when administered as a single agent.

According to some embodiments of the invention, the anti-cancer activity is manifested by reduced viability, cell cycle arrest and/or reduced proliferation and/or migration.

According to some embodiments of the invention, the anti-cancer activity is manifested by expression of genes associated with ER stress, reduced viability of glioma stem-like cells (GSCs), reduced motility and invasion, disintegration of F-actin and/or reduced colony/neurosphere formation, cell cycle distribution, cell killing, MAPK4 signaling.

According to some embodiments of the invention, the cancer is glioblastoma.

According to some embodiments of the invention, the cancer is Glioblastoma multiforme (GMB).

According to some embodiments of the invention, the cancer is ovarian cancer.

According to some embodiments of the invention, the composition (i) further comprises cannabidiol (CBD).

According to some embodiments of the invention, the composition (i) comprises all of the phytocannabinoids.

According to some embodiments of the invention, the composition (i) comprises at least 2% of the CBN and/or at least 5% of the THCV.

According to some embodiments of the invention, the composition (i) comprises at least 4% of the CBD.

According to some embodiments of the invention, the composition (i) comprises no more than 10% of the CBD.

According to some embodiments of the invention, the composition (i) is devoid of tetrahydrocannabinol (THC).

According to some embodiments of the invention, the composition (i) is devoid of tetrahydrocannabinolic acid (THCA), cannabigerolic acid (CBGA), cannabidivarinic acid (CBDVA), cannabidivarin (CBDV) and/or cannabichromene (CBC).

According to some embodiments of the invention, the composition (i) comprises at least 75% of the CBG.

According to some embodiments of the invention, a concentration ratio of the CBG, the CBN, the THCV and/or the CBD in the composition (i) is 1 CBG:0.01-0.05 CBN:0.05-0.3 THCV:0.06-0.09 CBD.

According to some embodiments of the invention, the composition (i) comprises the phytocannabinoids listed in the F4 composition of Table 1.

According to some embodiments of the invention, the composition (i) comprises percentages of phytocannabinoids as listed in the F4 composition of Table 1±10%.

According to some embodiments of the invention, the composition (ii) further comprises cannabinol (CBN).

According to some embodiments of the invention, the composition (ii) comprises less than 10% of the CBG and/or the CBN.

According to some embodiments of the invention, the composition (ii) comprises less than 5% of the CBG and/or the CBN.

According to some embodiments of the invention, rein the composition (ii) comprises at least 90% of the THC.

According to some embodiments of the invention, the composition (ii) comprises at least 95% of the THC.

According to some embodiments of the invention, the composition (ii) is devoid of cannabidiol (CBD), cannabidivarin (CBDV) and/or cannabidivarinic acid (CBDVA).

According to some embodiments of the invention, the composition (ii) is devoid of cannabichromene (CBC), cannabigerolic acid (CBGA), tetrahydrocannabinolic acid (THCA) and/or THCV.

According to some embodiments of the invention, a concentration ratio of the THC, the CBG and/or the CBN in the composition (ii) is 1 THC:0.03-0.05 CBG:0.04-0.06 CBN.

According to some embodiments of the invention, the composition (ii) comprises the phytocannabinoids listed in the F5 composition of Table 1.

According to some embodiments of the invention, the composition (ii) comprises percentages of phytocannabinoids as listed in the F5 composition of Table 1±10%.

According to some embodiments of the invention, a concentration ratio of the THC, the CBG and/or the CBN in the composition (ii) is 1 THC:0.01-0.03 CBG:0.007-0.015 CBN.

According to some embodiments of the invention, the composition (ii) comprises the phytocannabinoids listed in the F6 composition of Table 1.

According to some embodiments of the invention, the composition (ii) comprises percentages of phytocannabinoids as listed in the F5 composition of Table 1±10%.

According to some embodiments of the invention, the at least one in the composition (iii) comprises at least two.

According to some embodiments of the invention, the composition (iii) comprises all of the phytocannabinoids.

According to some embodiments of the invention, the composition (iii) comprises less than 10% of each of the CBDV, the CBDVA, the CBG the CBN and/or the THCA.

According to some embodiments of the invention, the composition (iii) comprises less than 5% of each of the CBDV, the CBDVA, the CBG the cannabinol (CBN) and/or the THCA.

According to some embodiments of the invention, the composition (iii) comprises at least 60% of the THC.

According to some embodiments of the invention, the composition (iii) is devoid of cannabidiol (CBD), cannabigerolic acid (CBGA) and/or tetrahydrocannabivarin (THCV).

According to some embodiments of the invention, a concentration ratio of the THC, the CBC, the CBDV, the CBDVA, the CBG, the CBN and/or the THCA in the composition (iii) is 1 THC:0.2-0.5 CBC:0.01-0.04 CBDV, 0.005-0.025 CBDVA:0.01-0.03 CBG:0.015-0.04 CBN:0.015-0.04 THCA.

According to some embodiments of the invention, the composition (iii) comprises the phytocannabinoids listed in the F7 composition of Table 1.

According to some embodiments of the invention, the composition (iii) comprises percentages of phytocannabinoids as listed in the F7 composition of Table 1±10%.

According to some embodiments of the invention, the composition (iv) comprises at least 4 of the phytocannabinoids.

According to some embodiments of the invention, the composition (iv) comprises all of the phytocannabinoids.

According to some embodiments of the invention, the composition (iv) comprises at least 3% of each of the CBC and/or the CBG.

According to some embodiments of the invention, the composition (iv) comprises at least 1% of each of the CBN and/or the THCV.

According to some embodiments of the invention, the composition (iv) further comprises at least one of cannabidiol (CBD) cannabidivarin (CBDV), cannabidivarinic acid (CBDVA), and Cannabigerolic acid (CBGA).

According to some embodiments of the invention, the composition (iv) further comprises cannabidiol (CBD) cannabidivarin (CBDV), cannabidivarinic acid (CBDVA), and cannabigerolic acid (CBGA).

According to some embodiments of the invention, the composition (iv) comprises less than 0.5% of each of the CBD, CBDV, CBDVA and CBGA.

According to some embodiments of the invention, a concentration ratio of the THC, the CBC, the CBG, the CBN, the THCA, the THCV, the CBD, the CBDV, the CBDVA and/or the CBGA in the composition (iv) is 1 THC:0.04-0.07 CBC:0.03-0.06 CBG, 0.01-0.03 CBN:0.004-0.007 THCA:0.005-0.02 THCV:0.0001-0.002 CBD:0.0001-0.002 CBDV:0.0001-0.002 CBDVA:0.0001-0.002 CBGA.

According to some embodiments of the invention, the composition (iv) comprises the phytocannabinoids listed in the crude extract composition of Table 1.

According to some embodiments of the invention, the composition (iv) comprising percentages of phytocannabinoids as listed in the crude extract composition of Table 1±10%.

According to some embodiments of the invention, the composition is not a cannabis extract.

According to some embodiments of the invention, the composition is a liquid chromatography fraction of a cannabis extract.

According to some embodiments of the invention, the liquid chromatography fraction is obtainable by subjecting the cannabis extract to flash chromatography comprising a Flash chromatography apparatus equipped with a diode array detector, as described in the Examples section.

According to some embodiments of the invention:
the composition (i) (F4) is collected between about 6-8 minutes of the flash chromatography;
the composition (ii) (F5) is collected between about 8-9.5 minutes of the flash chromatography; and/or
the composition (iii) (F7) is collected between about 12-14 minutes of the flash chromatography.

According to some embodiments of the invention, the composition is a synthetic composition.

According to some embodiments of the invention, the phytocannabinoids are purified from cannabis.

According to some embodiments of the invention, the cannabis is a cannabis strain Dairy Queen (DQ).

According to some embodiments of the invention, the phytocannabinoids are synthetically synthesized.

According to some embodiments of the invention, the composition is devoid of phytocannabinoids other than the phytocannabinoids.

According to some embodiments of the invention, presence or absence of the phytocannabinoids in the composition is effected by high pressure liquid chromatography (HPLC).

According to some embodiments of the invention, the composition comprises cannabis-derived active ingredients other than phytocannabinoids.

According to some embodiments of the invention:
the composition (i) comprises at least one of the cannabis-derived active ingredients listed in the F4 composition of Table 2;
the composition (ii) comprises at least one of the cannabis-derived active ingredients listed in the F5 or F6 composition of Table 2; and/or
the composition (iii) comprises at least one of the cannabis-derived active ingredients listed in the F7 composition of Table 2.

According to some embodiments of the invention:
the composition (i) comprises the cannabis-derived active ingredients listed in the F4 composition of Table 2;
the composition (ii) comprises the cannabis-derived active ingredients listed in the F5 or F6 composition of Table 2; and/or
the composition (iii) comprises the cannabis-derived active ingredients listed in the F7 composition of Table 2.

According to some embodiments of the invention:
the composition (i) comprises percentages of the cannabis-derived active ingredients as listed in the F4 composition of Table 2±10%;
the composition (ii) comprises percentages of the cannabis-derived active ingredients as listed in the F5 or F6 composition of Table 2±10%; and/or
the composition (iii) comprises percentages of the cannabis-derived active ingredients as listed in the F7 composition of Table 2±10%.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G demonstrate the effect of C. sativa DQ crude extract and fractions F1-11 on viability of A172 GBM cells. FIG. 1A shows cell viability of A172 cells following treatment with C. sativa DQ crude extract or fractions F1-11 at a concentration of 12.5 μg/mL for 48 hours. Cell viability was determined by XTT assay as a function of live cell number. Doxorubicin (Doxo 0.5 μg/mL) served as a positive control; control—vehicle control (0.75% v/v methanol). Error bars indicate ±SE (n=3). Levels with different letters are significantly different from all combinations of pairs according to Tukey-Kramer honest significant difference (HSD; P<0.05). FIG. 1B shows cell viability of A172 cells following treatment with C. sativa DQ crude extract at different concentrations. The IC50 values were calculated from 5P logistic curve fit using GraphPad Prism version 6.1. FIG. 1C is a flash chromatography profile of C. sativa DQ crude extract. As the indicated times, fractions were collected and designated as F1-F11. Approximate range of the THC peak is shown. FIGS. 1D-G show cell viability of A172 cells following treatment with C. sativa DQ fractions F4 (FIG. 1D), F5 (FIG. 1E), F6 (FIG. 1F) or F7 (FIG. 1G) at different concentrations. The IC50 values were calculated from 5P logistic curve fit using GraphPad Prism version 6.1. Error bars indicate ±SE (n=3).

FIGS. 2A-D demonstrate the effect of standard mixes (SM) of pure cannabinoids mimicking fractions F4 and F5 on viability of A172 GBM cells. Shown cell viability following 48 hours treatment with F4-SM (FIG. 2A), F5-SM (FIG. 2B), CBG (FIG. 2C) or THC (FIG. 2D) at different concentrations, as determined by XTT assay as a function of live cell number. The IC50 values were calculated from 5P logistic curve fit by GraphPad Prism version 6.1. Error bars indicate ±SE (n=3).

FIGS. 3A-B demonstrate the effect of F4-SM and S5-SM on apoptosis and cell cycle arrest. FIG. 3A shows percentage of viability, apoptosis, or necrosis in A172 cells following 48 hours treatment with F4-SM (10 μg/mL) or F5-SM (10 μg/mL). FIG. 3B shows percentage of A172 cells in G0/G1, G2/M and S phase following 24 hours treatment with F4-SM (10 μg/mL) or F5-SM (10 μg/mL). 10⁴ cells were analyzed per treatment. Control is vehicle control (1% v/v methanol) and doxorubicin (Doxo, 0.5 μg/mL) served as positive control. The treated cells were harvested and analyzed in FACS following annexin V-FITC and PI staining. Error bars indicate ±SE (n=3). Levels with different letters are significantly different from all combinations of pairs according to the Tukey-Kramer honest significant difference (HSD; P<0.05).

FIGS. 4A-C demonstrate cell viability of A172 cells following 48 hours treatment with F4-SM (FIG. 4A) or F5-SM (FIG. 4B), with or without a CB1 or CB2 inverse agonist (IA), a TRPA1 blocker (B) or a TRPV1 or TRPV2 antagonist (AN). FIG. 4A shows the effect of IA, B or NA on cell viability. F4-SM was provided at a 12 μg/mL concentration, F5-SM was provided at a 10 μg/mL concentration, the receptors IA, B or AN were provided at a 10 μM concentration. Cell viability was determined by XTT assay as a function of live cell number. Doxorubicin (Doxo, 0.5 μg/mL) served as a positive control. Control—vehicle control (1.1% v/v methanol+1% DMSO). Error bars indicate ±SE (n=3). Levels with different letters are significantly different from all combinations of pairs according to the Tukey-Kramer honest significant difference (HSD; P<0.05).

FIGS. 5A-F demonstrate quantitative PCR determination of the RNA steady state level of ATF4 (FIGS. 5A-B) TRIB3 (FIGS. 5C-D) and CHOP (DDIT3-3) (FIGS. 5E-F) genes in A172 cell line following treatment with F4-SM or F5-SM (10 μg/mL) relative to control [vehicle control (1.2% v/v methanol)]. Gene transcript values were determined by quantitative PCR. Mean values ±SE are shown (n=3).

FIGS. 6A-D demonstrate the effect of 24 hours (FIG. 6A-B) or 48 hours (FIGS. 6C-D) treatment with F4-SM or F5-SM (10 μg/mL) on viability of GSCs. Live/dead cell count was determined by trypan blue exclusion assay (FIGS. 6A and 6C); LDH assay was used to determine cell death relative to vehicle control (FIGS. 6B and 6D). Error bars indicate ±SE (n=3).

FIGS. 7A-B demonstrate the effect of F4, F5, F4-SM or F5-SM on A172 cell migration. Cells were treated with F4 (20 μg/mL) or F4-SM (11.5 μg/mL) (FIG. 7A); F5 (20 μg/mL) or F5-SM (11.5 μg/mL) (FIG. 7B). Control—vehicle control (1.15% v/v methanol). Doxorubicin (Doxo, 0.5 μg/mL) served as a positive control. Percent scratch area is presented as mean; error bars indicate ±SE (n=3). Levels with different letters are significantly different from all combinations of pairs according to the Tukey-Kramer honest significant difference (HSD; P<0.05).

FIGS. 8A-B demonstrate the effect of F4 and F5 on the cytoskeleton. FIG. 8A shows representative confocal images of A172 cells following 24 hours treatment with F4 (12 μg/mL) or F5 (10 μg/mL). F-actin (EasyProbes™ ActinRed 555 Stain, red stain), and nuclei (Hoechst, blue stain) were stained. Control—vehicle control (1.15% v/v methanol). Doxorubicin (Doxo, 0.5 μg/mL) served as a positive control. Scale bars=20 μm; n=3. Yellow arrows point to disintegration of F-actin filaments visualized as characteristic spots; green arrows show F-actin filopodia protruding from cells. FIG. 8B is a graph quantifying the number of F-actin filaments that crossed a line drawn across the soma in each of the treatments. Percent number of filaments is presented as mean; error bars indicate ±SE (n=5). Levels with different letters are significantly different from all combinations of pairs according to the Tukey-Kramer honest significant difference (HSD; P<0.05).

FIGS. 9A-C demonstrate the effect of the F4 and F5 treatments on A172 cell invasion, determined by the vertical movement of A172 cells across the 8-μm pore size membrane, following 24 hours treatment with F4 (12 μg/mL) or F5 (10 μg/mL). Error bars indicate ±SE (n=3). Levels with different letters are significantly different from all combinations of pairs according to the Tukey-Kramer honest significant difference (HSD; P<0.05). FIG. 9A shows percentage of cell viability; FIG. 9B shows percentage of invading cell in comparison to control; FIG. 9C shows representative images of crystal violet staining of cells that invaded the membrane at 24 hours. Scale bars=500 μm. Doxorubicin (Doxo, 0.5 μg/mL) served as a positive control. Control—vehicle control (0.6% v/v methanol).

FIGS. 10A-D demonstrate the effect of F4 and F5 on colony formation of A172 and U87 cells. Shown is the percentage of colonies (out of the average number of colonies in the control at the highest cell concentration [6×10 4]) following treatment of A172 with F4 (20 μg/mL) or F5 (16.5 μg/mL) (FIG. 10A) or U87 cells with F4 (12 μg/mL) or F5 (10 μg/mL) (FIG. 10B). Cells were sorted to live cells and these were seeded under conditions that promote neurosphere formation. Error bars indicate ±SE (n=3). Levels with different letters are significantly different from all combinations of pairs according to the Tukey-Kramer honest significant difference (HSD; P<0.05). FIGS. 10C-D show representative images used to count colonies in 6×10 4 cell cultures. Scale bars=500 μm. Control is vehicle control (0.5% v/v methanol).

FIGS. 11A-B show the effect of F4, F4-SM, F5 and F5-SM on colony formation in 3D structures. FIG. 11A shows representative confocal images of U87 cells following 8 days treatment with F4 (20 μg/mL), F5 (16.5 μg/mL), F4-SM (12.5 μg/mL) or F5-SM (10 μg/mL). Doxorubicin (Doxo, 2.0 μg/mL) served as a positive control. Control—vehicle control (2.0% v/v methanol). Overlay of brightfield and Hoechst staining for nuclei (blue color) is shown in 5 concurrent optical sections; Scale bars=200 μm; n=3. Arrows point to some of the colonies that formed. FIG. 11B shows the percentage of number of colonies in each of the treatments (out of the average number of colonies in the control), presented as mean; error bars indicate ±SE (n=3). Levels with different letters are significantly different from all combinations of pairs according to the Tukey-Kramer honest significant difference (HSD; P<0.05).

FIG. 12 demonstrate no effect of temozolomide (TMZ) on viability of A172 GBM cells. Cells were treated with the indicated concentrations of TMZ for 48 hours. Cell viability was determined by XTT assay as a function of live cell number. Doxorubicin (Doxo 0.5 μg/mL) served as positive controls; control—solvent (vehicle) control (0.5% v/v DMSO). Error bars indicate ±SE (n=3). Levels with different letters are significantly different from all combinations of pairs by Tukey-Kramer honest significant difference (HSD; P<0.05).

FIGS. 13A-B demonstrate the effect of F4 and F5 C. sativa DQ fractions and synthetic mixes on viability of U87 GBM cells. FIG. 13A shows cell viability of U87 cells following 48 hours treatment with F4, F5, F4-SM or F5-SM at the indicated concentrations. FIG. 13B shows cell viability of U87 cells following 48 hours treatment with doxorubicin (Doxo) at indicated concentrations. Cell viability was determined by XTT assay as a function of live cell number. Control—solvent (vehicle) control (1.5% v/v methanol). Error bars indicate ±SE (n=3). Levels with different letters are significantly different from all combinations of pairs according to the Tukey-Kramer honest significant difference (HSD; P<0.05).

FIGS. 14A-B demonstrate the effect of F4-SM and S5-SM on apoptosis and cell cycle arrest. FIG. 14A shows Annexin V-FITC and PI staining for determining the proportion of viable (Q4), apoptotic (Q2 and Q3 for late and early apoptosis, respectively) or necrotic cells (Q1). FIG. 14B shows an example of FACS output following PI staining to determine the stages of cell cycle arrest. Treatments included F4-SM (10 μg/mL) or F5-SM (10 μg/mL) on the A172 cell line for 24 hours for cell cycle and 48 hours for apoptosis. Doxorubicin (Doxo, 0.5 μg/mL) served as a positive control. Methanol (control) treatment served as a solvent (vehicle) control.

FIG. 15 demonstrates the effect of F4, F4-SM, F5 and F5-SM on cell migration. Shown are representative pictures taken for estimating the effectiveness of treatment with F4 (20 μg/mL), F4-SM (11.5 μg/mL), F5 (20 μg/mL), F5-SM (11.5 μg/mL) or doxorubicin (Doxo, 0.5 μg/mL) on recovered area of confluent monolayers of the A172 cell line at 0, 14, 20 and 36 hours post treatment. Methanol (control) treatment served as a solvent (vehicle) control.

FIG. 16 is a schematic representation of the processes conducted in the Examples section which follows.

FIGS. 17A-B show cell viability of HTB75 cells (FIG. 17A) following treatment with crude extract of cannabis strains DQ, Paris, GB-11, GB-14 and GB-18. Cell viability was determined by XTT assay as a function of live cell number. NT is the vehicle treated control (1% v/v methanol). Error bars indicate ±SE (n=3). Levels with different letters are significantly different from all combinations of pairs by Tukey-Kramer honest significant difference (HSD; P≤0.05). (FIG. 17B) following treatment with C. sativa DQ crude extract at different concentrations for IC50 value calculation from 5P logistic curve fit using GraphPad Prism version 6.1. Error bars indicate ±SE (n=3).

FIGS. 18A-C show cell viability of HTB75 cells following treatment with DQ extract fractions F1-F8, and crude extract. FIG. 18A—Cell viability was determined by XTT assay as a function of live cell number. NT is the vehicle treated control (1% v/v methanol). Error bars indicate ±SE (n=3). Levels with different letters are significantly different from all combinations of pairs by Tukey-Kramer honest significant difference (HSD; P<0.05). (FIGS. 18B-C) Cell viability of HTB75 cells following treatment with C. sativa DQ fractions F5 and F7 at different concentrations for IC50 values calculation from 5P logistic curve fit using GraphPad Prism version 6.1. Error bars indicate ±SE (n=3).

FIGS. 19A-B show cell viability of HTB75 cells following treatment with F5-SM (FIG. 19A) and F7-SM (FIG. 19B) at different concentrations for IC50 values calculation from 5P logistic curve fit using GraphPad Prism version 6.1. Error bars indicate ±SE (n=3).

FIGS. 20A-B show cell viability of HTB75 cells following treatment with THC or with combinations of THC, CBG, CBC and CBN as in F7 or F5, at 13 (FIG. 20A) and 15 (FIG. 20B) μg/mL (treatments are listed in Table 6). Cell viability was determined by XTT assay as a function of live cell number. All calculation are in relation to NT (the vehicle treated control of 1.5% v/v methanol) that is considered 100% cell viability but is not shown. Error bars indicate ±SE (n=3). Levels with different letters are significantly different from all combinations of pairs by Tukey-Kramer honest significant difference (HSD; P<0.05).

FIG. 21A shows the percentage of viability, apoptosis, or necrosis in HTB75 cells following treatment with niraparib (5.1 μg/mL), F5 (19.1 μg/mL), F7 (19.4 μg/mL), F5-SM (17.4 μg/mL) or F7-SM (16.3 μg/mL), for 48 h.

FIG. 21B shows the percentage of HTB75 cells in G1, S or G2/M phase following treatment as in 21(A) except 4 μg/mL niraparib, for 24 h. 10⁴ cells were analyzed per treatment. Control is vehicle control (1.5% v/v methanol). The treated cells were harvested and analyzed in FACS following annexin V-FITC and PI staining. Error bars indicate ±SE (n=3). Levels with different letters are significantly different from all combinations of pairs according to the Tukey-Kramer honest significant difference (HSD; P<0.05).

FIGS. 22A-D shows cell viability of HTB75 cells following treatment with F5 (a), F7 (b), F5-SM (c) or F7-SM (d), with or without CB1 and CB2 inverse agonists (IA), a TRPA1 blocker (B) and TRPV1 or TRPV2 antagonists (AN) for 48 h. Cells were treated with F5 (19.1 μg/mL), F7 (19.4 μg/mL), F5-SM (14.7 μg/mL) or F7-SM (13.6 μg/mL), with or without the receptors IA, B or AN (10 μM).

FIG. 22E shows the effect of IA, B or NA on cell viability. Cell viability was determined by XTT assay as a function of live cell number. Niraparib (6 μg/mL) served as a positive control. Control is vehicle control (1.5% v/v methanol+1% DMSO). Error bars indicate ±SE (n=3). Levels with different letters are significantly different from all combinations of pairs according to the Tukey-Kramer honest significant difference (HSD; P<0.05).

FIGS. 23A-C are graphs showing synergistic interactions between fractions of some embodiments of the invention with (FIG. 23A) cisplatin, (FIG. 23B) gemcitabine and (FIG. 23C) niraparib on cell viability of HTB75 cells following combined treatments. Synergy of cytotoxic activity calculated based on Bliss-independence drug interaction model. Synergy is apparent when the experimental (observed) value of cell survival inhibition is higher than the calculated (expected) value. Values of delta of observed minus expected values, calculated based on Bliss model are shown in the Y axis.

FIG. 24 is a graph showing quantitative PCR-based determination of the RNA steady state level in HTB75 cell line of MAPK4 gene, after treatment with niraparib, F5, F7 or combination of niraparib+F5 or niraparib+F7, for 2 h relative to control. Gene transcript values were determined by quantitative PCR as a ratio between the target gene versus a reference gene (Hypoxanthine Phosphoribosyltransferasel; HPRT1; geneID 3251). Values were calculated relative to the average expression of target genes in treated versus control using the 2ΔΔCt method. Control (methanol) treatment served as solvent (vehicle) control; Error bars indicate ±s.e.m. (n=3). Levels with different letters are significantly different from all combinations of pairs by Tukey-Kramer honest significant difference (HSD; P<0.05).

FIGS. 25A-D show the effect of niraparib+avastin or niraparib+avastin+F7 on xenograft mice model. (a) Tumor volume of mice treated with niraparib 25 mg/kg+avastin 5 mg/k; niraparib 25 mg/kg+avastin 5 mg/kg+F7 50 mg/kg; or vehicle control (10% v/v methanol in saline). Levels with different letters with the same font and style are significantly different from all combinations of pairs by Tukey-Kramer honest significant difference (HSD; P<0.05). *P<0.05 in niraparib+avastin+F7 vs. niraparib+avastin group based on Student's t-test. (b) Representative pictures of mice and tumors. (c) Body weight of the treated mice. Levels with different letters with the same font and style are significantly different from all combinations of pairs by Tukey-Kramer honest significant difference (HSD; P<0.05). *P<0.05 in niraparib+avastin+F7 vs. niraparib+avastin group based on Student's t-test. (d) Representative pictures of histological H&E staining of tumors. Red arrows indicate neoplastic cells, yellow arrow denote necrotic tissue. Error bars indicate ±S.E. (n=3).

### DESCRIPTION OF SPECIFIC EMBODIMENTS OF THE INVENTION

The present invention, in some embodiments thereof, relates to compositions and methods for the treatment of cancer.

Marijuana (Cannabis sativa) contains more than 500 constituents, among them phytocannabinoids, terpenes and flavonoids. Cannabinoids were previously shown to prevent proliferation, metastasis, angiogenesis and induce apoptosis in a variety of cancer cell types.

Whilst searching for Cannabis sativa extracts which are particularly effective in combatting cancer, the present inventors have screened a plurality of varieties and found one such variety, Dairy Queen (DQ) (IMC, Israel), high CBD strain of C. sativa from IMC, an ethanol extract of which was found to be endowed with an unprecedented anti-cancer activity. The present inventors fractionated the extract and identified HPLC-defined fractions that have various anti-cancer activities including, cell-cycle arrest, cell death induction, inhibition of cell migration, inhibition of colony formation and more. The effect was exemplified on a plurality of cancer samples. In addition, the present inventors were able to identify active ingredients in these fractions by formulating synthetic compositions which comprise the defined ingredients. Synergy was exemplified between different fractions or synthetic compositions and in the presence of chemotherapy and antibody therapy (e.g., anti-VERGF-A).

Thus, according to an aspect of the invention there is provided a method of treating cancer in a subject in need thereof, the method comprising administering to the subject a therapeutically effective amount of a composition selected from the group consisting of:
(i) a composition comprising phytocannabinoids, wherein the phytocannabinoids comprise at least 70% cannabigerol (CBG) and at least one phytocannabinoid selected from the group consisting of cannabinol (CBN) and tetrahydrocannabivarin (THCV);
(ii) a composition comprising phytocannabinoids, wherein the phytocannabinoids comprise at least 85% tetrahydrocannabinol (THC) and at least 1.5% cannabigerol (CBG);
(iii) a composition comprising phytocannabinoids, wherein the phytocannabinoids comprise at least 50% tetrahydrocannabinol (THC), at least 10% cannabichromene (CBC) and at least one phytocannabinoid selected from the group consisting of cannabidivarin (CBDV), cannabidivarinic acid (CBDVA), cannabigerol (CBG), cannabinol (CBN) and tetrahydrocannabinolic acid (THCA); and
(iv) a composition comprising phytocannabinoids, wherein the phytocannabinoids comprise at least 80% tetrahydrocannabinol (THC) and at least three phytocannabinoids selected from the group consisting of cannabichromene (CBC), cannabigerol (CBG), cannabinol (CBN), tetrahydrocannabinolic acid (THCA) and tetrahydrocannabivarin (THCV), thereby treating the cancer in the subject.

Alternatively or additionally, there is provided a composition selected form the group consisting of:
(i) a composition comprising phytocannabinoids, wherein the phytocannabinoids comprise at least 70% cannabigerol (CBG) and at least one phytocannabinoid selected from the group consisting of cannabinol (CBN) and tetrahydrocannabivarin (THCV);
(ii) a composition comprising phytocannabinoids, wherein the phytocannabinoids comprise at least 85% tetrahydrocannabinol (THC) and at least 1.5% cannabigerol (CBG);
(iii) a composition comprising phytocannabinoids, wherein the phytocannabinoids comprise at least 50% tetrahydrocannabinol (THC), at least 10% cannabichromene (CBC) and at least one phytocannabinoid selected from the group consisting of cannabidivarin (CBDV), cannabidivarinic acid (CBDVA), cannabigerol (CBG), cannabinol (CBN) and tetrahydrocannabinolic acid (THCA); and
(iv) a composition comprising phytocannabinoids, wherein the phytocannabinoids comprise at least 80% tetrahydrocannabinol (THC) and at least three phytocannabinoids selected from the group consisting of cannabichromene (CBC), cannabigerol (CBG), cannabinol (CBN), tetrahydrocannabinolic acid (THCA) and tetrahydrocannabivarin (THCV),
for use in treating cancer in a subject in need thereof.

Alternatively or additionally, there is provided a method of reducing viability, inducing cell cycle arrest and/or reducing proliferation and/or migration of a cancerous cell, the method comprising contacting the cancerous cell with a composition selected from the group consisting of:
(i) a composition comprising phytocannabinoids, wherein the phytocannabinoids comprise at least 70% cannabigerol (CBG) and at least one phytocannabinoid selected from the group consisting of cannabinol (CBN) and tetrahydrocannabivarin (THCV);
(ii) a composition comprising phytocannabinoids, wherein the phytocannabinoids comprise at least 85% tetrahydrocannabinol (THC) and at least 1.5% cannabigerol (CBG);
(iii) a composition comprising phytocannabinoids, wherein the phytocannabinoids comprise at least 50% tetrahydrocannabinol (THC), at least 10% cannabichromene (CBC) and at least one phytocannabinoid selected from the group consisting of cannabidivarin (CBDV), cannabidivarinic acid (CBDVA), cannabigerol (CBG), cannabinol (CBN) and tetrahydrocannabinolic acid (THCA); and
(iv) a composition comprising phytocannabinoids, wherein the phytocannabinoids comprise at least 80% tetrahydrocannabinol (THC) and at least three phytocannabinoids selected from the group consisting of cannabichromene (CBC), cannabigerol (CBG), cannabinol (CBN), tetrahydrocannabinolic acid (THCA) and tetrahydrocannabivarin (THCV).

Alternatively or additionally, there is provided a composition selected from the group consisting of:
(i) a composition comprising the phytocannabinoids listed in the F4 composition of Table 1 in percentages as listed in the F4 composition of Table 1±10%;
(ii) a composition comprising the phytocannabinoids listed in the F5 composition of Table 1 in percentages as listed in the F5 composition of Table 1±10% or a composition comprising the phytocannabinoids listed in the F6 composition of Table 1 in percentages as listed in the F6 composition of Table 1±10%;
(iii) a composition comprising the phytocannabinoids listed in the F7 composition of Table 1 in percentages as listed in the F7 composition of Table 1±10%;
(iv) a composition comprising the phytocannabinoids listed in the crude extract composition of Table 1 in percentages as listed in the crude extract composition of Table 1±10 15%;
(v) a composition comprising the phytocannabinoids listed in the sCBD crude extract composition of Table 7 in percentages as listed in the crude extract composition of Table 7±10%; and
(vi) a composition comprising the phytocannabinoids listed in the PARIS crude extract composition of Table 7 in percentages as listed in the crude extract composition of Table 7±10%.

Alternatively or additionally, there is provided a method of treating cancer in a subject in need thereof, the method comprising administering to the subject a therapeutically effective amount of the composition as described hereinabove and further detailed hereinbelow, thereby treating the cancer in the subject.

Alternatively or additionally, there is provided a composition as described hereinabove and further detailed hereinbelow, for use in treating cancer in a subject in need thereof.

Alternatively or additionally, there is provided a method of reducing viability, inducing cell cycle arrest and/or reducing proliferation and/or migration of a cancerous cell, the method comprising contacting the cancerous cell with the composition as described hereinabove and further detailed hereinbelow.

When “±10%” is indicated it refers to a range for a specific component within an extract or a fraction, e.g., CBG in F4, a plurality of components in an extract or a fraction, e.g., at least 2, or all components within an extract or a fraction. The range can be up to ±10%, e.g., up to ±9%, ±8%, ±7%, ±6%, ±5%, ±4%, ±3%, ±2%, ±1% or less, as long as it is not the same value indicated in the Table e.g., Table 1 or Table 7.

According to a specific embodiment, the term “phytocannabinoid” refers to a meroterpenoid with a resorcinyl core typically decorated with a para-positioned isoprenyl, alkyl, or aralkyl side chain originated from a cannabis plant, acidic or decarboxylated acid forms thereof. The term also reads on synthetic analogs or derivatives of the plant originated substance.

Alternatively or additionally, the term “phytocannabinoid” refers to a cannabinoid selected from the list provided in Table 8 hereinbelow originated from a cannabis plant, acidic or decarboxylated acid forms thereof. The term also reads on synthetic compositions and analogs or derivatives of the plant originated substance.

TABLE 8
List of neutral phytocannabinoids, decarboxylated acid forms
(modified from Berman P, Futoran K, Lewitus G M, Mukha D,
Benami M, Shlomi T, Meiri D. A new ESI-LC/MS approach for
comprehensive metabolic profiling of phytocannabinoids in
Cannabis. Scientific reports. 2018 Sep. 24; 8(1): 1-5.)

Cannabiorcol-C1 (CBNO)
CBND-C1 (CBNDO)
(−)-Δ 9-trans-Tetrahydrocannabiorcol-C1 (Δ9 -THCO)
Cannabidiorcol-C1 (CBDO)
Cannabiorchromene-C1 (CBCO)
(−)-Δ 8-trans-(6aR,10aR)-Tetrahydrocannabiorcol-C1 (Δ8-THCO)
Cannabiorcyclol C1 (CBLO)
CBG-C1 (CBGO)
Cannabinol (CBN)
CBND-C2
Delta-9-tetrahydrocannabinol Δ9-THC (THC)
Cannabidiol (CBD)
Δ8-THC-C2
CBL-C2
Bisnor-cannabielsoin-C1 (CBEO)
Cannabigerol (CBG)
Cannabivarin-C3 (CBNV)
Cannabinodivarin-C3 (CBNDV)
Δ9-trans-Tetrahydrocannabivarin Δ9-THCV (THCV)
(−)-Cannabidivarin-C3 (CBDV)
(±)-Cannabichromevarin-C3 (CBCV)
(−)-Δ8-trans-THC-C3 (Δ8-THCV)
Δ7-tetrahydrocannabivarin-C3 (Δ7-THCV)
(±)-(1aS,3aR,8bR,8cR)-Cannabicyclovarin-C3 (CBLV)
2-Methyl-2-(4-methyl-2-pentenyl)-7-propyl-2H-1-benzopyran-5-ol
CBE-C2
Cannabigerovarin-C3 (CBGV)
Cannabitriol-C1 (CBTO)
Cannabinol-C4 (CBN-C4)
CBND-C4
(−)-Δ9-trans-Tetrahydrocannabinol-C4
(Δ9-THC-C4)
Cannabidiol-C4 (CBD-C4)
Cannabichromene (CBC)
(−)-trans-Δ8-THC-C4
CBL-C4
Cannabielsoin-C3 (CBEV)
CBG-C4
CBT-C2
Cannabichromanone-C3
Cannabiglendol-C3 (OH-iso-HHCV-C3)
Cannabioxepane-C5 (CBX)
Dehydrocannabifuran-C5 (DCBF)
Cannabinol-C5 (CBN)
Cannabinodiol-C5 (CBND)
Cannabifuran-C5 (CBF)
(−)-Δ9-trans-Tetrahydrocannabinol-C5
(Δ9-THC)
(−)-Δ8-trans-(6aR,10aR)-
Tetrahydrocannabinol-C5 (Δ8-THC)
(±)-Cannabichromene-C5 (CBC)
(−)-Cannabidiol-C5 (CBD)
(±)-(1aS,3aR,8bR,8cR)-Cannabicyclol-
C5 (CBL)
Cannabicitran-C5 (CBR)
(−)-Δ9-(6aS,10aR-cis)-
Tetrahydrocannabinol-C5 ((−)-cis-Δ9-
THC)
(−)-Δ7-trans-(1R,3R,6R)-
Isotetrahydrocannabinol-C5
(trans-iso-Δ7-THC)
CBE-C4
Cannabigerol-C5 (CBG)
Cannabitriol-C3 (CBTV)
Cannabinol methyl ether-C5 (CBNM)
CBNDM-C5
8-OH-CBN-C5 (OH-CBN)
OH-CBND-C5 (OH-CBND)
10-Oxo-Δ6a(10a)-Tetrahydrocannabinol-
C5 (OTHC)
Cannabichromanone D-C5
Cannabicoumaronone-C5 (CBCON-C5)
Cannabidiol monomethyl ether-C5
(CBDM)
Δ9-THCM-C5
(±)-3″-hydroxy-Δ4″-cannabichromene-C5
(5aS,6S,9R,9aR)-Cannabielsoin-C5
(CBE)
2-geranyl-5-hydroxy-3-n-pentyl-1,4-
benzoquinone-C5
8α-Hydroxy-Δ9-Tetrahydrocannabinol-
C5 (8α-OH-Δ9-THC)
8β-Hydroxy-Δ9-Tetrahydrocannabinol-
C5 (8β-OH-Δ9-THC)
10α-Hydroxy-Δ8-Tetrahydrocannabinol-
C5 (10α-OH-Δ8-THC)
10β-Hydroxy-Δ8-Tetrahydrocannabinol-
C5 (10β-OH-Δ8-THC)
10α-hydroxy-49,11-hexahydrocannabinol-
C5
9β,10β-Epoxyhexahydrocannabinol-C5
OH-CBD-C5 (OH-CBD)
Cannabigerol monomethyl ether-C5
(CBGM)
Cannabichromanone-C5
CBT-C4
(±)-6,7-cis-epoxycannabigerol-C5
(±)-6,7-trans-epoxycannabigerol-C5
(−)-7-hydroxycannabichromane-C5
Cannabimovone-C5
(−)-trans-Cannabitriol-C5 ((−)-trans-
CBT)
(±)-trans-Cannabitriol-C5 ((+)-trans-
CBT)
(±)-cis-Cannabitriol-C5 ((±)-cis-CBT)
(−)-trans-10-Ethoxy-9-hydroxy-Δ6a(10a)-
tetrahydrocannabivarin-C3 [(−)-trans-
CBT-OEt]
(−)-(6aR,9S,10S,10aR)-9,10-
Dihydroxyhexahydrocannabinol-C5 [(−)-
Cannabiripsol] (CBR)
Cannabichromanone C-C5
(−)-6a,7,10a-Trihydroxy-Δ9-
tetrahydrocannabinol-C5 [(−)-
Cannabitetrol] (CBTT)
Cannabichromanone B-C5
8,9-Dihydroxy-Δ6a(10a)-
tetrahydrocannabinol-C5 (8,9-Di-OHCBT)
(±)-4-acetoxycannabichromene-C5
2-acetoxy-6-geranyl-3-n-pentyl-1,4-
benzoquinone-C5
11-Acetoxy-Δ9-Tetrahydrocannabinol-
C5 (11-OAc-Δ9-THC)
5-acetyl-4-hydroxycannabigerol-C5
4-acetoxy-2-geranyl-5-hydroxy-3-npentylphenol-
C5
(−)-trans-10-Ethoxy-9-hydroxy-Δ6a(10a)-
tetrahydrocannabinol-C5 ((−)-trans-CBTOet)
4-acetoxy-2-geranyl-5-hydroxy-3-npropylphenol-
C5
sesquicannabigerol-C5 (SesquiCBG)
carmagerol-C5
4-terpenyl cannabinolate-C5
β-fenchyl-Δ9-tetrahydrocannabinolate-
C5
α-fenchyl-Δ9-tetrahydrocannabinolate-
C5
epi-bornyl-Δ9-tetrahydrocannabinolate-
C5
bornyl-Δ9-tetrahydrocannabinolate-C5
α-terpenyl-Δ9-tetrahydrocannabinolate-
C5
4-terpenyl-Δ9-tetrahydrocannabinolate-
C5

As used herein, a “percent (%) of a phytocannabinoid” in the compositions disclosed herein refers to the concentration as presented in percentage (w/v) of the recited phytocannabinoid out of the total phytocannabinoids (and only the phytocannabinoids) in the composition, as can be determined by the peak area according to a HPLC profile of the composition.

According to specific embodiments, the concentration ratio is determined by g/l:g/l or μg/ml:μg/ml.

As used herein “a fraction” refers to a portion of the extract that contains only certain chemical ingredients of the extract but not all.

Herein, the term “synthetic composition” refers to a chemically defined composition which can include active ingredients which are chemically synthesized and/or purified to a level of purity of at least 99%.

As used herein “a chemically defined composition” refers to a composition in which all the constituents are known by structure and optionally concentration.

Cannabichromene (CBC) (CAS NO. 20675-51-8) as used herein encompasses native CBC (i.e. originating from the Cannabis plant), or synthetic analogs or derivatives thereof. According to specific embodiments, any CBC analog may be used in accordance with specific embodiments of the present teachings as long as it comprises the anti-cancer activity described herein (alone, or as part of a fraction or composition discussed herein).

Non-limiting examples of CBC analogs include isocannabichromene, cannabichromene-c0, cannabichromene-c1, isocannabichromene-c0, CBCan. CBC or CBC-like derivatives found in Rhododendron anthopogonoides.

According to specific embodiments, the CBC comprises native CBC.

Pure or synthetic CBC can be commercially obtained from e.g. Restek catalog no. 34092, Cayman chemicals catalog no. 26252-10/5/50, Santa Cruz Biotechnology catalog no. sc-504602.

Tetrahydrocannabinol (THC) (CAS No. 1972-08-3) as used herein encompasses native THC (i.e. originating from the Cannabis plant), or synthetic analogs or derivatives thereof. According to specific embodiments, any THC analog may be used in accordance with the present teachings as long as it comprises the anti-cancer activity described herein (alone, or as part of the composition discussed herein).

According to specific embodiments, the THC comprises native THC.

Pure or synthetic THC can be commercially obtained from e.g. Restek catalog no. 34067.

The term THC does not include tetrahydrocannabinolic acid (THCA).

Cannabidiol (CBD) (CAS No. 13956-29-1), as used herein, encompasses native CBD (i.e. originating from the Cannabis plant), or synthetic or naturally occurring analogs or derivatives thereof. According to specific embodiments, any CBD analog may be used in accordance with specific embodiments of the present teachings as long as it comprises the anti-cancer activity described herein (alone, or as part of a composition discussed herein).

Exemplary CBD analogs include, but are not limited to, (−)-DMH-CBD-11-oic acid, HU-308 (commercially available e.g. from Tocris Bioscience, 3088), O-1602 (commercially available e.g. from Tocris Bioscience 2797/10), DMH-CBD (commercially available e.g. from Tocris Bioscience, 1481) [as discussed in detail in Burstein S, Bioorg Med Chem. (2015) 23(7): 1377-85], Abn-CBD, HUF-101. CBDV, CBDM, CBND-05, CBND-C3, 6-Hydroxy-CBD-triacetate or CBD-aldehyde-diacetate [as discussed in detail in An Overview on Medicinal Chemistry of Synthetic and Natural Derivatives of Cannabidiol, Frontiers in Pharmacology, June 2017|Volume 8|Article 422].

According to specific embodiments, the CBD is not CBDV.

According to specific embodiments, the CBD comprises native CBD.

Pure or synthetic CBD can be commercially obtained from e.g. Restek catalog no. 34011.

Tetrahydrocannabinolic acid (THCA) (CAS No: 23978-85-0), as used herein, refers to Δ9-tetrahydrocannabinolic acid, the precursor of tetrahydrocannabinol (THC). The term THCA as used herein encompasses native THCA (i.e. originating from the Cannabis plant), or synthetic analogs or derivatives thereof. According to specific embodiments, any THCA analog may be used in accordance with the present teachings as long as it comprises the anti-cancer activity described herein (alone, or as part of the composition discussed herein).

Exemplary THCA analogs include, but are not limited to, 11-OH-delta9-THCA-A and 11-Nor-delta9-THCA-A carboxylic acid [as discussed in detail in Guillermo Moreno-Sanz, Critical Review and Novel Therapeutic Perspectives of D9-Tetrahydrocannabinolic Acid A, Cannabis and Cannabinoid Research Volume 1.1, (2016)].

According to specific embodiments, the THCA comprises native THCA.

Pure or synthetic THCA can be commercially obtained from e.g. Restek catalog no. 34093.

The term THCA does not include tetrahydrocannabinol (THC).

Tetrahydrocannabivarin (THCV) (CAS No. 31262-37-0), as used herein, encompasses native THCV (i.e. originating from the Cannabis plant), or synthetic or naturally occurring analogs or derivatives thereof. According to specific embodiments, any THCV analog may be used in accordance with the present teachings as long as it comprises the anti-cancer activity described herein (alone, or as part of the composition discussed herein).

An exemplary THCV analog include, but is not limited to, Δ8-THCV.

According to specific embodiments, the THCV comprises native THCV.

Pure or synthetic THCV can be commercially obtained from e.g. Restek catalog no. 34100.

Cannabinol (CBN) (CAS NO. 521-35-7), as used herein, encompasses native CBN (i.e. originating from the Cannabis plant), or synthetic analogs or derivatives thereof. According to specific embodiments, any CBN analog may be used in accordance with the present teachings as long as it comprises the anti-cancer activity described herein (alone, or as part of the composition discussed herein).

According to specific embodiments, the CBN comprises native CBN.

Pure or synthetic CBN can be commercially obtained from e.g. Restek catalog no. 34010.

Cannabigerol (CBG) (CAS No. 25654-31-3), as used herein, encompasses native CBG (i.e. originating from the Cannabis plant), or synthetic or naturally occurring analogs or derivatives thereof. According to specific embodiments, any CBG analog may be used in accordance with the present teachings as long as it comprises the anti-cancer activity described herein (alone, or as part of the composition discussed herein).

According to specific embodiments, the CBG comprises native CBG.

Pure or synthetic CBG can be commercially obtained from e.g. Restek catalog no. 34091.

Cannabigerolic acid (CBGA) as used herein, refers to the acidic form of (CBG) and has the formula 3-[(2E)-3,7-Dimethylocta-2,6-dien-1-yl]-2,4-dihydroxy-6-pentylbenzoic acid (CAS No. 255555-57-1). It encompasses native

native CBGA (i.e. originating from the Cannabis plant), or synthetic analogs or derivatives thereof. According to specific embodiments, any CBGA analog may be used in accordance with the present teachings as long as it comprises the anti-cancer activity described herein (alone, or as part of the composition discussed herein).

According to specific embodiments, the CBGA comprises native CBGA.

Pure or synthetic CBGA can be commercially obtained from e.g. Restek catalog no. 34135.

Cannabidiolic acid (CBDA) (CAS No. 1244-58-2), as used herein, encompasses native CBDA (i.e. originating from the Cannabis plant), or synthetic analogs or derivatives thereof. According to specific embodiments, any CBDA analog may be used in accordance with the present teachings as long as it comprises the anti-cancer activity described herein (alone, or as part of the composition discussed herein).

According to specific embodiments, the CBDA comprises native CBDA.

Pure or synthetic CBDA can be commercially obtained from e.g. Restek catalog no. 34099.

Cannabidivarin (CBDV) (CAS No. 24274-48-4), as used herein, encompasses native CBDV (i.e. originating from the Cannabis plant), or synthetic analogs or derivatives thereof.

According to specific embodiments, any CBDV analog may be used in accordance with the present teachings as long as it comprises the anti-cancer activity described herein (alone, or as part of the composition discussed herein).

According to specific embodiments, the CBDV comprises native CBDV.

Cannabidivarinic Acid (CBDVA) (CAS No. 31932-13-5), as used herein, encompasses native CBDVA (i.e. originating from the Cannabis plant), or synthetic analogs or derivatives thereof. According to specific embodiments, any CBDVA analog may be used in accordance with the present teachings as long as it comprises the anti-cancer activity described herein (alone, or as part of the composition discussed herein).

According to specific embodiments, the CBDVA comprises native CBDVA.

Pure or synthetic CBDVA can be commercially obtained from e.g. Restek catalog no 34094.

The compositions disclosed herein comprise cannabinoids at percentages as described herein.

As used herein, a “percent (%) of a cannabinoid” in the fractions and compositions disclosed herein refers to the % calculated from concentration (w/v) of the recited cannabinoid out of the total cannabinoids, active ingredients or compounds in the fraction or composition, as can be determined by the peak area according to a HPLC profile of the fraction or composition.

According to specific embodiments, the % of a cannabinoid is out of the total cannabinoids i.e., phytocannabinoids (and only the cannabinoids i.e., phytocannabinoids) in the fraction or composition.

Methods of determining presence or absence of a compounds in the composition, as well as the concentration of a compound in the composition are well known in the art, such as, but not limited to ultraviolet-visible spectroscopy (“UV-Vis”), infrared spectroscopy (“IR”), and the like; mass-spectrometry (“MS”) methods such as, but not limited to, time-of-flight MS; quadrupole MS; electrospray MS, Fourier-transform MS, Matrix-Assisted Laser Desorption/Ionization (“MALDI”), and the like; chromatographic methods such as, but not limited to, gas-chromatography (“GC”), liquid chromatograph (“LC”), high-performance liquid chromatography (“HPLC”), and the like; and combinations thereof (e.g., GC/MS, LC/MS, HPLC/UV-Vis, and the like), and other analytical methods known to persons of ordinary skill in the art.

According to specific embodiments, determining presence or absence of a compound in the composition and/or the concentration of a compound in the composition is effected by analytical high pressure liquid chromatography (HPLC).

Thus, as mentioned, the composition comprises phytocannabinoids, wherein the phytocannabinoids comprise at least 70% cannabigerol (CBG) and at least one phytocannabinoid selected from the group consisting of cannabinol (CBN) and tetrahydrocannabivarin (THCV);

It will be appreciated that when the percent is indicated it refers to the specific phytocannabinoid following the numerical value. In this case it means that the composition comprises at least 70% CBG.

According to a specific embodiment, the at least 70% CBG comprises at least, 71%, 72%, 73%, 74%, 75%, 76%, 77%, 78%, 79%, 80%, 81%, 82%, 83%, 84%, 85%, 86%, 87%, 88%, 89% or at least 90% CBG.

According to a specific embodiment, the composition (i) comprises at least 75% of the CBG.

According to a specific embodiment, the CBG of the phytocannabinoids comprises 75% to 85%, 70% to 90%, 75% to 95%, 80% to 95% CBG.

CBN and/or THCV can be present in a percentage of up to 30% (e.g., CBN+THCV 10-30%, 10-20%, 5-10%, 5-25%) individually or together.

According to a specific embodiment, the composition comprises CBG+CBN.

According to a specific embodiment, the CBN is at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27% 28% 29% optionally any of which up to 30%.

According to a specific embodiment, the CBN is 2-30%, 2-29%, 5-30%, 5-29%, 7-30%, 7-25%, 5-25%, 10-25%, 10-20%, 10-15%.

According to a specific embodiment, the THCV is at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27% 28% 29% optionally any of which up to 30%.

According to a specific embodiment, the THCV is 5-30%, 5-29%, 10-30%, 10-29%, 11-30%, 11-25%, 10-25%, 15-25%, 10-20%, 10-15%.

According to a specific embodiment, the composition comprises CBG+THCV.

According to a specific embodiment, the composition (i) further comprises cannabidiol (CBD).

According to a specific embodiment, the composition (i) comprises at least 70% CBG and CBN

According to a specific embodiment, the composition (i) comprises all of the phytocannabinoids (i.e., CBG, THCV, CBN and CBD).

According to a specific embodiment, the composition (i) comprises at least 2% of the CBN and/or at least 5% of the THCV.

According to a specific embodiment, the composition (i) comprises at least 4% of the CBD, e.g., at least 4%, 6%, 8, 10%, 12%, 14% 15%, e.g., each of which not exceeding 23%.

According to a specific embodiment, the composition (i) comprises no more than 10% of the CBD.

According to a specific embodiment, the composition (i) is devoid of tetrahydrocannabinol (THC).

According to a specific embodiment, the composition (i) is devoid of tetrahydrocannabinolic acid (THCA), cannabigerolic acid (CBGA), cannabidivarinic acid (CBDVA), cannabidivarin (CBDV) and/or cannabichromene (CBC).

According to a specific embodiment, the concentration ratio of the CBG, the CBN, the THCV and/or the CBD in the composition (i) is 1 CBG:0.01-0.05 CBN:0.05-0.3 THCV:0.06-0.09 CBD.

According to a specific embodiment, the concentration ratio of the CBG, the CBN, the THCV and/or the CBD in the composition (i) is 1 CBG:0.02-0.05 CBN:0.1-0.3 THCV:0.07-0.09 CBD. According to a specific embodiment, the composition (i) comprises the phytocannabinoids listed in the F4 composition of Table 1.

According to a specific embodiment, the concentration ratio of the CBG, the CBN, the THCV and/or the CBD in the composition (i) is 1 CBG:0.02-0.04 CBN:0.1-0.3 THCV:0.07-0.09 CBD. According to a specific embodiment, the composition (i) comprises the phytocannabinoids listed in the F4 composition of Table 1.

According to a specific embodiment, the composition (i) comprises percentages of phytocannabinoids as listed in the F4 composition of Table 1±10%.

Alternatively or additionally and as mentioned, the composition is (ii) a composition comprising phytocannabinoids, wherein the phytocannabinoids comprise at least 85% tetrahydrocannabinol (THC) and at least 1.5% cannabigerol (CBG);

According to a specific embodiment, the at least 85% THC comprises at least, 86%, 87%, 88%, 89%, 90%, 91%, 92%, 93%, 94%, 95%, 96%, 97%, 98% THC and up to 98.5% THC.

According to a specific embodiment, the composition (ii) comprises at least 95% of the THC.

According to a specific embodiment, the THC of the phytocannabinoids comprises 85% to 98.5%, 85% to 95%, 90% to 98.5%, 95% to 98.5% THC.

According to a specific embodiment, the composition of (ii) comprises CBG at a concentration of 1.5%-15%, 1.5%-12%, 1.5%-10%, 1.5%-8%, 1.5%-7%, 1.5%-6%, 1.5%-5%.

According to a specific embodiment, the composition of (ii) comprises at least 1.5%, 2%, 3%, 4%, 5%, 6%, 7%, 8%, 9%, 10% 11, %, 12%, or more but optionally not exceeding 15% CBG.

According to a specific embodiment, the composition (ii) further comprises cannabinol (CBN).

According to a specific embodiment, the composition of (ii) comprises at least 1.5%, 2%, 3%, 4%, 5%, 6%, 7%, 8%, 9%, or more but optionally not exceeding 10% CBN.

According to a specific embodiment, the composition of (ii) comprises CBN at a concentration of 0.5%-10%, 0.5%-8%, 0.5%-7%, 0.5%-6%, 0.5%-5%, 1-5%, 2-5%, 3-5%.

According to a specific embodiment, the composition (ii) comprises less than 10% of the CBG and/or the CBN.

According to a specific embodiment, the composition (ii) comprises less than 5% of the CBG and/or the CBN.

According to a specific embodiment, the composition (ii) comprises at least 90% of the THC.

According to a specific embodiment, the composition (ii) comprises at least 95% of the THC.

According to a specific embodiment, the composition (ii) is devoid of cannabidiol (CBD), cannabidivarin (CBDV) and/or cannabidivarinic acid (CBDVA).

According to a specific embodiment, the composition (ii) is devoid of cannabichromene (CBC), cannabigerolic acid (CBGA), tetrahydrocannabinolic acid (THCA) and/or THCV.

According to a specific embodiment, a concentration ratio of the THC, the CBG and/or the CBN in the composition (ii) is 1 THC:0.03-0.05 CBG:0.04-0.06 CBN.

According to a specific embodiment, the composition (ii) comprises the phytocannabinoids listed in the F5 composition of Table 1.

According to a specific embodiment, the composition (ii) comprises percentages of phytocannabinoids as listed in the F5 composition of Table 1±10%.

According to a specific embodiment, the concentration ratio of the THC, the CBG and/or the CBN in the composition (ii) is 1 THC:0.01-0.03 CBG:0.007-0.015 CBN.

According to a specific embodiment, the concentration ratio of the THC, the CBG and/or the CBN in the composition (ii) is 1 THC:0.01-0.03 CBG:0.007-0.010 CBN.

According to a specific embodiment, the composition (ii) comprises the phytocannabinoids listed in the F6 composition of Table 1.

According to a specific embodiment, the composition (ii) comprises percentages of phytocannabinoids as listed in the F6 composition of Table 1±10%.

Alternatively or additionally, as mentioned, there is provided (iii) a composition comprising phytocannabinoids, wherein the phytocannabinoids comprise at least 50% tetrahydrocannabinol (THC), at least 10% cannabichromene (CBC) and at least one phytocannabinoid selected from the group consisting of cannabidivarin (CBDV), cannabidivarinic acid (CBDVA), cannabigerol (CBG), cannabinol (CBN) and tetrahydrocannabinolic acid (THCA);

According to a specific embodiment, the composition (iii) comprises at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% 45%, 46%, 47%, 48% 49% and up to 49.95% CBC.

According to a specific embodiment, the composition (iii) comprises at least 20% CBC.

According to a specific embodiment, the composition (iii) comprises no more than 49.95% CBC.

According to a specific embodiment, the composition (iii) comprises at least two of (CBDV), cannabidivarinic acid (CBDVA), cannabigerol (CBG), cannabinol (CBN) and tetrahydrocannabinolic acid (THCA).

According to a specific embodiment, the composition (iii) comprises at least three of (CBDV), cannabidivarinic acid (CBDVA), cannabigerol (CBG), cannabinol (CBN) and tetrahydrocannabinolic acid (THCA).

According to a specific embodiment, the composition (iii) comprises at least four of (CBDV), cannabidivarinic acid (CBDVA), cannabigerol (CBG), cannabinol (CBN) and tetrahydrocannabinolic acid (THCA).

According to a specific embodiment, the composition (iii) comprises all of the phytocannabinoids.

According to a specific embodiment, the composition (iii) comprises less than 10% of each of the CBDV, the CBDVA, the CBG the CBN and/or the THCA.

According to a specific embodiment, the composition (iii) comprises less than 9%, 8%, 7%, 6%, 5%, 4%, 3%, 2% of each of the CBDV, the CBDVA, the CBG the CBN and/or the THCA. According to a specific embodiment a minimal amount of 0.05% of at least one of the CBDV, the CBDVA, the CBG the CBN and/or the THCA is provided.

According to a specific embodiment, the composition (iii) comprises less than 5% of each of the CBDV, the CBDVA, the CBG the cannabinol (CBN) and/or the THCA.

According to a specific embodiment, the at least 50% THC comprises at least, 51%, 52%, 53%, 54%, 55%, 56%, 57%, 58%, 59% 60%, 61%, 62%, 63%, 64%, 65%, 66%, 67%, 68%, 69%, 70%, 71%, 72% m 73%, 74%, 75%, 76%, 77%, 78%, 79%, 80%, 81%, 82%, 83%, 84%, 85%, 86%, 87%, 88% and up to 89.95% THC.

According to a specific embodiment, the composition (iii) comprises at least 60% of the THC.

According to a specific embodiment, the composition or the article of manufacture of any one of claims 1-52, wherein the composition (iii) is devoid of cannabidiol (CBD), cannabigerolic acid (CBGA) and/or tetrahydrocannabivarin (THCV).

According to a specific embodiment, a concentration ratio of the THC, the CBC, the CBDV, the CBDVA, the CBG, the CBN and/or the THCA in the composition (iii) is 1 THC:0.2-0.5 CBC:0.01-0.04 CBDV, 0.005-0.025 CBDVA:0.01-0.03 CBG:0.015-0.04 CBN:0.015-0.04 THCA.

According to a specific embodiment, a concentration ratio of the THC, the CBC, the CBDV, the CBDVA, the CBG, the CBN and/or the THCA in the composition (iii) is 1 THC:0.2-0.4 CBC:0.01-0.03 CBDV, 0.005-0.01 CBDVA:0.01-0.03 CBG:0.015-0.03 CBN:0.015-0.02 THCA.

According to a specific embodiment, a concentration ratio of the THC, the CBC, the CBDV, the CBDVA, the CBG, the CBN and/or the THCA in the composition (iii) is 1 THC:0.3-0.5 CBC:0.02-0.04 CBDV, 0.01-0.025 CBDVA:0.02-0.03 CBG:0.03-0.04 CBN:0.02-0.04 THCA.

According to a specific embodiment, the composition (iii) comprises the phytocannabinoids listed in the F7 composition of Table 1.

According to a specific embodiment, the composition (iii) comprises percentages of phytocannabinoids as listed in the F7 composition of Table 1±10%.

Alternatively or additionally and as mentioned there is provided a composition comprising phytocannabinoids, wherein the phytocannabinoids comprise at least 80% tetrahydrocannabinol (THC) and at least three phytocannabinoids selected from the group consisting of cannabichromene (CBC), cannabigerol (CBG), cannabinol (CBN), tetrahydrocannabinolic acid (THCA) and tetrahydrocannabivarin (THCV), According to a specific embodiment, the composition (iv) comprises at least 80%, 81%, 82%, 83%, 84%, 85% 86%, 87%, 88%, 89%, 90%, 91%, 92%, 93%, 94%, 95%, 96%, 97%, 98%, 99%, 99.5% THC and up to 99.9% THC.

According to a specific embodiment, the composition (iv) comprises at least 80%, 81%, 82%, 83%, 84%, 85% 86%, 87% THC and up to 87% THC.

According to a specific embodiment, the composition (iv) comprises at least 80%, 81%, 82%, 83%, 84% THC and up to 85% THC.

According to a specific embodiment, the composition (iv) comprises at least 4 of the phytocannabinoids.

According to a specific embodiment, the composition (iv) comprises all of the phytocannabinoids.

According to a specific embodiment, the composition (iv) comprises at least 3% of each of the CBC and/or the CBG.

According to a specific embodiment, the composition (iv) comprises at least 4% of each of the CBC and/or the CBG.

According to a specific embodiment, the composition (iv) comprises at least 5% of each of the CBC and/or the CBG.

According to a specific embodiment, the composition (iv) comprises at least 1% of each of the CBN and/or the THCV.

According to a specific embodiment, the composition (iv) further comprises at least one of cannabidiol (CBD) cannabidivarin (CBDV), cannabidivarinic acid (CBDVA), and Cannabigerolic acid (CBGA).

According to a specific embodiment, the composition (iv) further comprises cannabidiol (CBD) cannabidivarin (CBDV), cannabidivarinic acid (CBDVA), and cannabigerolic acid (CBGA).

According to a specific embodiment, the composition (iv) comprises less than 0.5% of each of the CBD, CBDV, CBDVA and CBGA.

According to a specific embodiment, the composition (iv) comprises less than 0.4%, 0.3%, 0.2%, 0.1% of each of the CBD, CBDV, CBDVA and CBGA.

According to a specific embodiment, a concentration ratio of the THC, the CBC, the CBG, the CBN, the THCA, the THCV, the CBD, the CBDV, the CBDVA and/or the CBGA in the composition (iv) is 1 THC:0.04-0.07 CBC:0.03-0.06 CBG, 0.01-0.03 CBN:0.004-0.007 THCA:0.005-0.02 THCV:0.0001-0.002 CBD:0.0001-0.002 CBDV:0.0001-0.002 CBDVA:0.0001-0.002 CBGA.

According to a specific embodiment, the composition (iv) comprises the phytocannabinoids listed in the crude extract composition of Table 1.

According to a specific embodiment, the composition (iv) comprising percentages of phytocannabinoids as listed in the crude extract composition of Table 1±10%.

In certain embodiments, composition (i) is referred to as “F4”; composition (ii) is referred to as “F5” or “F6”; composition (iii) is referred to as “F7”; and composition (iv) is referred to as DQ full extract.

Any of Tables 1, 2 and which are listed in the Examples section of the instant document are to be considered as an integral part of the present specification.

According to other specific embodiments, the composition comprises active compounds other than cannabinoids.

Thus, any of the compositions described herein may comprise cannabis derived components other than cannabinoids, e.g., terpenes, e.g., as listed in Table 2.

Thus, according to specific embodiments, less than 50%, less than 40%, less than 30%, less than 20% or less than 10% of the active compounds in the composition are cannabinoids.

According to a specific embodiment, 5-10% of the active compounds in the composition are cannabinoids.

According to specific embodiments, at least 50% of the active compounds in the composition are cannabinoids.

According to specific embodiments, at least 55%, at least 60% or at least 65%, at least 70%, at least 75% of the active compounds in the composition are cannabinoids.

According to specific embodiments, 50-100%, 60-90% or 60-80% of the active compounds in the composition are cannabinoids.

According to specific embodiments, 90-100% of the active compounds in the composition are cannabinoids.

According to specific embodiments, 95-100% of the active compounds in the composition are cannabinoids.

According to specific embodiments, all the active compounds in the composition are cannabinoids.

The composition of some embodiments of the invention may be a synthetic composition, a compositions comprising purified cannabinoids or a fraction of a cannabis extract.

According to specific embodiments, the composition a cannabis extract (e.g. crude extract not subjected to fractionation).

According to specific embodiments, the composition is not a cannabis extract.

According to specific embodiments, the composition contains 2-30 cannabinoids.

According to specific embodiments, the composition contains 2-25 cannabinoids.

According to specific embodiments, the composition contains 2-20 cannabinoids.

According to specific embodiments, the composition contains 2-15 cannabinoids.

According to specific embodiments, the composition contains 2-10 cannabinoids.

According to specific embodiments, the composition contains 2-7 cannabinoids.

According to specific embodiments, the composition contains 2-6 cannabinoids.

According to specific embodiments, the composition contains 3-25 cannabinoids.

According to specific embodiments, the composition contains 3-20 cannabinoids.

According to specific embodiments, the composition contains 3-15 cannabinoids.

According to specific embodiments, the composition contains 3-10 cannabinoids.

According to specific embodiments, the composition contains 2-7 cannabinoids.

According to specific embodiments, the composition contains 3-6 cannabinoids.

According to specific embodiments, the composition is characterized by increased stability, increased bioavailability, less side effects and/or better pharmacokinetic properties as compared to a cannabis extract.

According to specific embodiments, the composition is a synthetic composition.

Herein, the term “synthetic composition” refers to a chemically defined composition which can include active ingredients which are chemically synthesized and/or purified to a level of purity of at least 99%.

As used herein “a chemically defined composition” refers to a composition in which all the constituents are known by structure and optionally concentration.

According to specific embodiments, the cannabinoids are purified from cannabis.

According to specific embodiments, the cannabinoids are synthetic cannabinoids.

According to specific embodiments, the composition is a cannabis derived fraction.

As used herein “a fraction” refers to a portion of the extract that contains only certain chemical ingredients of the extract but not all.

According to specific embodiments, the composition is a liquid chromatography fraction of a cannabis extract.

According to specific embodiments, the liquid chromatography comprises high pressure liquid chromatography (HPLC) or flash chromatography.

According to specific embodiments, the liquid chromatography fraction of cannabis extract comprises a liquid chromatography pooled fractions of cannabis extract comprising active ingredients detectable by a detector operated at 220 and 280 nm, wherein the active ingredients comprise the compounds disclosed herein.

According to specific embodiments, the liquid chromatography fraction is obtainable by subjecting the cannabis extract to flash chromatography comprising a Flash chromatography apparatus equipped with a diode array detector, a C18 functionalized column, a 55 or 75% to 100% methanol in water gradient at a flow rate of 30 or 60 ml/min. According to specific embodiments, the fraction of composition (i) F4 is collected between about 6-8 minutes (for 60 ml/min) and between 20-25 minutes (for 30 ml/min) of the flash chromatography.

According to specific embodiments, the fraction of composition (ii) F5 is collected between about 8-9 minutes (for 60 ml/min) and between 25-30 minutes (for 30 ml/min) of the flash chromatography.

According to specific embodiments, the fraction of composition (iii) F6 is collected between about 9-12.5 minutes (for 60 ml/min) and between 30-32 minutes (for 30 ml/min) of the flash chromatography.

According to specific embodiments, the fraction of composition (iv) F7 is collected between about 12.5-14 minutes (for 60 ml/min) and between 32-39 minutes (for 30 ml/min) of the flash chromatography.

According to specific embodiments, the detector is a diode array detector.

According to specific embodiments, the liquid chromatography fraction is obtainable by subjecting the cannabis ethanol extract to flash chromatography comprising a Flash chromatography apparatus equipped with a diode array detector, a C18, functionalized column, a 75% to 100% methanol gradient at a flow rate of 60 ml/min.

According to a specific embodiment, the methanol suspended dried ethanol extract is subjected to decarboxylation prior to fractionation process by a flash chromatography apparatus equipped with a diode array detector.

According to a specific embodiment an Ecoflex C-18 80 g column is used for separation, with gradient of methanol and water as the mobile phase, starting with 75% up to 100% methanol.

According to a specific embodiment, detection wavelengths are set at 220 and 280 nm and UV scan monitored from 200-400 nm. Based on the signal intensities of these two wavelengths, the system automatically collected fractions in fraction collector vials. Eluted fractions vials are divided into different fractions based on the identified peaks. Following collection of the fractions the organic solvent (methanol) is removed using a rotary vacuum evaporator.

A non-limiting example of liquid chromatography fractionation that can be effected with specific embodiments of the invention is described in the Examples section which follows, which serves as an integral part of the specification.

Cannabis is a genus of flowering plants in the family Cannabaceae that includes three different species, Cannabis sativa, Cannabis indica and Cannabis ruderalis. The term Cannabis encompasses wild type Cannabis and also variants thereof, including cannabis chemovars which naturally contain different amounts of the individual cannabinoids. For example, some Cannabis strains have been selectively bred to produce high or low levels of THC or CBD and other cannabinoids.

According to specific embodiments, the Cannabis plant is a wild-type plant.

According to specific embodiments, the Cannabis plant is transgenic.

According to specific embodiments, the Cannabis plant is genomically edited.

According to specific embodiments, the Cannabis plant is Cannabis sativa (C. sativa).

According to specific embodiments, the Cannabis plant is C. sativa strain is, Dairy Queen (DQ, IMC, Israel).

The extract may be derived from a cultivated Cannabis plant (i.e. not grown in their natural habitat) or may be derived from Cannabis plants which grow in the wild.

The tissue of the Cannabis plant from which the extract is typically obtained is the inflorescence. Accordingly, the extract may be obtained from the complete flower head of a plant including stems, stalks, bracts, and flowers. However, it will be appreciated that a cannabis extract of some embodiments the invention may be obtained from only part of the inflorescence, such as from the bracts and/or flowers.

A non-limiting example of growing the plant and obtaining the extract, which can be used with specific embodiments of the invention, is described in the Examples section which follows, which serve as an integral part of the specification.

According to specific embodiments, the extract is obtained from a fresh plant (i.e. a plant not heated prior to the extraction process). Fresh plants include plants taken immediately following harvesting (e.g., up to an hour or several hours) for extraction as well as plants frozen immediately after harvesting (e.g. at about −70° C. to −90° C., e.g. at −80° C., for any required length of time) prior to extraction.

According to specific embodiments, the extract is obtained from fresh inflorescence.

According to specific embodiments, the extract is obtained from a frozen inflorescence (e.g. frozen immediately after harvesting at about −70° C. to −90° C., e.g. at −80° C., for any required length of time). Thus, for example, the extract may be obtained from a cryopreserved inflorescence, or from an inflorescence frozen in liquid nitrogen or in dry ice.

According to specific embodiments, the extract is obtained from an inflorescence which has not been subjected to heating (such as heating at e.g. at 120° C. to 180° C., e.g. at 150° C., for any length of time, such as for 1-5 hours).

According to specific embodiments, the extract is obtained from dry Cannabis inflorescence. Drying the inflorescence may be carried out using any method known in the art, such as by pulverizing with liquid nitrogen or with dry-ice/alcohol mixture.

According to specific embodiments, the dry inflorescence is obtained from the grower.

According to specific embodiments, the polar solvent comprises a polar, protic solvent (e.g., ethanol or methanol). In some embodiments, the polar solvent comprises a polar, aprotic solvent (e.g., acetone). Polar solvents suitable for use with specific embodiments of the present invention include, but are not limited to, ethanol, methanol, n-propanol, iso-propanol, a butanol, a pentanol, acetone, methylethylketone, ethylacetate, acetonitrile, tetrahydrofuran, dimethylformamide, dimethylsulfoxide, water, and combinations thereof.

According to specific embodiments, the polar solvent is ethanol (e.g. absolute ethanol i.e. above 99.8%, or in the range of 99-70% in water).

The concentration or amount of a polar solvent used to Cannabis inflorescence can be varied. Generally, the ratio of a Cannabis inflorescence to a polar solvent (weight to volume) is the amount of a polar solvent sufficient to extract about 70% or more, about 75% or more, about 85% or more, about 90% or more, about 95% or more, about 97% or more, or about 99% or more of a composition having a cytotoxic activity. In some embodiments, the ratio of polar solvent to Cannabis inflorescence is about 1:2 to about 1:20 (w/v), e.g. about 1:4 to about 1:10 (w/v).

In particular embodiments, the extract is an ethanol extract.

In particular embodiments, absolute ethanol is added to the inflorescence at a sample-to-absolute ethanol ratio of 1:4 (w/v).

In some embodiments, the Cannabis inflorescence is contacted with a polar solvent (e.g. ethanol) for about 15 minutes or more, about 30 minutes or more, about 1 hour or more, about 2 hours or more, or about 5 hours or more.

According to specific embodiments, the Cannabis inflorescence is contacted with a polar solvent (e.g. ethanol) for about 30 minutes.

Temperature can also be controlled during the contacting. In some embodiments, the Cannabis inflorescence is contacted with a polar solvent at temperature of about 15° C. to about 35° C., or about 20° C. to about 25° C.

According to specific embodiments, the Cannabis inflorescence is contacted with a polar solvent (e.g. ethanol) while being constantly mixed e.g. on a shaker.

In some embodiments, the process of obtaining the composition of some embodiments of the present invention comprises isolating a liquid extract (i.e. filtered extract) from the mixture (i.e. crude extract) comprising the liquid extract and solids. Suitable means for isolating the liquid extract (i.e. filtered extract) include those known in the art of organic synthesis and include, but are not limited to, gravity filtration, suction and/or vacuum filtration, centrifuging, setting and decanting, and the like. In some embodiments, the isolating comprises filtering a liquid extract through a porous membrane, syringe, sponge, zeolite, paper, or the like having a pore size of about 1-5 μm, about 0.5-5 μm, about 0.1-5 μm, about 1-2 μm, about 0.5-2 μm, about 0.1-2 μm, about 0.5-1 μm, about 0.1-1 μm, about 0.25-0.45 μm, or about 0.1-0.5 μm (e.g. about 2 μm, about 1 μm, about 0.45 μm, or about 0.25 μm).

According to a specific embodiment, the crude extract is filtered through a 0.45-μm syringe filter such as that commercially available from Merck, Darmstadt, Germany.

According specific embodiments, process of obtaining the composition of some embodiments of the present invention comprises drying (i.e. removal of the polar solvent) and/or freezing the filtered extract following generation thereof.

The method for drying the filtered extract (i.e. removing the polar solvent) is not particularly limited, and can include solvent evaporation at a reduced pressure (e.g., sub-atmospheric pressure) and/or an elevated temperature (e.g., above about 25° C.). In some embodiments, it can be difficult to completely remove a polar solvent from a liquid extract by standard solvent removal procedures such as evaporation. In some embodiments, processes such as co-evaporation, lyophilization, and the like can be used to completely remove the polar solvent from a liquid fraction to form a dry powder, dry pellet, dry granulate, paste, and the like. According to a specific embodiment, the polar solvent is evaporated with a vacuum evaporator.

According to specific embodiments, the extract (e.g. the filtered extract) is subjected to a decarboxylation step. Decarboxylation may be effected by heating the extract in a pressure tube in the oven at 220° C. for 10 minutes.

Following generation of the filtered extract, specific embodiments of the process of obtaining the composition of some embodiments of the present invention comprises additional purification steps so as to further purify active agents from the extract.

Thus, for example, fractionating the filtered extract. Fractionating can be performed by processes such as, but not limited to: column chromatography, preparative high performance liquid chromatography (“HPLC”), flash chromatography, reduced pressure distillation, and combinations thereof.

According to a specific embodiment, fractionating is performed by HPLC or flash chromatography.

In some embodiments, fractionating comprises re-suspending the filtered extract in a polar solvent (such as methanol, as discussed above), applying the polar extract to a separation column, and isolating the Cannabis fraction by column chromatography (e.g. preparative HPLC, flow cytometry).

An eluting solvent is applied to the separation column with the polar extract to elute fractions from the polar extract. Suitable eluting solvents for use include, but are not limited to, methanol, ethanol, propanol, acetone, acetic acid, carbon dioxide, methylethyl ketone, acetonitrile, butyronitrile, carbon dioxide, ethyl acetate, tetrahydrofuran, di-iso-propylether, ammonia, triethylamine, N,N-dimethylformamide, N,N-dimethylacetamide, and the like, and combinations thereof.

According to an alternative or an additional embodiment, liquid chromatography is performed on a reverse stationary phase.

According to an alternative or an additional embodiment, liquid chromatography comprises high performance liquid chromatography (HPLC) or flash chromatography, as further described hereinabove.

The fractions or extract obtained may be immediately used or stored until further use.

According to specific embodiments, the fraction or extract is kept frozen, e.g. in a freezer, until further use (e.g. at about −20° C. to −90° C., at about −70° C. to −90° C., e.g. at −80° C.), for any required length of time.

According to other specific embodiments, the fraction or extract is immediately used (e.g. within a few minutes e.g., up to 30 minutes).

The extracts and/or fractions may be used separately. Alternatively, different extracts (e.g. from different plants or from separate extraction procedures) may be pooled together. Likewise, different fractions (from the same extract, from different extracts, from different plants and/or from separate extraction procedures) may be pooled together.

The term “pooled” as used herein refers to collected from the liquid chromatography (e.g. HPLC, flash chromatography) either as a single fraction or a plurality of fractions.

The compositions of some embodiments of the invention have an anti-cancer effect on cancer cells e.g., brain tumor or ovarian cancer.

According to specific embodiments, the composition has a combined additive or synergistic anti-cancer effect on cancer cells as compared to each of the recited cannabinoids (e.g. CBG, CBN, THC, THCV) when administered as a single agent.

According to specific embodiments, the composition has anti-cancer activity on Glioblastoma multiforme (GMB) cells and/or ovarian cancer cells.

According to specific embodiments, the composition has a combined synergistic anti-cancer activity as compared to each of the phytocannabinoids comprised in the composition when administered as a single agent.

Thus, according to an aspect of the invention there is provided a method of treating cancer in a subject in need thereof, the method comprising administering to the subject a therapeutically effective amount of the composition as described herein, thereby treating the cancer in the subject.

According to an additional or an alternative aspect, there is provided a method of reducing viability, inducing cell cycle arrest and/or reducing proliferation and/or migration of a cancerous cell, the method comprising contacting the cancerous cell with the composition as described herein.

According to some embodiments, the contacting is effected ex-vivo or in-vitro.

According to some embodiments, the contacting is effected in-vivo.

According to specific embodiments, the anti-cancer activity is manifested by reduced viability, cell cycle arrest and/or reduced proliferation and/or migration.

According to specific embodiments, the anti-cancer activity is manifested by expression of genes associated with ER stress, reduced viability of glioma stem-like cells (GSCs), reduced motility and invasion, disintegration of F-actin and/or reduced colony/neurosphere formation, cell cycle distribution, cell killing, MAPK4 signaling.

As used herein the term “anti-cancer” refers to a statistically significant decrease in cancer growth and/or invasiveness in the presence of the composition in comparison to same in the absence of the composition. Such an effect may be manifested by, for example, but not limited to, reduced viability of cancer cells (e.g. ovarian or brain cancer cells), induction of cancer cell cycle arrest, reduced migration of cancer cells (e.g. ovarian or brain cancer cells), inhibition of sphere formation of cancer cells (e.g. ovarian or brain cancer cells), inhibition of epithelial to mesenchymal transition of cancer cells (e.g. ovarian or brain cancer cells). Alternatively or additionally, the anti-cancer effect may be manifested by improvement of one or more of the various physiological symptoms associated with cancer in a subject in need e.g. increased survival rate, increased progression without disease, decreased tumor size, decreased metastasis and the like.

According to a specific embodiment, the decrease is in at least 2%, 5%, 10%, 30%, 40% or even higher say, 50%, 60%, 70%, 80%, 90% or 100% as compared to same in the absence of the composition.

According to specific embodiments, the decrease is at least 1.5 fold, at least 2 fold, at least 3 fold, at least 5 fold, at least 10 fold, or at least 20 fold as compared to same in the absence of the composition.

Methods of determining an anti-cancer effect are well known in the art and are also described hereinbelow and in the Examples section which follows.

Non-limiting examples of methods of determining cell viability or cytotoxicity include the XTT assay, Annexin V assay [MEBCYTO Apoptosis Kit or ApoAlert® Annexin V Apoptosis Kit (Clontech Laboratories, Inc., CA, USA)]; the Senescence associated-β-galactosidase assay (Dimri G P, Lee X, et al. 1995. Proc Natl Acad Sci USA 92:9363-9367); MTT test which is based on the selective ability of living cells to reduce the yellow salt MTT (3-(4,5-dimethylthiazolyl-2)-2,5-diphenyltetrazolium bromide) (Sigma, Aldrich St Louis, MO, USA) to a purple-blue insoluble formazan precipitate; the BrDu assay [Cell Proliferation ELISA BrdU colorimetric kit (Roche, Mannheim, Germany]; the TUNEL assay [Roche, Mannheim, Germany]; the as well as various RNA and protein detection methods (which detect level of expression and/or activity).

According to specific embodiments, the composition is capable of inducing apoptosis of cancer cells e.g. ovarian or brain cancer cells.

According to specific embodiments, the composition is capable of inducing necrosis of cancer cells e.g. ovarian or brain cancer cells.

A non-limiting example of a method of determining cell cycle arrest includes flow cytometry following PI staining.

Non-limiting examples of determining migration include the scratch assay, the transwell assay, cytoskeleton staining.

According to specific embodiments, the composition is capable of inducing disintegration of F-actin filaments in cancer cells e.g. ovarian or brain cancer cells.

According to specific embodiments, the composition is capable of inhibiting cancer cells e.g. ovarian or brain cancer cells sphere formation.

Methods of determining sphere formation are well known in the art and are further described in the Examples section which follows.

Consequently, according to an aspect of the present invention, there is provided a method of reducing viability, inducing cell cycle arrest and/or reducing migration of a cancerous cell, the method comprising contacting the cancerous cell with the composition disclosed herein.

According to specific embodiments, the contacting is effected in-vitro or ex-vivo.

According to other specific embodiments, the contacting is effected in-vivo.

According to specific embodiments, the method comprises determining the anti-cancer effect.

As the compositions disclosed herein are endowed with anti-cancer effects, specific embodiments suggest their use in treating cancer in a subject in need.

Compositions (extracts or parts thereof) alone or in combination with other active ingredients can be used in therapy, such as for the treatment or prevention of cancer.

As used herein, the term “subject” or “subject in need thereof” includes mammals, preferably human beings at any age or gender which suffer from the pathology i.e. cancer. According to specific embodiments, this term encompasses individuals who are at risk to develop the pathology.

According to specific embodiments, the subject is diagnosed with cancer.

As used herein the term “treating” refers to curing, reversing, attenuating, alleviating, minimizing, suppressing or halting the deleterious effects of a disease or disorder (e.g. disease that can benefit from activating macrophages or cytokine storm). Those of skill in the art will understand that various methodologies and assays can be used to assess the development of a pathology, and similarly, various methodologies and assays may be used to assess the reduction, remission or regression of a pathology (e.g. a malignancy).

According to a specific embodiment, treating is preventing.

As used herein, the term “preventing” refers to keeping a disease, disorder or condition from occurring in a subject who may be at risk for the disease, but has not yet been diagnosed as having the disease.

Non-limiting examples of cancers which can be treated by the method of this aspect of some embodiments of the invention can be any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to a specific embodiment, the cancer is ovarian cancer.

According to a specific embodiment, the cancer is glioblastoma (GBM).

According to specific embodiments, the cancer is not urothelial cancer.

Each of the compositions or fractions described herein can be administered to an organism per se, or in a pharmaceutical composition where it is mixed with suitable carriers or excipients. In some case the combination of say more than one composition (e.g., a fraction or synthetic versions thereof) optionally with another anti cancer agent, e.g., chemotherapy are either co-formulated or each is formulated in a pharmaceutical composition which can be marketed as a kit for instance.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cannabis derived active ingredients e.g. phytocannabinoids or synthetic analogs thereof accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as an oil-based formulation, tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

The pharmaceutical composition can be formulated for inhalation. For example, the compositions can be formulated as vapors or aerosols that can be inhaled into the lungs. Vapor formulations include liquid formulations that are vaporized when loaded into a suitable vaporization device.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For transdermal administration, the composition can be formulated in a form of a gel, a cream, an ointment, a paste, a lotion, a milk, a suspension, an aerosol, a spray, a foam, a serum, a swab, a pledget, a pad or a patch. Formulations for transdermal delivery can typically include carriers such as water, liquid alcohols, liquid glycols, liquid polyalkylene glycols, liquid esters, liquid amides, liquid protein hydrolysates, liquid alkylated protein hydrolysates, liquid lanolin, lanolin derivatives, glycerin, mineral oil, silicone, petroleum jelly, lanolin, fatty acids, vegetable oils, parabens, waxes, and like materials commonly employed in topical compositions. Various additives, known to those skilled in the art, may be included in the transdermal formulations of the invention. For example, solvents may be used to solubilize certain active ingredients substances. Other optional additives include skin permeation enhancers, opacifiers, anti-oxidants, gelling agents, thickening agents, stabilizers, and the like.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran.

Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (cannabis derived active ingredients) effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

A non-limiting example of an animal model for SARS-CoV-2 is the transgenic mouse expressing human ACE2 (see e.g, Bao et al. (2020) Nature 583: 830-833.

The doses determined in the mouse animal model can be converted for the treatment other species such as human and other animals diagnosed with the disease, using conversion Tables known to the skilled in the art.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

As mentioned, any of the compositions described herein can be provided in combination with at least one other anti cancer agent that can be, for example, a small molecule chemical (e.g., chemotherapy), a biological molecule (e.g., antibody, polypeptide, nucleic acid molecule, e.g., DNA, RNA or combination of same) or a physical treatment, e.g., radiation.

According to specific embodiments, the anti-cancer agent is a chemotherapy. Typically, chemotherapy agents that can be used according to the present teachings include, but are not limited to, alkylating agents, nitrosoureas, anti metabolites, anti tumor antibiotics, topoisomerase inhibitors, mitotic inhibitors or corticosteroids. Other types of chemotherapy include but are not limited to, all-trans-retinoic acid, arsenic trioxide, asparaginase, eribulin, hydroxyurea, Ixabepilorie, mitotane, omacetaxine, pegaspargase, procarbazine, rornidepsin and vorinostat.

According to a specific embodiment, the small molecule is a PARP inhibitor, e.g., olaparib or niraparib.

According to specific embodiments, the anti-cancer agent is an anti-solid tumor cancer.

According to a specific embodiment, the chemotherapy is for a brain tumor, e.g., glioblastoma, such as temozolomide, the current “gold standard” of care, carmustine, another common medication for high-grade brain cancers; bevacizumab, typically used as a second-line treatment for recurrent glioblastomas; and lomustine, which may help improve the efficacy of bevacizurnab when both medications are administered at the same time.

According to a specific embodiment, the chemotherapy is for ovarian cancer, e.g., paclitaxel or platin-based chemotherapy e.g., cisplatin or carboplatin.

According to a specific embodiment, the anti cancer agent is against a ovarian tumor (e.g., glioma or glioblastoma) or an anti female cancer such as an ovarian cancer.

Non-limiting examples of anti-cancer agents that can be used with specific embodiments of the invention include niraparib or platinum based chemotherapy such as, but not limited to cisplatinum, carboplatinum.

According to a specific embodiment, an additive effect or synergy is particularly envisaged for a combination of cisplatin or niraparib with any of:
a composition comprising phytocannabinoids, wherein the phytocannabinoids comprise at least 85% tetrahydrocannabinol (THC) and at least 1.5% cannabigerol (CBG);
a composition comprising phytocannabinoids, wherein the phytocannabinoids comprise at least 50% tetrahydrocannabinol (THC), at least 10% cannabichromene (CBC) and at least one phytocannabinoid selected from the group consisting of cannabidivarin (CBDV), cannabidivarinic acid (CBDVA), cannabigerol (CBG), cannabinol (CBN) and tetrahydrocannabinolic acid (THCA);
a composition comprising the phytocannabinoids listed in the F5 composition of Table 1 in percentages as listed in the F5 composition of Table 1±10% or a composition comprising the phytocannabinoids listed in the F6 composition of Table 1 in percentages as listed in the F6 composition of Table 1±10%; and
(iii) a composition comprising the phytocannabinoids listed in the F7 composition of Table 1 in percentages as listed in the F7 composition of Table 1±10%.

According to a specific embodiment the combination is niraparib with a composition comprising the phytocannabinoids listed in the F7 composition of Table 1 in percentages as listed in the F7 composition of Table 1±10%.

According to a specific embodiment, the anti-cancer agent is an antibody.

Various antibodies are known for the treatment of cancer and more will be developed, each of which is envisaged according to the teachings of the invention.

A list of FDA approved antibodies for the treatment of cancer is provided in the following citation Baldo, B. A. (2016). Monoclonal Antibodies Approved for Cancer Therapy. In: Safety of Biologics Therapy, Springer. Cham, www(dot)/doi(dot)org/10.1007/978-3-319-30472-4_3, which is hereby incorporated by reference.

According to a specific embodiment, the antibody is against VEGF-A, e.g., Bevacizumab branded as Avastin™.

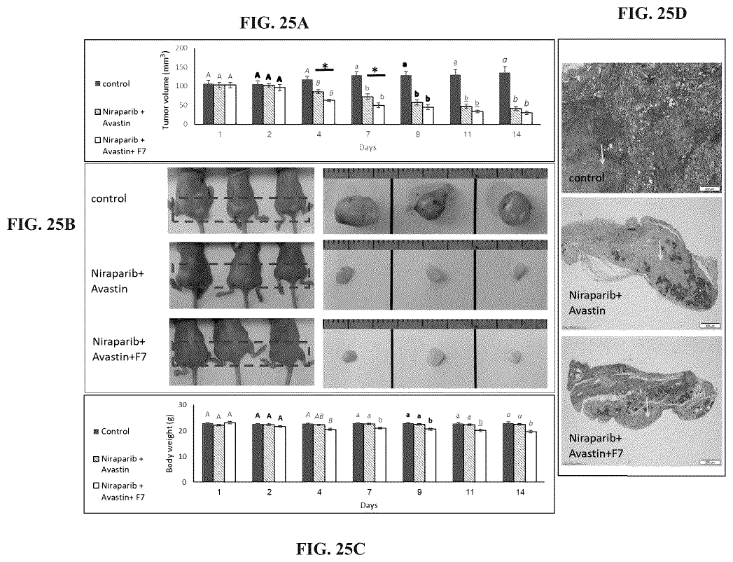

According to a specific embodiment, a combination of anti VEGF-A (e.g., Bevacizumab) and a composition comprising the phytocannabinoids listed in the F7 composition of Table 1 in percentages as listed in the F7 composition of Table 1±10%. Optionally the combination comprises niraparib. Such combinations may be of particular value in the treatment of ovarian cancer such as shown in FIGS. 25A-D.

According to specific embodiments, the anti-cancer agent is an immune-modulatory agent.

Immune-modulatory agents are well known in the art and include, but not limited to, chemokine receptor modulators, immune-check point modulators and cytokines.

Examples of check-point proteins that can be targeted according to specific embodiments of the invention include, but not limited to, PD1, PDL-1, B7H2, B7H3, B7H4, BTLA-4, HVEM, CTLA-4, CD80, CD86, LAG-3, TIM-3, KIR, IDO, CD19, OX40, OX40L, 4-1BB (CD137), 4-1BBL, CD27, CD70, CD40, CD40L, GITR, CD28, ICOS (CD278), ICOSL, VISTA and adenosine A2a receptor.

According to specific embodiments the immune modulatory agent is selected from the group consisting of a PD1 inhibitor, a PDL-1 inhibitor and a CTLA-4 inhibitor.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The compositions disclosed herein can be administered to a subject (e.g., a human) in need thereof in a variety of other forms including a nutraceutical composition.

As used herein, a “nutraceutical composition” refers to any substance that may be considered a food or part of a food and provides medical or health benefits, including the prevention and treatment of disease. In some embodiments, a nutraceutical composition is intended to supplement the diet and contains at least one or more of the following ingredients: a vitamin; a mineral; an herb; a botanical; a fruit; a vegetable; an amino acid; or a concentrate, metabolite, constituent, or extract of any of the previously mentioned ingredients; and combinations thereof.

In some embodiments, a nutraceutical composition of the present invention can be administered as a “dietary supplement,” as defined by the U.S. Food and Drug Administration, which is a product taken by mouth that contains a “dietary ingredient” such as, but not limited to, a vitamin, a mineral, an herb or other botanical, an amino acid, and substances such as an enzyme, an organ tissue, a glandular, a metabolite, or an extract or concentrate thereof.

Non-limiting forms of nutraceutical compositions of the present invention include: a tablet, a capsule, a pill, a softgel, a gelcap, a liquid, a powder, a solution, a tincture, a suspension, a syrup, or other forms known to persons of skill in the art. A nutraceutical composition can also be in the form of a food, such as, but not limited to, a food bar, a beverage, a food gel, a food additive/supplement, a powder, a syrup, and combinations thereof.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Plant extraction—Dry inflorescences of high-THC strain of C. sativa Dairy Queen (DQ) (IMC, Israel), high CBD strain of C. sativa from IMC, super CBD (sCBDm IMC, (Israel), and PARIS (IMC, Israel) were extracted by grounding them using pestle and mortar with liquid nitrogen. The powder transferred into glass tubes and absolute ethanol was added to each tube in a ratio of 1:4 (w/v). The tubes were mixed thoroughly on a shaker for 45 minutes in a speed of 250 rpm and then the extract was filtered through a 0.45 μm syringe filter. The extract was completely dried first by evaporation under dry nitrogen and then by overnight lyophilization to remove water residuals. The dried extract was weighed and resuspended in absolute methanol to the desired concentration. Decarboxylation was effected by heating dry extract to 220° C. for 10 minutes in pressure tubes. Pressure tubes were used in this process to preserve volatile substances. The decarboxylated extract was weighed and re-suspended in methanol to the desired concentrations. All extracts solutions: fresh, decarboxylated or fractions placed in a known weight vial. After evaporation, the vial contained only the dry compound. The vial was weight before and after evaporation so as to calculate the final weight of the dried extract: (vial's weight after evaporation−weight of the empty vial=dry weight); and suspended in methanol to a final concentration of 2 mg/ml.

Extract fractionation—The decarboxylated crude extract solution dissolved in methanol underwent fractionation process by a flash chromatography apparatus equipped with a diode array detector. An Ecoflex C-18 80 g (Flash Pure, Buchi, C-18, 80 μm spherical, max. pressure 180 psi) column was used for separation, with gradient of methanol and water as the mobile phase, starting with 75% up to 100% methanol. Detection wavelengths were set at 220 and 280 nm and UV scan monitored from 200-400 nm. Based on the signal intensities of these two wavelengths, the system automatically collected fractions in fraction collector vials. Eluted fractions vials were divided into different fractions based on the identified peaks. Following collection of the fractions the organic solvent (methanol) was removed using a rotary vacuum evaporator at 30° C. The remaining aqueous phase containing the compound of interest was further lyophilized to precipitate a dried powder. Each dried fraction tube was weighed separately and reconstituted using methanol to produce a solution with the required concentration and stored at −20° C.

Chemical analysis—High performance liquid chromatography (HPLC 1260 Infinity II, Agilent) equipped with a Raptor ARC-18 LC-UV column (150 mm×4.6 mm ID, pore size 2.7 μm) was used for chemical analysis as described in [29]. Isocratic separation was used with acetonitrile (25%) and water with 5 mM ammonium formate, 0.1% formic acid (75%) at a constant flow rate of 1.5 Ml/minute. Sample concentration was 100 μg/mL and injected volume was 5 μL. Cannabinoid profiles and fraction quantification were carried out in comparison to the standard calibration curves obtained by dissolving cannabinoid standards in methanol at a range of concentrations from 0-100 μg/mL. Gas chromatography with mass selective detector (GC/MS 8860 GC/5977B MSD, Agilent) equipped with 30 m, 0.25 mm ID, 5% cross-linked phenylmethyl siloxane capillary column (HP-5MS) with 0.25 μm film thickness used for chemical analysis as described in [29]. 10 μL of each fraction sample was transferred into a GC vial with 0.2 ml conical insert, dried under a gentle stream of nitrogen and dissolved in 100 μL of hexane. Sample volume for injection was 1 μL. Helium was used as the carrier gas at a constant flow of 1.1 mL/s. An isothermal hold at 50° C. was maintained for 2 minutes, followed by a heating gradient of 6° C./min to 300° C., and the final temperature was held for 4 minutes. Peak assignments were performed using spectral libraries (NIST 14.0 and 17.0) and compared with MS data obtained from the injection of purchased standards (LGC Standards). Hence, the output of the HPLC analysis contained the amount ((μg/mL) of each of the phytocannabinoids and other cannabis derived compounds presented in the tested sample.

Standard/material preparation and use—The cannabinoid standards (at a concentration of 1 mg/mL in methanol) used in this study included cannabidiol (CBD, Restek catalog no. 34011), cannabigerol (CBG, Restek catalog no. 34091), tetrahydrocannabivarin (THCV, Restek catalog no. 34100), cannabinol (CBN, Restek catalog no. 34010) and delta-9-tetrahydrocannabidiol (Δ-9 THC, Restek catalog no. 34067). For preparation of the standard mix (SM), the indicated phytocannabinoids were mixed in a concentration equivalent to the concentration in the indicated fraction (see Table 1 hereinbelow) to a final concentration of 1 mg/ml. In order to examine the efficiency of the fraction consisting of standards in comparison with the original plant fraction, the complex fraction was examined in a range of concentrations. from 0-15 μg/mL. Inverse agonists (IA) to CB1 and CB2 used included AM251 (ab120088; Abcam) and SR144528 (ab146185; Abcam), respectively. The transient receptor potential ankyrin subtype 1 protein (TRPA1) blocker used was HC-030031 (ab120554; Abcam). Transient receptor potential vanilloid receptor 1 (TRPV1) and 2 (TRPV2) antagonists were SB-366791 (ab141772-5-B Abcam) and Tranilast (1098/10 Abcam), respectively. All IAs were dissolved in dimethyl sulfoxide (DMSO) at a concentration of 10 mM. Doxorubicin (D1515; Sigma Aldrich, USA) served as positive control in concentrations of 0.5 μg/mL on A172 cells and 50 μg/mL on U87. Temozolomid (TMZ, T2577; Sigma Aldrich, USA) was tested as positive control. Analytical grade methanol was used according to the indicated concentration of the treatment. Ultra-pure deionized water (MS grade) was used as received without further purification.

Cell cultures—Human glioblastoma A172 (ATCC®CRL-1620™) was cultured in DMEM (01-055-1A, Biological Industries, Israel) and U87MG (U87; ATCC®HTB-14™) in EMEM (01-058-1A; Biological Industries, Israel). Both media were supplemented with 10% fetal bovine serum (FBS, 04-127-1A, Biological Industries, Israel), 1% Pen-Strep, 1% L-Glutamine and 0.02% plasmocin (i.e., complete medium). Cells were incubated at 37° C. in a humidified atmosphere containing 5% CO2—95% air. Serum free media (SFM) was composed of DMEM/F12 (01-170-1A; Biological Industries, Israel), including all the supplements above except for FBS. Neurosphere media for A172 was composed of SFM with 2% B-27; Neurosphere media for U87 was serum-free EMEM without B27. For Glioblastoma stem cells (GSCs) cultures all human materials were used in accordance with the policies of the Henry Ford Hospital Institutional Review Board. Generation of GSCs from fresh GBM specimens and their characterization have been recently described [30-31]. The GSCs were plated in neurosphere medium (DMEM-F12 1:1, glutamine 10 mM, HEPES buffer 10 mM and sodium bicarbonate 0.025%) supplemented with EGF and bFGF (20 ng/mL).

Cell viability assays—Cells were seeded in 96-well plates at density of 2×10⁴ per well (100 μL/well) in SFM (for A172) or complete medium (for U87) and were incubated at 37° C. overnight to allow attachment. The following day, cells were treated with plant extracts (n=3), fractions, or cannabinoid standards in volume of 100 μL/well at different concentrations. Solvents used as a vehicle control and doxorubicin was used as a positive control in all the biological assays. Where indicated, CB1 or CB2 inverse agonists, TRPV1 or TRPV2 antagonists or TRPA1 blocker were added along with the indicated treatments at a concentration of 10 μM/mL in complete medium. Treated cells were incubated for 48 h at 37° C. Subsequently, XTT reagents (2,3-bis(2-methoxy-4-nitro-5-sulfophenyl)-5-[(phenylamine)-carbonyl]-2H-tetrazolium inner salt) (20-300-1000; Biological Industries, Israel) were added to the cells for 2 hours at 37° C. in a humidified 5% CO₂—95% air atmosphere. Absorbance was recorded by a Synergy H1 hybrid reader photometer (BioTek) at 490 nm with 650 nm of reference wavelength. Cell viability was estimated from the equation:

$$\% Cell Viability = 100 \times \frac{( A 490 - A 650 ) of treatment}{( A 490 - A 650 ) of solvent contro}$$

A490 and A650 are the absorbencies of the XTT colorimetric reaction. Absorbance of medium/solvent alone (blank) was subtracted from the readings.

For dose response assays, data points were connected by non-linear regression lines of the sigmoidal dose-response relation. GraphPad Prism version 6.1 (www(dot)graphpad(dot)com/scientific-software/prism/, GraphPad Software Inc., San Diego, USA) was employed to produce dose-response curves and determination of IC50 values.

For GSCs cell viability was quantified by counting cells with trypan blue exclusion assay and using the lactate dehydrogenase cytotoxicity (LDH) assay kit. GSC neuro-spheres were disaggregated and 10⁵/mL cells were plated in triplicates in 24-well plates. The cells were treated with the specific compounds and cell death was determined at 24 and 48 hours using the LDH assay kit according to the manufacturer's instructions and as previously described [32]. The absorbance value of each sample was read at 490 nm, and cell death was determined compared to control untreated cells.

Colony forming assay—A172 or U87 cells were seeded in a 6-well plate at a density of 5×10⁵ in 3 mL of SFM or EMEM and were incubated at 37° C. overnight to allow attachment. The following day, cells were treated with the indicated treatments in complete medium. Following 24 hours of incubation, cells were washed twice with 2 mL of PBS, harvested and centrifuged for 3 minutes at 1400 rpm, then re-plated in serial cell-density dilutions in neurosphere media for 48 hours incubation. Colonies were imaged and counted using an inverted microscope (Primo Vert Zeiss). Groups of more than 20 cells were identified as a colony [33]. Percentage of colony in the different treatments was calculated out of average number of colonies in the control at the highest cell concentration (6×10⁴).

Scratch-wound assay—A172 cells were seeded in a 96-well plate at a density of 2×10⁴ per well in 100 μL of complete medium. Following 24 hours, cells in each well were scratched perpendicularly across the center of the well with a 200 μL pipette tip to produce a cell-free area for investigating the ability of the cells to migrate and close the gap under different treatments. Immediately following scratching, 100 μL of treatment solution was added. Photos were taken by a CARL ZEISS inverted microscope coupled with a camera at 0, 14, 20, and 36 hours following scratching, and the gap area was measured using ImageJ (version 1.53a). The scratch area, indicated by cells migrated into the scratch was calculated as percent of scratch area at time x from time 0:

$$\frac{( z h cell free area ) \times 100}{( 0 h cell free area )}$$

Transwell assay—Cells were seeded in the upper chamber of a 24-well plate containing insert with an 8-μm pore size membrane (BD, Falcon Cat #353097), at a density of 5×10⁴ in 250 μL complete medium and were incubated at 37° C. for 60 minutes to recover. Following, cells were treated and incubated for 24 hours followed by viability test with resazurin (AR002, R&D Systems, USA). The inserts were taken out of the medium, the inner part was wiped using a cotton swab to remove the detached cells. The inserts were transferred into a new 24-well plate, fixated using 70% ethanol for 10 minutes and dried for 50 minutes, followed by staining in 400 μL of 0.2% crystal violet, washing with PBS, and drying for 5 minutes. Cells stained underneath the membrane were counted in inverted microscope in 3-5 different fields.

Cell staining—EasyProbes™ ActinRed 555 Stain was used for F-actin staining and Hoechst 33342 (ABP Biosciences, USA) for nuclei staining, according to the manufacturer's instructions. Briefly, cells were seeded on confocal dishes (D35-20-1.5-N, Cellvis, USA) at a density of 2×10⁴ in 500 μL complete medium. Following 24 hours incubation, the indicated treatments at different concentrations were added for additional 24 hours. For the staining process, cells were washed with PBS and fixed with 3.7% formaldehyde solution in PBS and incubated at room temperature for 10 minutes followed by incubation in 0.1% Triton X-100 (T8787; Sigma Aldrich, USA) for 5 minutes, washing with PBS and incubation in 1% BSA (A7284; Sigma Aldrich, USA) solution for 30 minutes. Two drops of EasyProbes™ Actin and of Hoechst were applied to each of the samples for 20 minutes. Image acquisition was carried out using a Leica SP8 laser scanning microscope (Leica, Wetzlar, Germany), equipped with a diode laser with 405 nm and OPSL 552 nm laser, HC PL APO CS 10×/0.40 and HC PL APO CS2 63×/1.20 objectives (Leica, Wetzlar, Germany) and Leica Application Suite X software (LASX, Leica, Wetzlar, Germany). The number of F-actin filaments was quantified as the number of filaments that crosse a line that is drawn electronically across the soma. Percentage of filaments in the different treatments was calculated out of the average number of filaments in the control.

Apoptosis assay—Apoptosis for the A172 cell line was assessed using MEBCYTO Apoptosis Kit with Annexin V-FITC and PI (MBL, Enco, 4700). Staining was carried out according to manufacturer's instructions. In brief, cells were seeded in a 6-well TC plate, at a density of 4×10⁵ cells in 2 mL of SFM per well. 24 hours following seeding, cells were treated with the indicated treatments in complete medium for 48 hours. Following incubation, cells were harvested using trypsin and centrifuged for 5 minutes at 1600 rpm. Cell pellets were resuspended and washed twice with 1 mL of PBS. The cells in each sample were resuspended in 85 μL of Annexin binding buffer. Cells were stained using 10 μL of Annexin V-FITC solution and 5 μL of propidium iodide (PI) working solution followed by incubation in the dark at room temperature for 15 minutes. Then 400 μL of Annexin V binding buffer was added to each tube and flow cytometry was performed using a Gallios flow cytometer (FACS). Cells were considered apoptotic if they were Annexin V+/PI− (early apoptosis) or Annexin V+/PI+ (late apoptosis). Live cells were defined as Annexin V−/PI−, and Annexin V−/PI+ as necrosis.

Cell cycle analysis—A172 cells were seeded in 6-well TC plate at a concentration of 4×10⁵ cells in 2 mL of SFM per well. Following 24 hours incubation, cells were treated with indicated treatments in complete medium for 24 hours. Methanol and doxorubicin were used as negative and positive controls, respectively. Cells were harvested and centrifuged for 5 minutes at 1600 rpm. Cell pellets were washed once with 1 mL of PBS and fixed with 70% cold ethanol overnight at −20° C. The fixed cells were washed twice with 1 mL of PBS and stained with 250 μL of PI solution (50 μg/mL) containing RNase A (100 μg/mL) for 30 minutes in dark conditions. 250 μL PBS was added to each tube and the cells were analyzed using FACS.

Quantitative real-time PCR—Cells were seeded in a 6-well plate at a concentration of 1×10⁶ cells in 5 mL of SFM per well. Following 24 hours incubation, cells were treated with the indicated treatments in complete medium. Cells were harvested 4, 12 or 24 hours following treatment and RNA was extracted using TRI reagent (T9424; Sigma Aldrich, USA). RNA was reverse-transcribed in a total volume of 20 μL (PB30.11-10; qPCRBIO) according to manufacturer's protocol. PCR was performed in triplicate using a StepOnePlus system (Applied Biosystems). The expression of each target gene was normalized to the expression of GAPDH mRNA using the 2^{-ΔΔct method, presenting the differences (Δ) in threshold cycle (Ct) between the target gene and GAPDH gene. ΔCt=Ct target gene—Ct GAPDH. ΔΔCt=ΔCt treatment-ΔCt control. Experiments were repeated three times. The primers were: for CB}2 (CNR2; Gene ID 1269) (forward) 5′-ATCATGTGGGTCCTCTCAGC-3′ (SEQ ID NO: 1) and (reverse) 5′-GATTCCGGAAAAGAGGAAGG-3′ (SEQ ID NO: 2); TRIB3 (Gene ID: 57761) (forward) 5′-GGTGCTTATCAGGTGCCAAG-3′ (SEQ ID NO: 3) and (reverse) 5′-GTTGTCAGCTCAAGGATGCC-3′ (SEQ ID NO: 4); ATF4 (Gene ID: 468) (forward) 5′-GGAAACCATGCCAGATGACC-3′ (SEQ ID NO: 5) and (reverse) 5′-ACTTTCTGGGAGATGGCCAA-3′ (SEQ ID NO: 6); CHOP (Gene ID: 1649) (forward) 5′-AGCAGAGGTCACAAGCACCT-3′ (SEQ ID NO: 7) and (reverse) 5′-CTGGGGAATGACCACTCTGT-3′ (SEQ ID NO: 8);

3D models—Hydrogels (AGFCH) included Sigma-Aldrich products and were prepared using alginate (W201502; Sigma-Aldrich, USA) 22.5 mg/mL, gelatin (G9764; Bio-Basic, USA) 45 mg/mL, fibrinogen (F38791; Sigma-Aldrich, USA) 50 mg/mL, collagen (C9791; Sigma-Aldrich, USA) 2.2 mg/mL and hyaluronic acid (O8185; Sigma-Aldrich, USA) 2 mg/mL in PBS-glycerol solvent. The gel (AGFCH) was mixed with U87 cells at a concentration of 2×10⁶ cells per 400 μL gel. The solutions were mixed gently and transferred as 25 μL of gel solution to a 24-well plate. 3D models were cross-linked using CaCl₂ (A610050; Bio-Basic, USA) and thrombin (SRP6557; Sigma-Aldrich, USA) for 5 minutes, following the 3D structure was washed with PBS and then immersed in 1 mL of EMEM complete medium. Cells in the 3D structures were allowed to grow for 2 days and then the indicated treatments were administered for 8 days (treatments were repeated every 2 days). Structures were stained using EasyProbe Hoechst, as described above. To assess cell viability, the 3D structure was digested using 200 μL of 0.05M sodium citrate (C3434; Sigma-Aldrich, USA) and 0.05M EDTA (03-052-1A; Biological Industries, Israel) solution for 5 minutes. The solution was centrifuged for 4 minutes at 1700 rpm, the cell pellet was washed with PBS, and the Alamar Blue (resazurin, AR002; R&D Systems) assay was performed. Groups of more than 20 cells were identified as a colony [33]. Percentage of colony in the different treatments was calculated out of average number of colonies in the control.

Flash liquid chromatography—An Ecoflex C-18 80 g (Flash Pure, Buchi, C-18, 80 μm spherical, max. pressure 180 psi) column was used for separation, with gradient of methanol and water as the mobile phase, starting with 75% up to 100% methanol at a flow rate of 60 ml/min. Detection wavelengths were set at 220 and 280 nm and UV scan monitored from 200-400 nm. Based on the signal intensities of these two wavelengths, the system automatically collected fractions in fraction collector vials. Eluted fractions vials were divided into different fractions based on the identified peaks.

Statistical analysis—Results are presented as mean+SE of replicate analyses and are either representative of or include, at least two independent experiments. Means of replicates were subjected to statistical analysis by Tukey-Kramer test using the JMP statistical package (www(dot)jmp(dot)com/en_us/horne(dot)html, SAS Inc, NC, USA) and considered significant when P≤0.05.

### Example 1

### C. sativa Extracts and Chromatography Fractionated Fractions have a Cytotoxic Activity Against Glioblastoma Cells

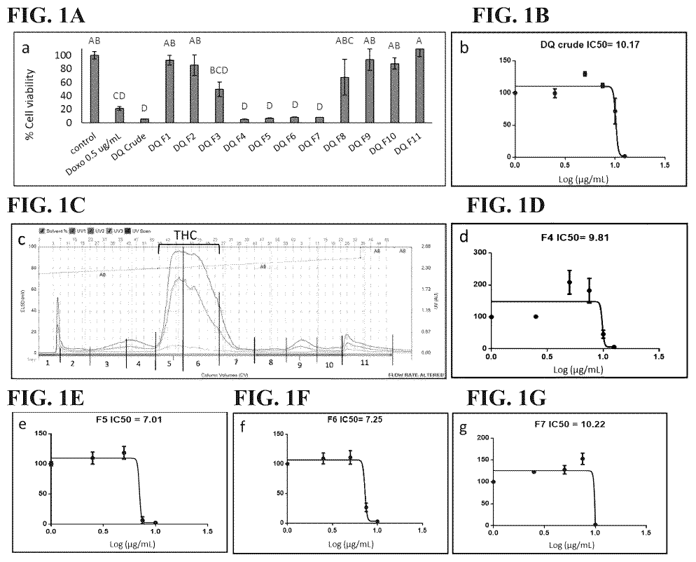

The methanol extract of high-THC C. sativa strain DQ was found to be cytotoxic to the glioblastoma multiforme (GBM) cell line A172 (FIG. 1A), with a calculated IC50 of 10.17 μg/mL following treatment for 48 hours (FIG. 1B). To identify the active compounds of the DQ extract, fractionation was performed using flash chromatography (FIG. 1C) and the cytotoxic activity of the obtained fractions on the A172 cell line was examined. Four of the 11 fractions (F4-F7) showed significant cytotoxic activity at the examined concentration, resulting in ˜90% cell death (FIG. 1A). Treatments with fraction F3 or F8 showed moderate cytotoxic activity (50% and 30% cell death, respectively; FIG. 1A). Fractions F1, F2, F9 and F10 exhibited only minor cytotoxic activity, resulting in approximately ˜10% cell death whereas treatment with F11 did not lead to cell death. Rather, it caused minor (˜110%) cell proliferation (FIG. 1A).

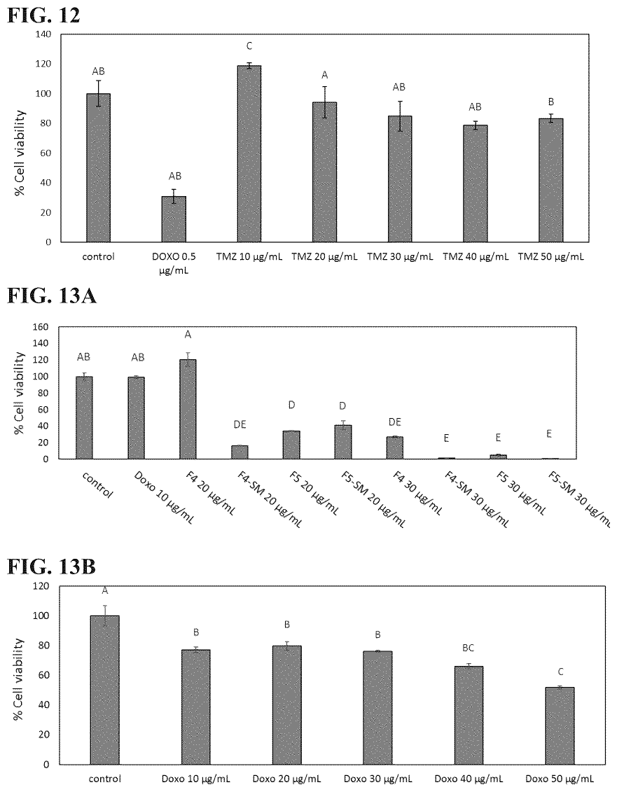

The calculated IC50 of F4, fractionated prior to the emergence of the THC peak from flash chromatography (FIG. 1C) was 9.81 μg/mL (FIG. 1D). Among the fractions allocated at the THC peak, designated F5-F7 (FIG. 1C), F5 was the most active with IC50 of 7.01 μg/mL (FIG. 1E). IC50 values of F6 and F7 were 7.25 and 10.22 μg/mL, respectively (FIGS. 1F-G). Notably, at sub-lethal concentrations, the crude extract and some of the fractions, especially F4 and F7, led to cell proliferation (˜200% and ˜150%, respectively; FIGS. 1B, 1D and 1G respectively). TMZ did not lead to substantial A172 cell death even at relatively high concentrations (i.e., up to 50 μg/mL; FIG. 12).

F4 and F5 were also active on the U87 cell line, however at higher concentrations in comparison to their activity on the A172 cell line (FIG. 13A). Doxorubicin at relatively high concentrations (i.e., up to 50 μg/mL) was only moderately active on this cell line under the examined conditions (FIG. 13B).

### Example 2

### The Chemical Composition of C. sativa Extracts and Chromatography Fractionated Fractions

The composition of the crude extract and active fractions, i.e., F4-F7, was chemically characterized using HPLC (Table 1 hereinbelow). Phytocannabinoid content in the crude extract was 55.2% while terpenes constituted ˜32% of the crude extract. Phytocannabinoid content in fractions F4-F7 was ˜60-65% on average. F5 and F6 were similar in content and included mainly THC (Table 1 hereinbelow), F7 included mainly THC and cannabichromene (CBC) and F4 contained mainly cannabigerol (CBG) (Table 1 hereinbelow). Table 2 hereinbelow shows terpenes content in F4-F7, as determined by GC/MS. In addition, the chemical composition of two additional C. sativa extracts, namely sCBD and PARIS was determined (Table 7 hereinbelow).

TABLE 1
Phytocannabinoid percentage of total phytocannabinoids in the crude
DQ strain extract, and the F4-F7 fractionated from the extract.

| | compound | | | | | | | | | |
|---|---|---|---|---|---|---|---|---|---|---|
| Fraction | CBC | CBD | CBDV | CBDVA | CBG | CBGA | CBN | THC | THCA | THCV |
| Crude | 5.0 | 0.1 | <0.1 | <0.1 | 4.0 | 0.1 | 1.8 | 87.4 | 0.5 | 1.0 |
| F4 | — | 5.8 | — | — | 80.3 | — | 2.8 | — | — | 11 |
| F5 | — | — | — | — | 3.7 | — | 4.6 | 91.7 | — | — |
| F6 | — | — | — | — | 2.2 | — | 0.9 | 96.9 | — | — |
| F7 | 23.5 | — | 1.8 | 0.9 | 1.3 | — | 1.9 | 68.9 | 1.7 | — |

TABLE 2
Percentage of terpenes out of total terpenes
in F4-F7 fractions of the DQ extract.

| Terpenes (%) | F4 | F5 | F6 | F7 |
|---|---|---|---|---|
| α-Bisabolol | 1.38 | 0.65 | | |
| α-Eudesmol | 62.35 | 19.29 | | |
| γ-Eudesmol | 18.13 | 23.78 | | |
| 9-Isopropyl-1-methyl-2-methylene- | | 0.74 | | |
| 5-oxatricyclo[5.4.0.0(3,8)]undecane | | | | |
| Agarospirol | 1.80 | | | |
| Azulemanthanol | 5.43 | | | |
| Bulnesol | | 15.46 | | |
| Caryophyllene oxide | | 1.57 | | |
| α-Cedrene | | 0.53 | | |
| β-Selinene | | 2.32 | | 5.19 |
| Guaiol | 10.92 | 26.90 | | |
| Nerolidol | | 6.33 | | |
| Selina-3,7(11)-diene | | 2.43 | | 7.78 |
| Caryophyllene | | | 0.24 | 61.54 |
| α.-Bergamotene | | | 86.00 | 3.56 |
| Muurola-3,5-diene | | | 1.47 | |
| β-Curcumene | | | 0.49 | |
| Thujopsene | | | 0.25 | |
| Valencene | | | 1.23 | |
| Longifolene | | | 5.73 | |
| δ-Selinene | | | 1.78 | |
| Patchoulene | | | 1.09 | |
| D-Limonene | | | | 0.36 |
| (−)-Guaia-6,9-diene | | | | 0.58 |
| γ-Gurjunene | | | | 0.45 |
| γ-Muurolene | | | | 1.86 |
| Naphthalene | | | | 2.20 |
| α-Selinene | | | | 1.83 |
| Bicyclogermacrene | | | | 2.22 |
| Cadina-1(10),4-diene | | | | 0.50 |
| α-Guaiene | | | | 1.13 |
| β-Guaiene | | | | 5.98 |
| Alloaromadendrene | | | | 1.26 |
| β-Maaliene | | | | 1.82 |

TABLE 7
Phytocannabinoid percentage of total phytocannabinoids
in the crude sCBD and PARIS strains extracts.

| | | % from |
|---|---|---|
| | Phytocannabinoid | phytocannabinoid |
| sCBD | CBD | 88.7 |
| crude | THC | 1.77 |
| extract | CBG | 3.49 |
| | CBC | 4.64 |
| | CBDV | 1.02 |
| | CBDA | 0.34 |
| PARIS | CBC | 2.68 |
| crude | CBD | 58.82 |
| extract | CBG | 3.37 |
| | THC | 35.1 |

### Example 3

### Combinations of Pure Cannabinoids Mimicking Chromatography-Fractionated Fractions F4 and F5 have Cytotoxic Activity Against Glioblastoma Cells

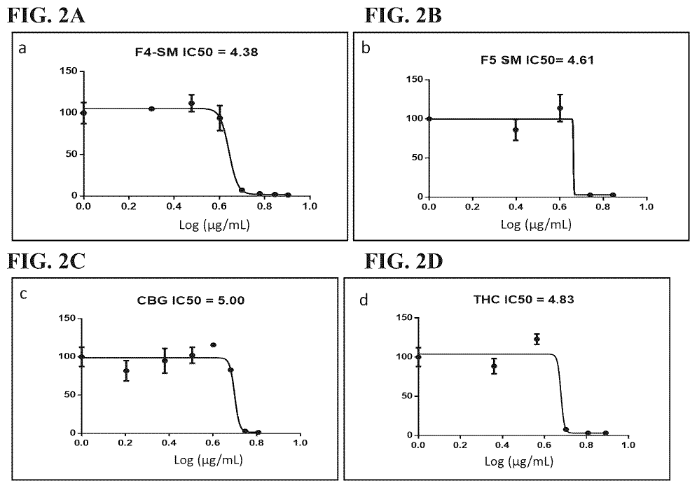

In order to confirm the active compounds of F4 and F5, IC50 values of phytocannabinoid standard mix (SM) of each fraction were examined and calculated. SM is the mix of phytocannabinoid standards equivalent of the primary phytocannabinoids in Table 1 hereinabove, at the appropriate ratios to be as close to F4 and F5 as possible. F4-SM had an IC50 of 4.38 μg/mL (FIG. 2A), which was lower than that of F4 (9.81 μg/mL). F5-SM showed IC50 value of 4.61 μg/mL (FIG. 2B), again lower than F5 (7.01 μg/mL). F4-SM showed significant reduction of cell proliferation at sub-lethal concentrations (˜110% of cell proliferation in comparison to −200% in F4 treatments; FIG. 2A). F5-SM led to similar levels of cell proliferation at sub-lethal concentrations as F5 (˜115% and ˜110%, respectively; FIG. 2B). Interestingly, CBG, the primary molecule of F4, was less active than F4-SM (IC50 of 4.38 and 5.00 μg/mL for F4-SM and CBG, respectively; FIG. 2C). Similarly, THC, the primary molecule of F5, was less active than F5-SM (IC50 of 4.61 and 4.83 μg/mL for F5-SM and THC, respectively; FIG. 2D).

F4-SM and F5-SM were also active on the U87 cell line, however at higher concentrations in comparison to the active concentrations on A172 cells (FIG. 13A). F4-SM activity was higher than F4 on U87 cells (FIG. 13A).

### Example 4

### The Involvement of the Cannabinoid Receptors in the Cytotoxic Activity Against GBM

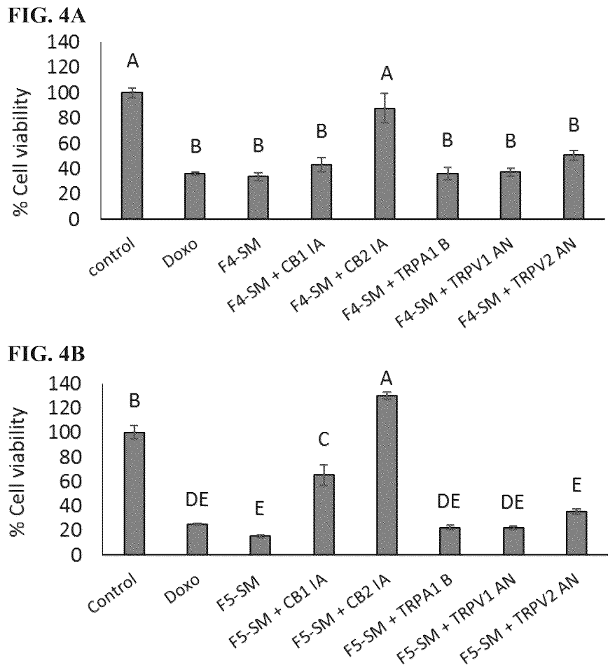

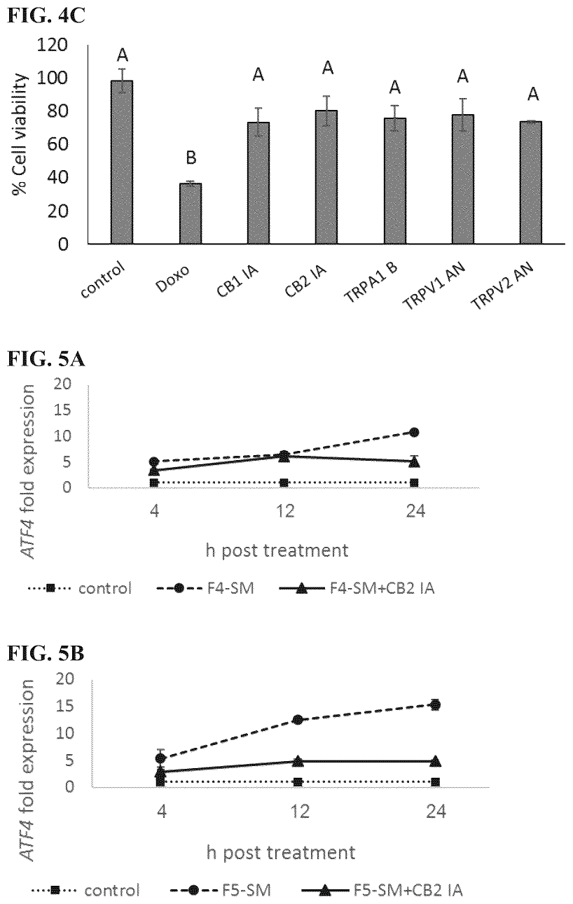

Following, the effect of adding CB1 or CB2 invers agonist (IA), TRPV1 or TRPV2 antagonists (AN) or TRPA1 blocker (B) on F4-SM and F5-SM activity was determined. A172 cells were treated with F4-SM and F5-SM with or without the IA, AN or B. In the presence of CB2, the cytotoxic effect of F4-SM was significantly reduced (87.9% vs. 33.8% viable cells with or without CB2 IA, respectively; FIG. 4A). In the case of F5-SM, addition of CB2 IA led to cell proliferation (129.9% vs. 15.3% viable cells with or without CB2 IA, respectively; FIG. 4B). Addition of CB1 IA to F5-SM treatment led to inhibition of the cytotoxic activity, but to a lesser extent than CB2 IA (65.1% vs. 15.3% cell viability for F5-SM with or without CB1 IA, respectively; FIG. 4B).

Treatment with F4-SM in the presence of TRPV1 or TRPV2 AN, TRPA1 B or CB1 IA, or F5-SM treatment with TRPV1, TRPV2 AN and TRPA1 B did not significantly affect the cytotoxicity of the treatments (FIGS. 4A-B). CB1 and CB2 IA, TRPV1 and TRPV2 AN and TRPA1 B reduced A172 cell viability to a minor, non-significant extent (FIG. 4C).

CB2 (CNR2) was expressed in A172 cells and its expression was reduced with the F5-SM treatments and induced with F4-SM treatment (Table 3 hereinbelow). However, no CB1 expression was detected in these cells.

Taken together, CB2 IA significantly blocked F4-SM and F5-SM cytotoxic activity. Hence, the CB2 receptor might be involved to some extent in F4-SM and F5-SM activity. Further CB1 IA blocked activity of F5-SM, however CB1 gene expression could not be detected in the A172 cells. Since the CB1 IA used (AM251) sometimes also acts as an IA of the CB2 receptor (e.g., EC50 value of 650±30 nM; [46]), it might be that the reduction in F5-SM activity in the presence of AM251 was a result of its IA activity on the CB2 receptor. In addition, as neither the TRPA1 blocker, nor TRPV1 or TRPV2 antagonists reduced F4-SM or F5-SM activity, TRPA1, TRPV1 and TRPV2 are probably not involved in the cytotoxic activity of these compositions on GBM cells.

TABLE 3
Quantitative PCR determination of the RNA steady state level
of CB2 receptor (CNR2) gene in A172 cell line following treatment
with F4-SM or F5-SM (10 μg/mL) for 12 hours relative to
control. Methanol (control) treatment served as a solvent
(vehicle) control. Gene transcript values were determined
by quantitative PCR. Mean values ± SE are shown (n = 3).

| | CB2 receptor (CNR2) |
|---|---|
| Treatment | relative expression |
| F4-SM | 2.32 ± 0.49 |
| F5-SM | 0.47 ± 0.09 |

### Example 5

### Chromatography-Fractionated Fractions F4 and F5 and Combinations of Pure Cannabinoids Mimicking Fractions F4 and F5 Induce GBM Cell Cycle Arrest, ER-Stress and Apoptosis, Reduce Cell Migration and Disintegrate F-Actin

The effects of treatment with F4, F4-SM, F5 or F5-SM on cell apoptosis, cell cycle arrest, ER stress, migration and cytoskeleton was further determined.

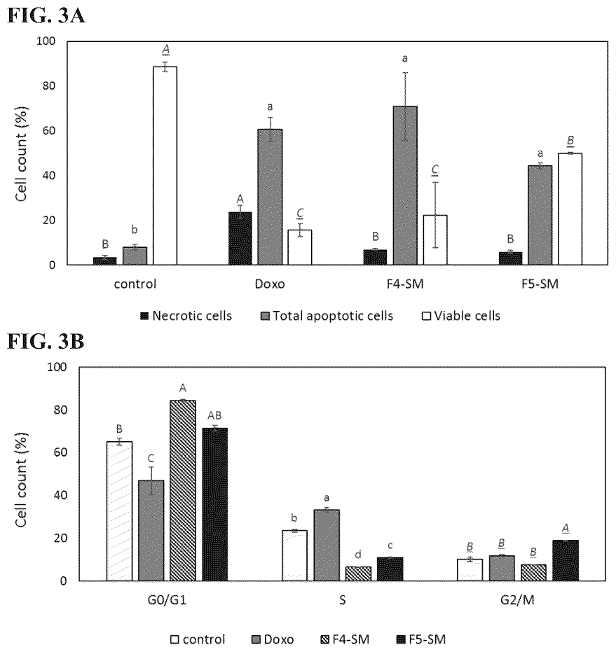

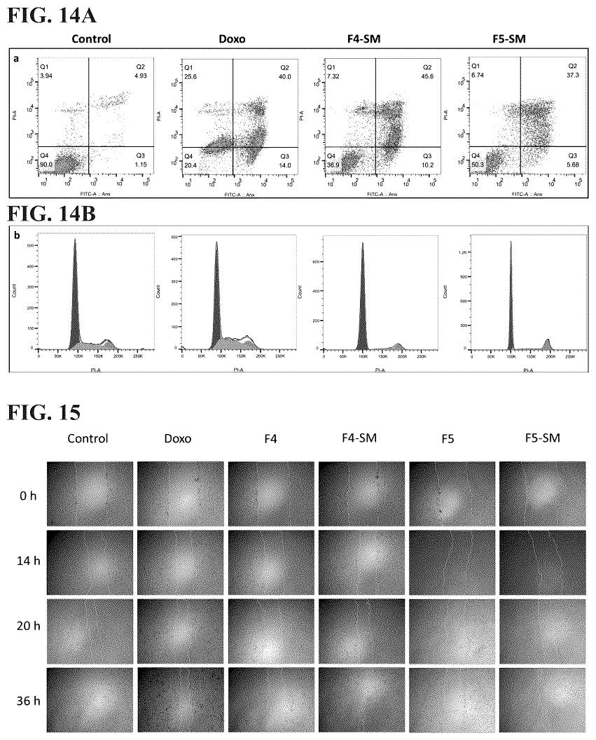

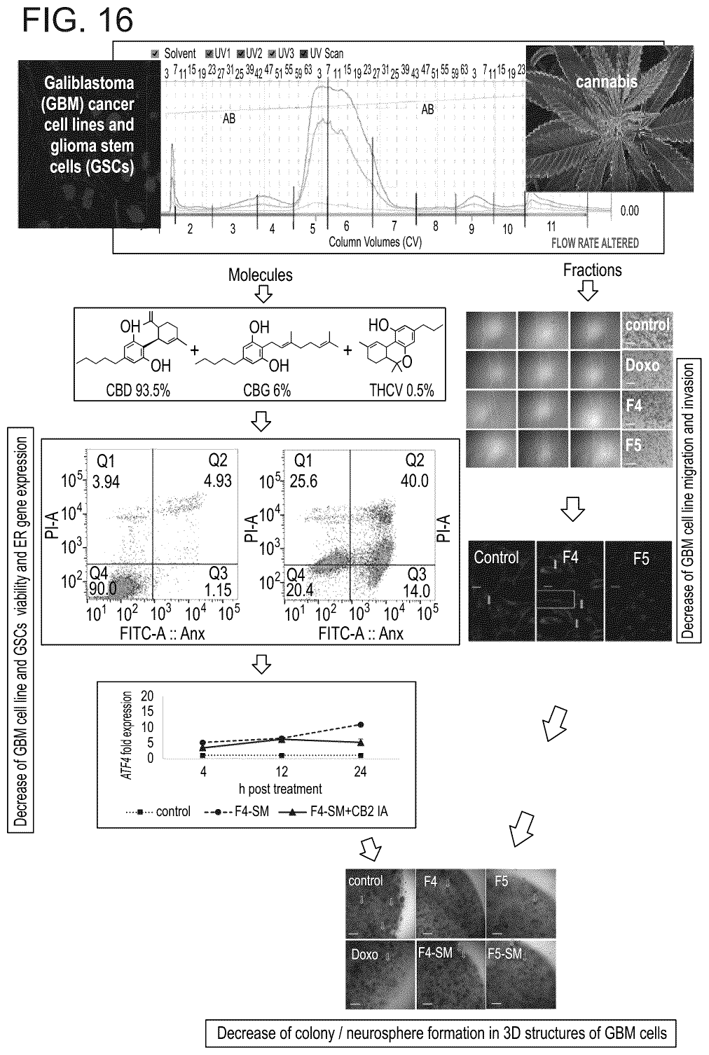

Treatment with F4-SM or F5-SM for 48 hours led to 70.8% and 44.3% cell apoptosis, respectively, in comparison to 8.0% apoptosis in the vehicle control and 60.6% in doxorubicin positive control (FIGS. 3A and 14A). Moreover, treatments of A172 with F4-SM and F5-SM led to a lower percentage of necrotic cells (6.9% and 5.8% respectively) compared to doxorubicin (23.8%) but higher than in the methanol control (3.4%; FIGS. 3A and 14A).

Treatment of A172 for 24 hours with F4-SM led to an increase in the percentage of cells in the G1 phase of the cell cycle (84.5%) in comparison to the control (vehicle) treatment (65.2%; FIGS. 3B and 14B). F5-SM treatment led to a significant enrichment in the percentage of cells in the G2-M phase (18.8%) in comparison to 10.2% in the control, 11.6% in the doxorubicin and 7.6% in F4-SM (FIGS. 3B and 14B). Both F4-SM and F5-SM treatments led to a reduction in the percentage of S phase cells (6.5% and 11.0%, respectively) in comparison to the vehicle control (23.5%; FIGS. 3B and 14B).

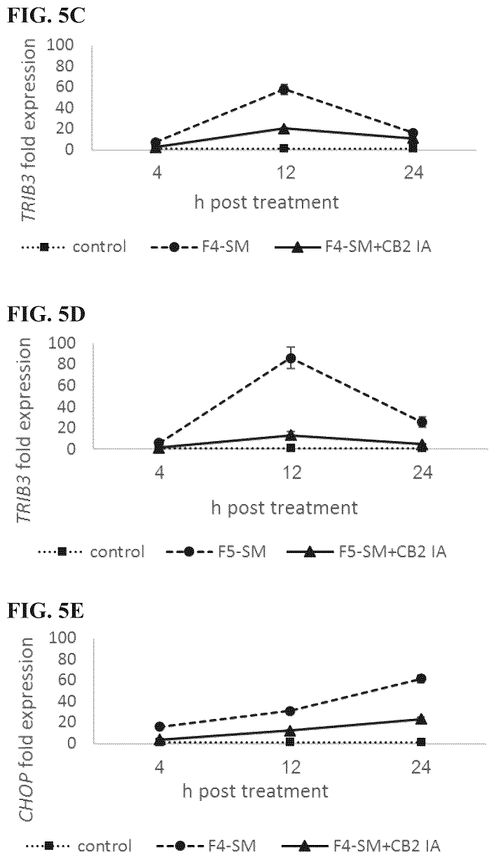

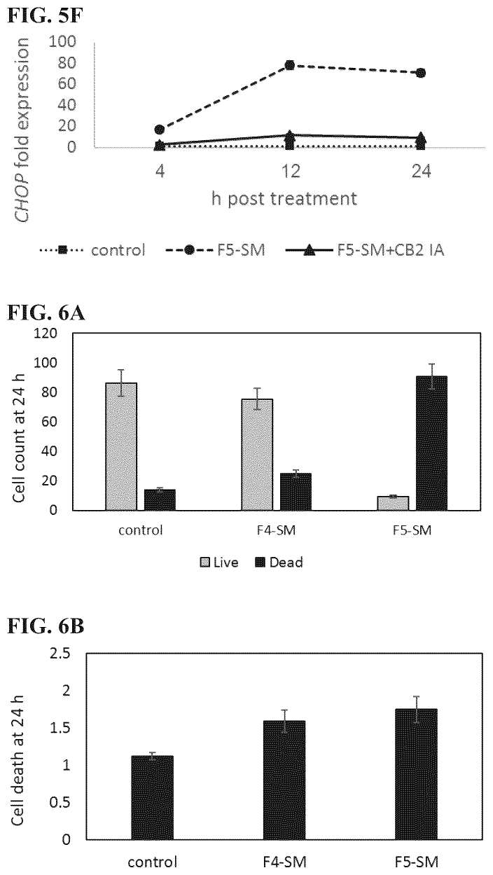

To explore the possible induction of ER-stress by F4-SM and F5-SM treatments, the expression of ATF4, TRIB3 and CHOP (DDIT3-3) genes was determined. F4-SM and F5-SM treatments substantially induced expression of these gene (FIGS. 5A-F and Table 4 hereinbelow), with the highest ATF4 expression at 24 hours (FIGS. 5A-B and Table 4 hereinbelow). TRIB3 expression was the highest at 12 hours in both treatments (FIGS. 5C-D and Table 4 hereinbelow). CHOP expression was the highest at 24 hours with F4-SM or 12 and 24 hours with F5-SM treatments (FIGS. 5E-F and Table 4 hereinbelow). Co-treatment with CB2 IA considerably reduced induction by F4-SM or F5-SM of all gene expression (FIGS. 5A-F and Table 4 hereinbelow).

TABLE 4
Statistical analysis for quantitative PCR determination of the
RNA steady state level in A172 cell line of ATF4, TRIB3 and
CHOP (DDIT3-3) genes following treatment with F4-SM or F5-SM
relative to control, presented in FIGS. 5A-F. Letters with
similar style (uppercase, lowercase, italic and/or bold letters)
were compared for each gene and time post treatment.

| | Treatment/time post | | | |
|---|---|---|---|---|
| | treatment | 4 h | 12 h | 24 h |
| ATF4 | Control (for F4-SM) | C | B | C |
| | F4-SM | A | A | A |
| | F4-SM + CB2 IA | B | A | B |
| | Control (for F5-SM) | b | c | c |
| | F5-SM | a | a | a |
| | F5-SM + CB2 IA | ab | b | b |
| TRIB3 | Control (for F4-SM) | B | C | C |
| | F4-SM | A | A | A |
| | F4-SM + CB2 IA | B | B | B |
| | Control (for F5-SM) | a | b | b |
| | F5-SM | a | a | a |
| | F5-SM + CB2 IA | a | b | b |
| CHOP | Control (for F4-SM) | C | C | C |
| | F4-SM | A | A | A |
| | F4-SM + CB2 IA | B | B | B |
| | Control (for F4-SM) | b | b | c |
| | F5-SM | a | a | a |
| | F5-SM + CB2 IA | b | b | b |

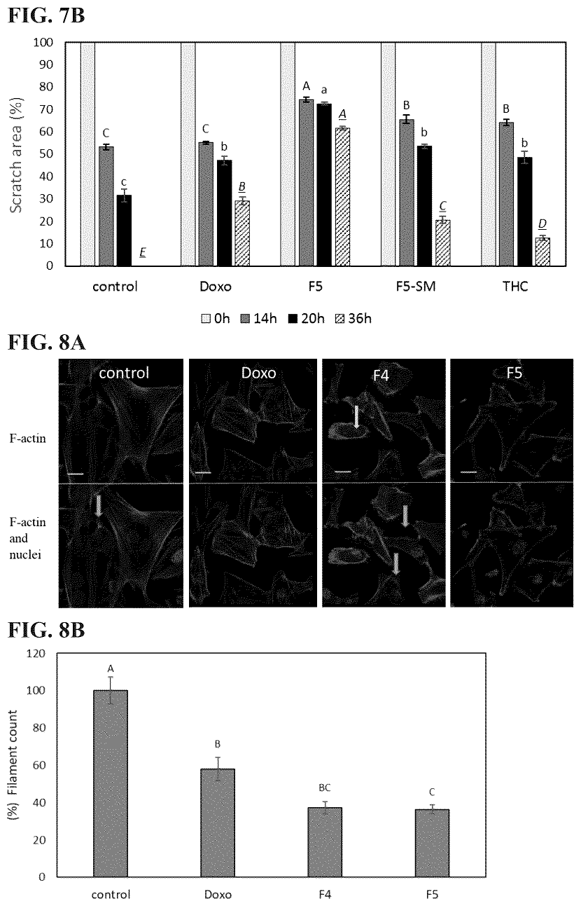

To examine the ability of the fractions and SM to attenuate cancer cell motility, the effects of F4, F5 and their corresponding SM on cell migration at sub-lethal concentrations were examined using scratch assays. 36 hours treatment of the A172 cell line with F4 or F5 led to a significant inhibition of cell migration (37.7% and 61.6%, respectively), in comparison to the vehicle control (FIGS. 7A-B and 15). Treatment with F4-SM or F5-SM at concentrations corresponding to those in the extract fractions led to less inhibition of cell migration, 9.5% and 20.5%, respectively in comparison to the control (FIGS. 7A-B and 15). Treatment with the primary molecules of F4 or F5, i.e., CBG or THC, led to even less inhibition of cell migration (2.8 and 12.5% respectively in comparison to the control). Doxorubicin treatment also inhibited cell migration (28.8% in comparison to control), but this was lower than F4 or F5 (FIGS. 7A-B and 15).

Following, changes in cytoskeleton structures in A172 were evaluated following treatment with F4 or F5. F-actin filaments of cells treated with vehicle control formed organized and distinct networks with few protruding filopodia (FIG. 8A). However, the F-actin network disappeared in cells treated with F4, and was replaced with diffused dot-like actin structures (FIG. 8A yellow arrows and FIG. 8B). Furthermore, F4 treatment led to the induction of F-actin filopodia compared to the control (FIG. 8A, green arrows). The F-actin network also disappeared in cells treated with F5 (FIGS. 8A-B). However, F5 treatment reduced the appearance of filopodia in cells in comparison to F4 (FIG. 8A). Doxorubicin effect on F-actin structures was less pronounced in comparison to that of F4 or F5 (FIGS. 8A-B).

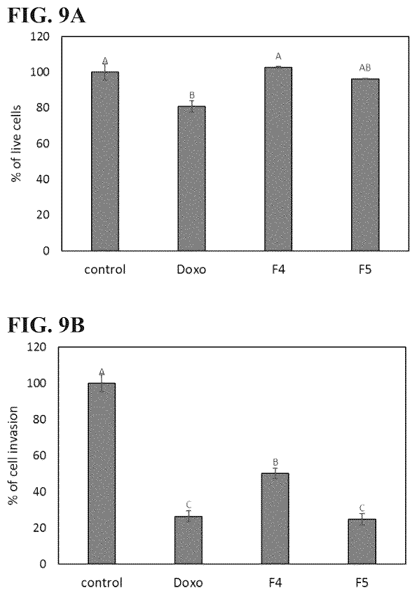

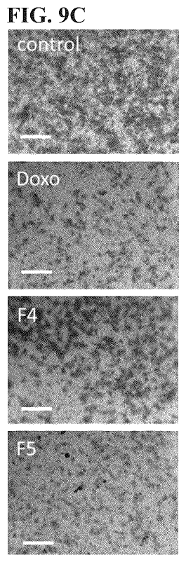

In addition, the effect of treatment with FF4 or F5 on cell invasion was determined using the trasnwell assay. At mostly sub-lethal concentrations (FIG. 9A), F4 and F5 treatments substantially reduced cell invasion in a transwell assay, F5 to a greater extent (43.6 vs 18.6%) relative to a vehicle control (FIG. 9B). As a positive control, doxorubicin treatment reduced cell invasion by 22.5% in comparison to the vehicle control (FIG. 9B), in agreement with [34]. FIG. 9C shows examples of cells that invaded the membrane at 24 hours for each treatment.

### Example 6

### Chromatography-Fractionated Fractions F4 and F5 and Combinations of Pure Cannabinoids Mimicking Fractions F4 and F5 Reduce Gbm Colony Formation

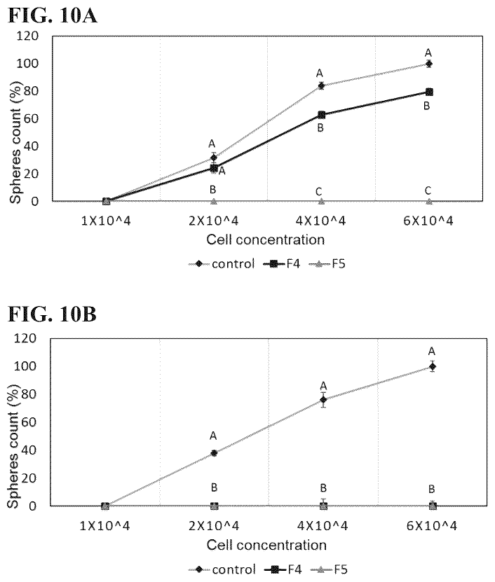

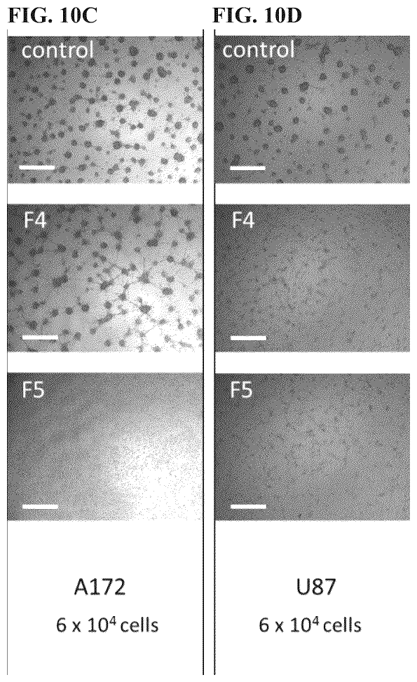

To examine the effect of F4, F5 on colony formation, cells were sorted following treatments to live cells and these live cells were re-seeded and allowed to form colonies. Treatment of A172 cells with F4 reduced colony formation to a small but significant extent (FIGS. 10A and 10C). However, treatment with F5 completely abolished colony formation of A172 cells (FIGS. 10A and 10C). In U87 cell line, F4 and F5 treatments led to complete abolishment of colony formation, at all examined cell concentrations (FIGS. 10B and 10D).

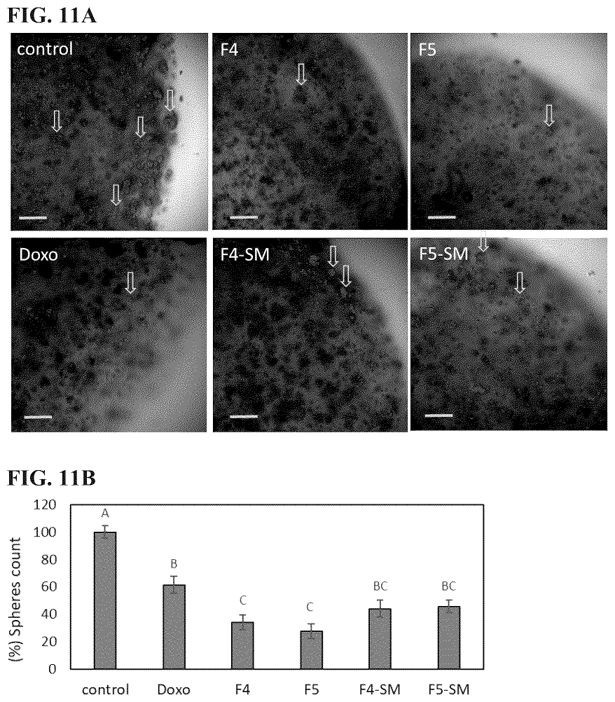

To examine the effect of the fractions on colony formation in 3D structures, U87 cells were seeded in extra cellular matrixes, forming 3D droplets, and were treated with F4, F5, F4-SM, F5-SM or doxorubicin. Multiple colonies were formed in the control (˜20; FIGS. 11A-B). However, in the F4 and especially in F5 treated structures, cells were dispersed and less colonies were formed (FIGS. 11A-B). The number of live cells in these structures was also substantially reduced (Table 5 hereinbelow). Treatments with F4-SM or F5-SM at concentrations corresponding to those in the fractions reduced colony formation, similarly to doxorubicin, but to a somewhat lesser extent than the fraction treatments (FIGS. 11A-B).

TABLE 5
Cell viability of U87 cells from 3D structures following 48
hours treatment with F4, F5, F4-SM or F5-SM at the indicated
concentrations. Cell viability was determined by 3D structure
disintegration and cell dispersal following Alamar Blue (resazurin)
assay. Control - vehicle control (3% v/v methanol). Means
(n = 3) with different letters are significantly different
from all combinations of pairs according to the Tukey-Kramer
honest significant difference (HSD; P ≤ 0.05).

| Treatment | Mean (% of Live cells) |
|---|---|
| control | 98.52 ± 0.08^{a} |
| Doxorubicin 2 μg/ml | 37.22 ± 3.08^{c} |
| F4 20 μg/ml | 11.98 ± 0.44^{d} |
| F4-SM 12.5 μg/ml | 17.39 ± 1.14^{a} |
| F5 16.5 μg/ml | 12.32 ± 0.02^{d} |
| F5-SM 10 μg/ml | 72.81 ± 0.05 ^{b} |

### Example 7

### Chromatography-Fractionated Fractions F4 and F5 and Combinations of Pure Cannabinoids Mimicking Fractions F4 and F5 have a Cytotoxic Effect Against Glioblastoma Stem Cells

GBM has a dismal prognosis that is partly attributed to the presence of GSCs that exhibit self-renewal abilities and resistance to radiation and chemotherapy and are implicated in tumor infiltration and recurrence. Indeed, one of the barriers to successful treatment of GBM is the eradication of the GSC subpopulation. Although GSCs represent only a small percentage of the tumor cells in GBM, they are implicated in tumor recurrence. Therefore, identifying treatment that target these cells is of great importance.

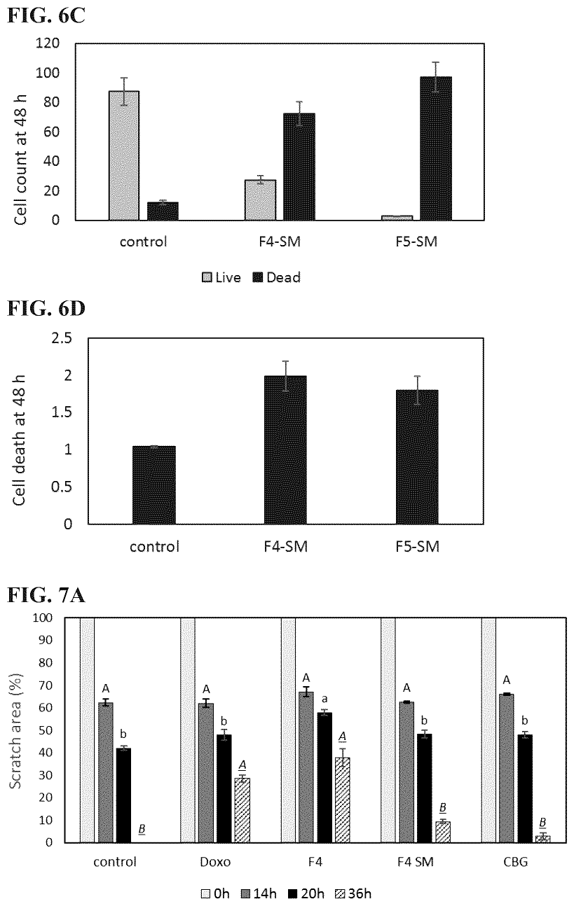

To analyze the cytotoxic effects of F4-SM and F5-SM on GSC neurosphere cultures (GSC-1) that were generated from a GBM primary tumor were employed. The GSCs were maintained as spheroids and their self-renewal, differentiation and tumorigenic abilities were validated as previously reported [30-31]. Treatment of GSC-1 with F4-SM and F5-SM at a concentration of 10 μg/mL induced a strong cytotoxic effects that was already observed following 24 hours of treatment (FIGS. 6A-B). Treatment of GSCs with F4-SM for 48 hours further increased cell death (FIGS. 6C-D). F5 exerted a stronger cytotoxic effect already 24 hours following treatment and at 48 hours following treatment, the majority of the treated cells exhibited cell death (FIGS. 6A-D).

### Example 8

### Determination of Cannabis Strain with Cytotoxic Activity Against Ovarian Cell Lines

### Materials and Methods

### Extracts Preparation

Fresh or dry specimens of dry C. sativa inflorescence strains were frozen at −20° C. using liquid nitrogen. Frozen inflorescences were ground by mortar and pestle and placed in 15 mL tubes. Absolute ethanol was added to each inflorescence powder sample at a sample-to-absolute ethanol ratio of 1:4 (w/v). The samples were mixed thoroughly on a shaker for 30 min, and then the extract was filtered through a filter (0.2 PVDF syringe filter) by syringe filtration. The filtrate was transferred to new tubes. The solvent was evaporated under nitrogen. The dried extract was weighed, and then resuspended in absolute methanol (volume of solvent added according to the desired concentration) and filtered through a 0.45 μm syringe filter. For the treatments, the resuspended extract was diluted according to cell cultures. Decarboxylation was carried out by heating the dry extract to 220° C. for 10 min. The heated extract was dissolved in methanol and filtered through a 0.45 μm syringe filter. Following evaporation, the weighted extract was re-suspended in methanol to the desired concentration.

### Extract Fractionation

A flash chromatography apparatus (Flash Pure, Buchi, C-810) equipped with a diode array detector was used to fractionate the decarboxylated crude extract. An Ecoflex C-18 80 g, 50 μm spherical, max. pressure 180 psi column was used for separation, with 80-85% methanol in water as the mobile phase. The flow rate was 30 mL/min. The organic solvent (methanol) of each fraction was separately removed by using a rotary vacuum evaporator at 30° C. The remaining aqueous phase containing the compound of interest was lyophilized to obtain a dried powder. Each dried fraction tube was weighed separately and reconstituted by methanol to produce the required concentrations, and stored at −20° C.

### Results

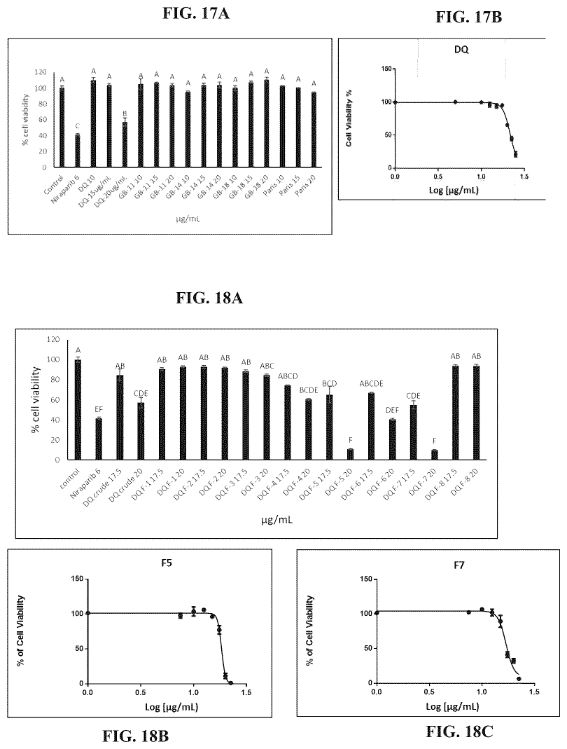

Extracts of several C. sativa strains were examined for cytotoxic activity against ovarian cancer cell line HTB75. DQ (IMC, Israel), a high Δ9-tetrahydrocannabinol (THC) strain, was the most effective with the highest examined concentration (20 μg/mL) resulting in ˜90% cell death (FIG. 17A). Other examined strains included GB-11 (THC:CBD:CBG 22:40:30; Israel GenenBank, IGB), GB-14 (THC:CBD:CBG 55:9:28; IGB) and GB-18 (High THC; IGB) and a THC:CBD (22:35) strain (Paris, IMC) were not cytotoxic to the cells, at the tested concentrations (FIG. 17A). IC50 for the crude extract was determined to be 21.51 μg/mL (FIG. 17B).

### Example 9

### Identification of Active Fractions of Dq Cannabis Strain

The most active extract, i.e. DQ, was fractionated as described above. Following, activity of fractions was examined on HTB75 cells. Four of the 11 fractions (F4, F5, F6 and F7) showed significant cytotoxic activity at the examined concentrations. F5 and F7 were the most active with the highest examined concentration (20 μg/mL) resulting in ˜90% cell death (FIG. 18A). F6 and F4 were less active with the highest examined concentration (20 μg/mL) resulting in ˜60% and ˜40% cell death, respectively (FIG. 18A). Treatments with F1-F3 and F8 showed no cytotoxic activity against HTB75 cells (FIG. 18A). IC50 of the most active fraction was determined to be 18.36 and 16.95 μg/mL for F5 and F7, respectively (FIGS. 18B-C). F5 and F7 phytocannabinoids composition is as described in Table 1 above, and also in Peeri et al., 2021. Terpenes composition of F5 as in Peeri et al., 2021.

TABLE 6

| Compound | % of total in F7 |
|---|---|
| D-Limonene | 0.37 |
| α Bergamotene | 3.62 |
| Caryophyllene | 62.64 |
| (−)-Guaia-6,9-diene | 0.59 |
| γ Gurjunene | 0.46 |
| γ Muurolene | 1.90 |
| onaphthalene | 2.24 |
| α selinene | 1.86 |
| β selinene | 5.29 |
| Bicyclogermacrene | 2.26 |
| Cadina-1(10),4-diene | 0.51 |
| α Guaiene | 1.15 |
| β Guaiene | 6.08 |
| Alloaromadendrene | 1.28 |
| Selina-3,7(11)-diene | 7.92 |
| β maaliene | 1.85 |

### Example 10

### Determination of Activity of the Phytocannabinoid Standard Mixes of F5 and F7

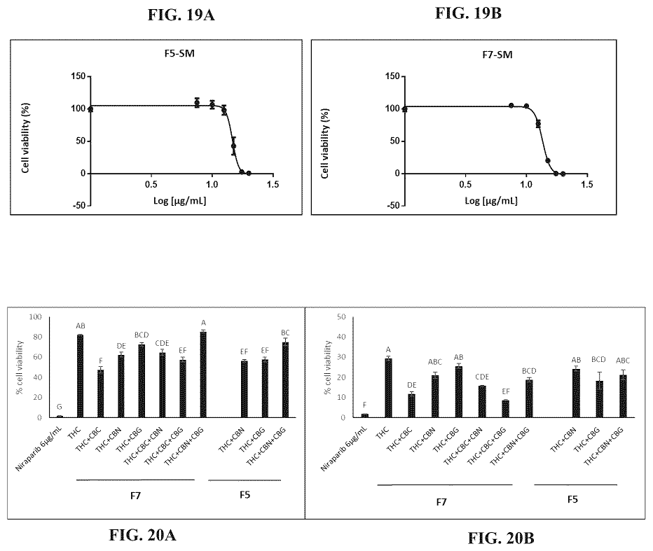

In order to confirm the active compositions of F5 and F7, standard mixes (SM) were prepared as described above. F5-SM had an IC50 of 14.67 μg/mL (FIG. 19A), which was lower than that of F5 (18.36 μg/mL; FIG. 18B). F7-SM showed IC50 value of 13.56 μg/mL (FIG. 19B), again lower than that of F7 (16.95 μg/mL; FIG. 18C). Activity of F5, F7 and their SM on cell viability of another ovarian cell line, HTB161, was examined. F5 was less active to some extent on this cell line in comparison to HTB75 (IC50 of 26.19 and 18.36 μg/mL for HTB161 and HTB75, respectively; FIGS. 18A-C). F7 was more active on this cell line in comparison to HTB75 (IC50 of 15.67 and 16.95 μg/mL for HTB161 and HTB75, respectively. F5-SM was more active and F7-SM less active than the plant fractions on this cell line (IC50 of 25.11 and 25.09 μg/mL for F5-SM and F7-SM respectively; not shown).

### Example 11

### Determination of the Most Effective Combination of Phytocannabinoids

To determine which are the most effective main phytocannabinoid combinations, combinations of THC, CBG, CBC and CBN were examined in ratios found in F5 or F7, at fixed total concentration in comparison to THC (FIGS. 20A-B). At concentration of 13 and 15 μg/mL (FIG. 20A and FIG. 20B, respectively), THC only was less effective than various combinations of THC, CBC, CBG and CBN in ratios based on those found in F5 or F7 (FIG. 20A-B). The most effective combinations for both 13 and 15 μg/mL treatments were those of THC+CBC and THC+CBC+CBG in ratios based on those found in F7 (ratios of 7.5:2.5 and 7.4:2.5:0.1, respectively; FIGS. 20A-B). For F5 combinations, THC+CBG was the effective but not significantly different from the other F5 combinations (FIGS. 20A-B).

### Example 12

### Determination of the Effect of F5, F7, F5-SM or F7-SM Treatments on Cell Apoptosis

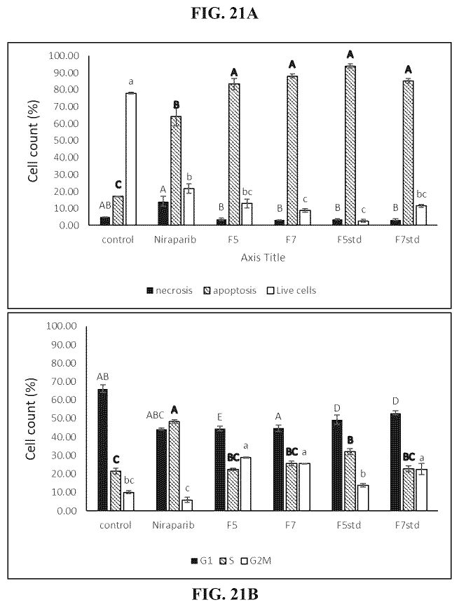

Treatment with F5 and F7 for 48 h led to 83.3% and 88.0% cell apoptosis, respectively, in comparison to 17.3% apoptosis in the vehicle control. Treatment with the phytocannabinoids mixes, F5-SM and F7-SM let to similar high levels of cell apoptosis: 93.9% and 85.0%, respectively (FIG. 21A). The chemotherapy drug niraparib led to 64.1% of apoptotic cells (FIG. 21A). Only low levels of necrosis were recorded with the cannabis treatments, similar to the control (FIG. 21A).

### Example 13

### Determination of the Effect of F5, F5-SM, F7 or F7-SM Treatments on Cell Cycle Arrest

HTB-75 treatment with F5, F7 and F7-SM for 24 h led to a significant increase in the percentage of cells in the G2/M phase of the cell cycle (28.9, 25.5 and 22.4%, respectively) in comparison to the control (vehicle) treatment (10.1%; FIG. 21B). A slight but significant increase in S phase was recorded for the F5-SM treatment (31.9%) in comparison to 21.4% in the control (FIG. 21B). Niraparib led mainly to S phase arrest (48.3%) (FIG. 21B).

### Example 14

### Determination of the Involvement of CB1 and CB2 Receptor Inverse Agonists, TRPA1 Receptor Blocker and TRPV1 and TRPV2 Receptors Antagonists on Cytotoxic Activity

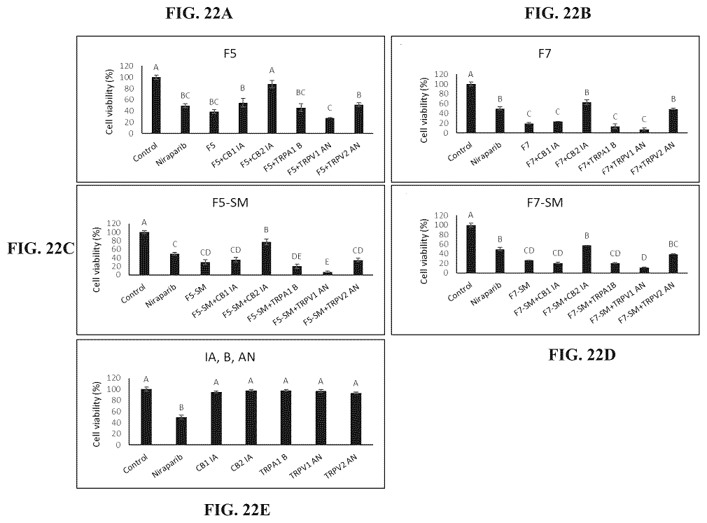

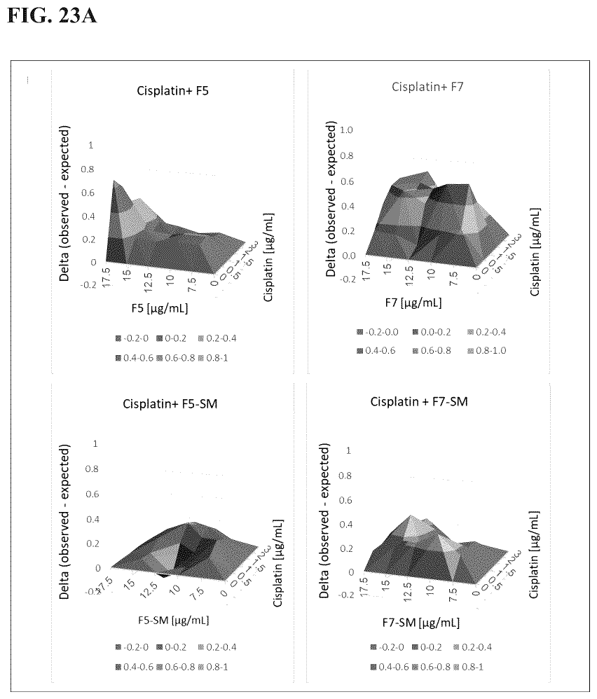

The effect of adding CB1 or CB2 inverse agonists (IA), TRPV1 or TRPV2 antagonists (AN) or TRPA1 blocker (B) on F5, F7, F5-SM and F7-SM activity was determined. HTB75 cells were treated with F5, F7, F5-SM or F7-SM with or without the IA, AN or B. In the presence of CB2 IA, the cytotoxic effect of F5, F7, F5-SM or F7-SM was significantly reduced (38.5, 19.3, 29.6, 26.3% vs. 45.8, 62.7, 77.3, 57.1% viable cells without or with CB2 IA, respectively; FIG. 22A-D). Also, TRPV2 AN co-treatment with F7 and F7-SM interfered to some extent with their activity (significantly with F7; FIGS. 22B, D). Percentage of cell viability for F7 or F7-SM were 29.6 or 26.3% vs. 48.9 or 39.2%, without or with TRPV2, respectively (FIGS. 22B, D). Treatments with F5, F5-SM, F7 or F7-SM in the presence of TRPV1 AN, TRPA1 B or CB1 IA, did not significantly affect the cytotoxicity of the treatments (FIGS. 22A-D). CB1 or CB2 IA, TRPV1 or TRPV2 AN or TRPA1 B did not affect HTB75 cell viability (FIG. 22E).

### Example 15

### Determination of the Effect of Combinations Between the Most Active Cannabis Fractions and Chemotherapy Drugs

Chemotherapy drugs concentrations were examined for combinational activity with F5, F7, F5-SM and F7-SM. Considerable synergy (in scale of 0 to 1) was obtained for combinations of cisplatin with the plant F5 (a peak of 0.4-0.6 at the higher F5 concentrations; FIG. 17A) and F7 (a peak of 0.4-0.6 at all examined F7 concentrations; FIG. 17A). However, F5-SM and F7-SM did not show considerable synergy (peaks of 0.2-0.4 only; FIG. 17A). No considerable synergy was apparent for cisplatin with F5-SM. Rather, some inhibition of activity was apparent (FIG. 17A). However, F7-SM interacted synergistically with cisplatin (peaks at 0.2-0.4; FIG. 17A).

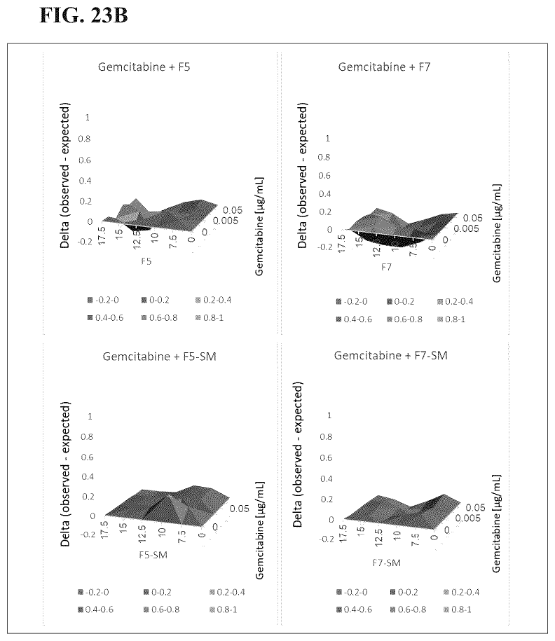

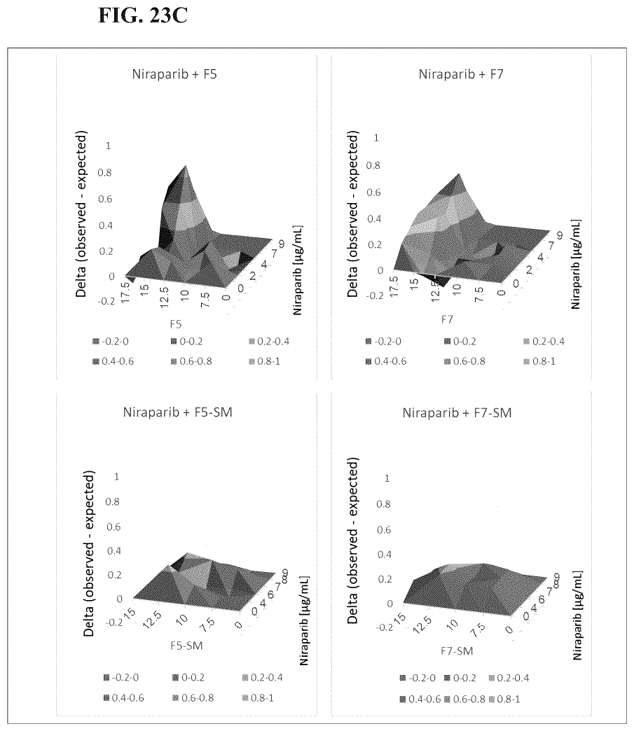

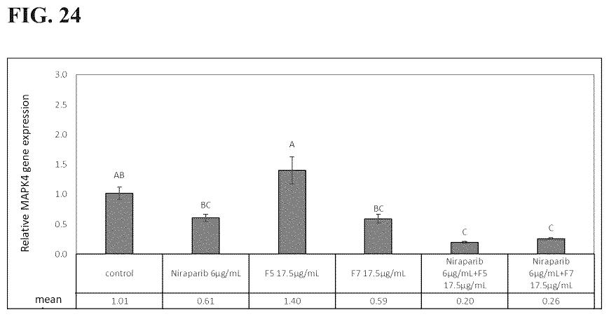

Gemcitabine on the other hand showed no considerable synergy with the plant fractions nor their corresponding SMs (FIG. 23B). Conversely, niraparib interacted synergistically with F5 and F7 (with peaks of 0.6-0.8 to 0.4-0.6, respectively). Synergy with F5-SM or F7-SM was restricted to 0.2-0.4 only (FIG. 23C).

### Example 16

### Determination of Expression of Markers in the Various Synergistic and Individual Treatments of Cannabis Fractions and Chemotherapy Drugs

The expression of several genes that were previously found to be differentially expression in OC was examined, following treatment with synergistic combinations of niraparib and F5 or F7, or cisplatin and F5 or F7.

Mitogen-activated protein kinase 4 (MAPK4) expression was reduced upon niraparib treatment and F7, but induced in the F5 treatment (FIGS. 18A-C). Significantly, the level of MAPK4 expression was substantially reduced in the synergistic treatment of niraparib+F5 or niraparib+F7 treatments.

### Example 17

### In Vivo Treatment with a Fraction According to Some Embodiments of the Present Invention as Well as Chemotherapy and Antibody Therapy

### Material and Methods—Mice Experiment

Female athymic nude mice (Balb/c) were used in xenograft model. The mice were obtained from Envigo Israel (6-8 weeks old) with initial body weight of 17-20 g and were maintained in pathogen-free conditions with free access to commercial chow and water in SIA facility (Nes Ziona, Israel). Animal experiment protocols were conducted in strict accordance with the Institutional Guidelines of Animal Care and Use Committee of Israel, authorization no. IL-2108-111-4.

Animals were inoculated subcutaneously with 2*10\hat{ }⁶ OVCAR3 (HTB-161) cells commonly used for OC-related xenograft studies (e.g., [1]) into the right flank in 200 ul solution contain 1:1 PBS/Matrigel (356237 Corning, Discovery Labware INC). When the tumor volume reached about 100 mm 3, mice were divided to 3 treatment groups, 8 mice per group. The following treatments were given: niraparib 25 mg/kg+avastin 5 mg/k; niraparib 25 mg/kg+avastin 5 mg/kg+F7 50 mg/kg; or vehicle control (10% v/v methanol in saline; 20 μl/mouse/treatment=1000 mg/kg; LD50 of methanol given orally per mouse ˜7300 mg/kg [2]. Treatments were given IP every other day and Avastin was given orally every 3 days. Tumor size was evaluated during the experiment using the formula V=½·(L·W²); (L and W are length and width of tumor, respectively). Postmortem Examination was done by Patho-Logica (Nes Ziona Israel): Tumors were excised (whole) from each animal and volume was measured. Tumors from the 24 mice were harvested, fixed in 4% formaldehyde and transferred to Patho-Logica (Israel) in the fixative. The organs were further fixed for 48 hrs and then trimmed and put in embedding cassettes. One cassette was prepared per animal. For slide preparation paraffin sections (4 microns thick), were cut, put on glass slides and stained with Hematoxylin & Eosin (H&E), using standard routine protocol, for general morphology. The slides were subjected to histopathological evaluation by Dr. Loeb (Patho Logica, Ness Ziona, Israel). Pictures were taken using Olympus microscope (BX60, serial NO. 7D04032) equipped with microscope's Camera (Olympus DP73, serial NO. OH05504) at objective magnification of X4.

### Results

Determining the Effect of Co-Treatment with Niraparib and F7 on Tumor Size In Vivo

Treatment of xenograft mice bearing HTB161-tumor with niraparib+avastin resulted in marked reduction in tumor growth in comparison to vehicle control (FIG. 25A, B). At the end of the experiment (14 days following the commence of treatments) niraparib+avastin treatment resulted in tumor at a size of ˜41 mm³, whereas in the vehicle control, the tumor continued to grow throughout the treatments period, to ˜135 mm 3 (FIG. 25A-B). Moreover, treatment with niraparib+avastin+F7 led to a further reduction in tumor size (30 mm 3 at the end of experiment; FIG. 25A-B). The further reduction in tumor size in the niraparib+avastin+F7 in comparison to niraparib+avastin treatment was significantly different at days 4 and 7 following the beginning of the treatments (FIG. 25A). There was a minor but significant reduction in body weight in the niraparib+avastin+F7 treated group, from day 2 of the treatment (FIG. 25C). Histological H&E staining of the obtained tumors demonstrated a marked change in the tumor's necrosis and amount of matrix available in control treat group vs. groups treated with avastin+niraparib and avastin+niraparib+F7 (FIG. 25D). In the control, large areas of neoplastic cells producing mucin (red arrow), and a central necrotic core (yellow arrow) were evident. Whereas in the treated tumors only remnants of neoplastic cells (red arrow), surrounded by vast area of necrotic tissue (yellow arrow) were revealed (FIG. 25D).

Hence, F7+niraparib showed improved anti-cancer activity also in vivo. Treatment of OC (HTB161) xenograft mice with niraparib+avastin+F7 resulted in a marked reduction in tumor size, of ˜4.5 fold in comparison to control. This niraparib+avastin+F7 combined treatment was significantly better than that of niraparib+avastin at 4 and 7 days post commence of treatment. In parallel, body weight of the treated mice was only slightly affected.

### REFERENCES

1. Zhang X, Jiang F, Li P, Li C, Ma Q, Nicosia S V, Bai W. Growth suppression of ovarian cancer xenografts in nude mice by vitamin D analogue EB1089. Clinical Cancer Research. 2005 Jan. 1; 11(1):323-8.
2. Clary J J. 4 General Animal and Aquatic Toxicity. The Toxicology of Methanol. 2013 Jan. 9:73

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Louis, D. N.; Ohgaki, H.; Wiestler, O. D.; Cavenee, W. K.; Burger, P. C.; Jouvet, A.; Scheithauer, B. W.; Kleihues, P., The 2007 WHO classification of tumours of the central nervous system. Acta Neuropathol. 2007, 114, 97-109.
2. Louis, D. N.; Perry, A.; Reifenberger, G.; Von Deimling, A.; Figarella-Branger, D.; Cavenee, W. K.; Ohgaki, H.; Wiestler, O. D.; Kleihues, P.; Ellison, D. W., The 2016 World Health Organization classification of tumors of the central nervous system: a summary. Acta Neuropathol. 2016, 131, 803-820.
3. Fernandes, C.; Costa, A.; Osório, L.; Lago, R. C.; Linhares, P.; Carvalho, B.; Caeiro, C., Current standards of care in glioblastoma therapy. Exon Publications 2017, 197-241.
4. Singh, S. K.; Clarke, I. D.; Terasaki, M.; Bonn, V. E.; Hawkins, C.; Squire, J.; Dirks, P. B., Identification of a cancer stem cell in human brain tumors. Cancer Res. 2003, 63, 5821-5828.
5. Lathia, J. D.; Mack, S. C.; Mulkearns-Hubert, E. E.; Valentim, C. L.; Rich, J. N., Cancer stem cells in glioblastoma. Genes Dev. 2015, 29, 1203-1217.
6. Perazzoli, G.; Prados, J.; Ortiz, R.; Caba, O.; Cabeza, L.; Berdasco, M.; Gónzalez, B.; Melguizo, C., Temozolomide resistance in glioblastoma cell lines: implication of MGMT, MMR, P-glycoprotein and CD133 expression. PloS One 2015, 10, e0140131.
7. Corroon, J.; Sexton, M.; Bradley, R., Indications and administration practices amongst medical cannabis healthcare providers: a cross-sectional survey. BMC Fam. Pract. 2019, 20, 1-12.
8. Hanuš, L. O.; Meyer, S. M.; Muñoz, E.; Taglialatela-Scafati, O.; Appendino, G. Phytocannabinoids: a unified critical inventory. Nat. Prod. Rep. 2016, 33, 1357-1392.
9. ElSohly, M. A.; Radwan, M. M.; Gul, W.; Chandra, S.; Galal, A., Phytochemistry of Cannabis sativa L. Phytocannabinoids 2017, 1-36.
10. Aizpurua-Olaizola, O.; Soydaner, U.; Öztürk, E.; Schibano, D.; Simsir, Y.; Navarro, P.; Etxebarria, N.; Usobiaga, A., Evolution of the cannabinoid and terpene content during the growth of Cannabis sativa plants from different chemotypes. J. Nat. Prod. 2016, 79, 324-331.
11. Ramer, R.; Hinz, B. Cannabinoids as anticancer drugs. In, Advances in Pharmacology 80, ed. by Kendall, D; S. P. H. Alexander (Elsevier Academic Press, 2017), 2017, pp. 397-436.
12. Baram, L.; Peled, E.; Berman, P.; Yellin, B.; Besser, E.; Benami, M.; Louria-Hayon, I.; Lewitus, G. M.; Meiri, D. The heterogeneity and complexity of Cannabis extracts as antitumor agents. Oncotarget 2019, 10, 4091.
13. Velasco, G.; Sánchez, C.; Guzmán, M., Towards the use of cannabinoids as antitumour agents. Nat. Rev. Cancer 2012, 12, 436-444.
14. Ellert-Miklaszewska, A.; Ciechomska, I. A.; Kaminska, B., Cannabinoid signaling in glioma cells. Glioma Signaling 2020, 223-241.
15. Dumitru, C. A.; Sandalcioglu, I. E.; Karsak, M., Cannabinoids in glioblastoma therapy: new applications for old drugs. Front. Mol. Neurosci. 2018, 11, 159.
16. Hernández-Tiedra, S.; Fabrias, G.; Dávila, D.; Salanueva, I. J.; Casas, J.; Montes, L. R.; Anton, Z.; Garcia-Taboada, E.; Salazar-Roa, M.; Lorente, M., Dihydroceramide accumulation mediates cytotoxic autophagy of cancer cells via autolysosome destabilization. Autophagy 2016, 12, 2213-2229.
17. Salazar, M.; Carracedo, A.; Salanueva, Í. J.; Hernandez-Tiedra, S.; Lorente, M.; Egia, A.; Vázquez, P.; Blázquez, C.; Torres, S.; García, S., Cannabinoid action induces autophagy-mediated cell death through stimulation of ER stress in human glioma cells. J. Clin. Invest. 2009, 119, 1359-1372.
18. Luís, Â.; Marcelino, H.; Rosa, C.; Domingues, F.; Pereira, L.; Cascalheira, J. F., The effects of cannabinoids on glioblastoma growth: A systematic review with meta-analysis of animal model studies. Eur. J. Pharmacol. 2020, 876, 173055.
19. Galanti, G.; Fisher, T.; Kventsel, I.; Shoham, J.; Gallily, R.; Mechoulam, R.; Lavie, G.; Amariglio, N.; Rechavi, G.; Toren, A., Δ9-Tetrahydrocannabinol inhibits cell cycle progression by downregulation of E2F1 in human glioblastoma multiforme cells. Acta Oncol. 2008, 47, 1062-1070.
20. Marcu, J. P.; Christian, R. T.; Lau, D.; Zielinski, A. J.; Horowitz, M. P.; Lee, J.; Pakdel, A.; Allison, J.; Limbad, C.; Moore, D. H., Cannabidiol enhances the inhibitory effects of Δ9-tetrahydrocannabinol on human glioblastoma cell proliferation and survival. Mol. Cancer Ther. 2010, 9, 180-189.
21. Solinas, M.; Massi, P.; Cinquina, V.; Valenti, M.; Bolognini, D.; Gariboldi, M.; Monti, E.; Rubino, T.; Parolaro, D., Cannabidiol, a non-psychoactive cannabinoid compound, inhibits proliferation and invasion in U87-MG and T98G glioma cells through a multitarget effect. PLoS One 2013, 8, e76918.
22. Lah, T. T.; Novak, M.; Pena Almidon, M. A.; Marinelli, O.; Žvar Bašcovič, B.; Majc, B.; Mlinar, M.; Bošnjak, R.; Breznik, B.; Zomer, R., Cannabigerol is a potential therapeutic agent in a novel combined therapy for glioblastoma. Cells 2021, 10, 340.
23. Guzman, M.; Duarte, M.; Blazquez, C.; Ravina, J.; Rosa, M.; Galve-Roperh, I.; Sánchez, C.; Velasco, G.; González-Feria, L., A pilot clinical study of Δ9-tetrahydrocannabinol in patients with recurrent glioblastoma multiforme. Br. J. Cancer 2006, 95, 197-203.
24. Schultz, S.; Beyer, M., GW pharmaceuticals achieves positive results in phase 2 proof of concept study in glioma. http://ir(dot)gwpharm(dot)com/static-files/cde942fe-555c-4b2f-9cc9-f34d24c7ad2, 2017.
25. Schultz, S. GW pharmaceuticals plc investor presentation. http://ir(dot)gwpharm(dot)com/static-files/e7afbad8-ab2c-4c8a-8e21-b9d3a7d36c70, 2018.
26. Russo, E., Taming THC: Potential cannabis synergy and phytocannabinoid-terpenoid entourage effects. Br. J. Pharmacol. 2011, 163, 1344-1364.
27. Russo, E. B., The case for the entourage effect and conventional breeding of clinical cannabis: no “strain,” no gain. Front Plant Sci. 2019, 9, 1969.
28. Koltai, H.; Namdar, D. Cannabis phytomolecule ‘entourage’: from domestication to medical use. Trends Plant Sci., 2020, 10, 976-984.
29. Mazuz, M.; Tiroler, A.; Moyal, L.; Hodak, E.; Nadarajan, S.; Vinayaka, A. C.; Gorovitz-Haris, B.; Lubin, I.; Drori, A.; Drori, G.; Van Cauwenberghe, O., Synergistic cytotoxic activity of cannabinoids from Cannabis sativa against cutaneous T-cell lymphoma (CTCL) in-vitro and ex-vivo. Oncotarget 2020, 11, 1141.
30. Jiang, W.; Finniss, S.; Cazacu, S.; Xiang, C.; Brodie, Z.; Mikkelsen, T.; Poisson, L.; Shackelford, D. B.; Brodie, C., Repurposing phenformin for the targeting of glioma stem cells and the treatment of glioblastoma. Oncotarget 2016, 7, 56456.
31. Giladi, N. D.; Ziv-Av, A.; Lee, H. K.; Finniss, S.; Cazacu, S.; Xiang, C.; Ben-Asher, H. W.; Decarvalho, A.; Mikkelsen, T.; Poisson, L., RTVP-1 promotes mesenchymal transformation of glioma via a STAT-3/IL-6-dependent positive feedback loop. Oncotarget 2015, 6, 22680.
32. Kazimirsky, G.; Jiang, W.; Slavin, S.; Ziv-Av, A.; Brodie, C., Mesenchymal stem cells enhance the oncolytic effect of Newcastle disease virus in glioma cells and glioma stem cells via the secretion of TRAIL. Stem Cell Res. Ther. 2016, 7, 1-10.
33. Singec, I.; Knoth, R.; Meyer, R. P.; Maciaczyk, J.; Volk, B.; Nikkhah, G.; Frotscher, M.; Snyder, E. Y., Defining the actual sensitivity and specificity of the neurosphere assay in stem cell biology. Nat. Methods 2006, 3, 801-806
34. Gopinath, S.; Vanamala, S. K.; Gujrati, M.; Klopfenstein, J. D.; Dinh, D. H.; Rao, J. S., Doxorubicin-mediated apoptosis in glioma cells requires NFAT3. Cell. Mol. Life Sci. 2009, 66, 3967-3978.
35. Namdar, D.; Anis, O.; Poulin, P.; Koltai, H., Chronological review and rational and future prospects of cannabis-based drug development. Molecules 2020, 25, 4821.
36. Tomko, A. M.; Whynot, E. G.; Ellis, L. D.; Dupré, D. J., Anti-cancer potential of cannabinoids, terpenes, and flavonoids present in cannabis. Cancers 2020, 12, 1985.
37. Hart, S.; Fischer, O. M.; Ullrich, A., Cannabinoids induce cancer cell proliferation via tumor necrosis factor α-converting enzyme (TACE/ADAM17)-mediated transactivation of the epidermal growth factor receptor. Cancer Res. 2004, 64, 1943-1950.
38. Vermeulen, K.; Berneman, Z. N.; Van Bockstaele, D. R., Cell cycle and apoptosis. Cell Prolif 2003, 36, 165-175.
39. Iurlaro, R.; Muñoz-Pinedo, C., Cell death induced by endoplasmic reticulum stress. FEBS J. 2016, 283, 2640-2652.
40. Han, J.; Back, S. H.; Hur, J.; Lin, Y.-H.; Gildersleeve, R.; Shan, J.; Yuan, C. L.; Krokowski, D.; Wang, S.; Hatzoglou, M., ER-stress-induced transcriptional regulation increases protein synthesis leading to cell death. Nat. Cell Biol. 2013, 15, 481-490.
41. Fang, N.; Zhang, W.; Xu, S.; Lin, H.; Wang, Z.; Liu, H.; Fang, Q.; Li, C.; Peng, L.; Lou, J., TRIB3 alters endoplasmic reticulum stress-induced β-cell apoptosis via the NF-κB pathway. Metabolism 2014, 63, 822-830.
42. Salazar, M.; Carracedo, A.; Salanueva, Í. J.; Hernández-Tiedra, S.; Egia, A.; Lorente, M.; Vázquez, P.; Torres, S.; Iovanna, J. L.; Guzmán, M., TRB3 links ER stress to autophagy in cannabinoid antitumoral action. Autophagy 2009, 5, 1048-1049.
43. Ohoka, N.; Yoshii, S.; Hattori, T.; Onozaki, K.; Hayashi, H., TRB3, a novel ER stress-inducible gene, is induced via ATF4-CHOP pathway and is involved in cell death. EMBO J. 2005, 24, 1243-1255.
44. Borrelli, F.; Pagano, E.; Romano, B.; Panzera, S.; Maiello, F.; Coppola, D.; De Petrocellis, L.; Buono, L.; Orlando, P.; Izzo, A. A., Colon carcinogenesis is inhibited by the TRPM8 antagonist cannabigerol, a Cannabis-derived non-psychotropic cannabinoid. Carcinogenesis 2014, 35, 2787-2797.
45. Izzo, A. A.; Borrelli, F.; Capasso, R.; Di Marzo, V.; Mechoulam, R., Non-psychotropic plant cannabinoids: new therapeutic opportunities from an ancient herb. Trends Pharmacol. Sci. 2009, 30, 515-527.
46. New, D. C.; Wong, Y. H., BML-190 and AM251 act as inverse agonists at the human cannabinoid CB2 receptor: signalling via cAMP and inositol phosphates. FEBS Lett. 2003, 536, 157-160.
47. Olson, M. F.; Sahai, E., The actin cytoskeleton in cancer cell motility. Clin. Exp. Metastasis 2009, 26, 273.
48. Memmel, S.; Sisario, D.; Zöller, C.; Fiedler, V.; Katzer, A.; Heiden, R.; Becker, N.; Eing, L.; Ferreira, F. L.; Zimmermann, H., Migration pattern, actin cytoskeleton organization and response to PI3K-, mTOR-, and Hsp90-inhibition of glioblastoma cells with different invasive capacities. Oncotarget 2017, 8, 45298.
49. Jacquemet, G.; Hamidi, H.; Ivaska, J., Filopodia in cell adhesion, 3D migration and cancer cell invasion. Curr. Opin. Cell Biol. 2015, 36, 23-31.
50. Lukas, R. V.; Wainwright, D. A.; Ladomersky, E.; Sachdev, S.; Sonabend, A. M.; Stupp, R., Newly diagnosed glioblastoma: a review on clinical management. Oncology 2019, 33, 91.
51. Bush, N. A. O.; Chang, S. M.; Berger, M. S., Current and future strategies for treatment of glioma. Neurosurg. Rev. 2017, 40, 1-14.
52. Yuan, X.; Curtin, J.; Xiong, Y.; Liu, G.; Waschsmann-Hogiu, S.; Farkas, D. L.; Black, K. L.; John, S. Y., Isolation of cancer stem cells from adult glioblastoma multiforme. Oncogene 2004, 23, 9392-9400.
53. Hong, X.; Chedid, K.; Kalkanis, S. N., Glioblastoma cell line-derived spheres in serum-containing medium versus serum-free medium: A comparison of cancer stem cell properties. Int J Oncol 2012, 41, 1693-1700.
54. Kataoka, J. M., R. Patel, D J Grdina, Y, Cytoprotection by WR-1065, the active form of amifostine, is independent of p53 status in human malignant glioma cell lines. Int. J. Radiat. Biol. 2000, 76, 633-639.
55. Altinoz, M. A.; Ucal, Y.; Yilmaz, M. C.; Kiris, İ.; Ozisik, O.; Sezerman, U.; Ozpinar, A.; Elmaci, İ., Progesterone at high doses reduces the growth of U87 and A172 glioblastoma cells: Proteomic changes regarding metabolism and immunity. Cancer Med. 2020, 9, 5767-5780.
56. Gallaher, J. A.; Massey, S. C.; Hawkins-Daarud, A.; Noticewala, S. S.; Rockne, R. C.; Johnston, S. K.; Gonzalez-Cuyar, L.; Juliano, J.; Gil, O.; Swanson, K. R., From cells to tissue: How cell scale heterogeneity impacts glioblastoma growth and treatment response. PLoS Comput. Biol. 2020, 16, e1007672.

What is claimed is:

1. A method of treating cancer in a subject in need thereof, the method comprising administering to the subject a therapeutically effective amount of a composition selected from the group consisting of:
(i) a composition comprising phytocannabinoids, wherein said phytocannabinoids comprise at least 70% cannabigerol (CBG) and at least one phytocannabinoid selected from the group consisting of cannabinol (CBN) and tetrahydrocannabivarin (THCV);
(ii) a composition comprising phytocannabinoids, wherein said phytocannabinoids comprise at least 85% tetrahydrocannabinol (THC) and at least 1.5% cannabigerol (CBG);
(iii) a composition comprising phytocannabinoids, wherein said phytocannabinoids comprise at least 50% tetrahydrocannabinol (THC), at least 10% cannabichromene (CBC) and at least one phytocannabinoid selected from the group consisting of cannabidivarin (CBDV), cannabidivarinic acid (CBDVA), cannabigerol (CBG), cannabinol (CBN) and tetrahydrocannabinolic acid (THCA); and
(iv) a composition comprising phytocannabinoids, wherein said phytocannabinoids comprise at least 80% tetrahydrocannabinol (THC) and at least three phytocannabinoids selected from the group consisting of cannabichromene (CBC), cannabigerol (CBG), cannabinol (CBN), tetrahydrocannabinolic acid (THCA) and tetrahydrocannabivarin (THCV),
thereby treating the cancer in the subject.

2. A composition selected from the group consisting of:
(i) a composition comprising the phytocannabinoids listed in the F4 composition of Table 1 in percentages as listed in the F4 composition of Table 1±10%;
(ii) a composition comprising the phytocannabinoids listed in the F5 composition of Table 1 in percentages as listed in the F5 composition of Table 1±10% or a composition comprising the phytocannabinoids listed in the F6 composition of Table 1 in percentages as listed in the F6 composition of Table 1±10%;
(iii) a composition comprising the phytocannabinoids listed in the F7 composition of Table 1 in percentages as listed in the F7 composition of Table 1±10%;
(iv) a composition comprising the phytocannabinoids listed in the crude extract composition of Table 1 in percentages as listed in the crude extract composition of Table 1±10%;
(v) a composition comprising the phytocannabinoids listed in the sCBD crude extract composition of Table 7 in percentages as listed in the crude extract composition of Table 7±10%; and
(vi) a composition comprising the phytocannabinoids listed in the PARIS crude extract composition of Table 7 in percentages as listed in the crude extract composition of Table 7±10%.

3. A method of treating cancer in a subject in need thereof, the method comprising administering to the subject a therapeutically effective amount of the composition of claim 2, thereby treating the cancer in the subject.

4. A method of reducing viability, inducing cell cycle arrest and/or reducing proliferation and/or migration of a cancerous cell, the method comprising contacting the cancerous cell with the composition of claim 2.

5. The method of claim 1, further comprising administering to the subject a therapeutically effective amount of an anti-cancer agent.

6. The method of claim 4, further comprising contacting said cancerous cell with an anti-cancer agent.

7. An article of manufacture comprising an anti-cancer agent and a composition selected form the group consisting of:
(i) a composition comprising phytocannabinoids, wherein said phytocannabinoids comprise at least 70% cannabigerol (CBG) and at least one phytocannabinoid selected from the group consisting of cannabinol (CBN) and tetrahydrocannabivarin (THCV);
(ii) a composition comprising phytocannabinoids, wherein said phytocannabinoids comprise at least 85% tetrahydrocannabinol (THC) and at least 1.5% cannabigerol (CBG);
(iii) a composition comprising phytocannabinoids, wherein said phytocannabinoids comprise at least 50% tetrahydrocannabinol (THC), at least 10% cannabichromene (CBC) and at least one phytocannabinoid selected from the group consisting of cannabidivarin (CBDV), cannabidivarinic acid (CBDVA), cannabigerol (CBG), cannabinol (CBN) and tetrahydrocannabinolic acid (THCA); and
(iv) a composition comprising phytocannabinoids, wherein said phytocannabinoids comprise at least 80% tetrahydrocannabinol (THC) and at least three phytocannabinoids selected from the group consisting of cannabichromene (CBC), cannabigerol (CBG), cannabinol (CBN), tetrahydrocannabinolic acid (THCA) and tetrahydrocannabivarin (THCV).

8. An article of manufacture comprising an anti-cancer agent and the composition of claim 2.

9. The article of manufacture of claim 7, wherein said anti-cancer agent and said composition are provided in a co-formulation.

10. The article of manufacture of claim 7, wherein said anti-cancer agent and said composition are provided in separate formulations.

11. The method of claim 5, wherein said anti-cancer agent comprises a chemotherapy.

12. The method of claim 11, wherein said chemotherapy is selected from the group consisting of cisplatin and niraparib and optionally wherein the composition is as claimed in claim 1(ii) or 1(iii).

13. The method of claim 1, wherein said composition has anti-cancer activity on Glioblastoma multiforme (GMB) cells and/or ovarian cancer cells.

14. The method of claim 1, wherein said composition has a combined synergistic anti-cancer activity on glioblastoma multiforme (GMB) cells and/or ovarian cancer cells as compared to each of said phytocannabinoids comprised in said composition when administered as a single agent.

15. The method of claim 1, wherein said cancer is glioblastoma.

16. The method of claim 15, wherein said cancer is Glioblastoma multiforme (GMB).

17. The method of claim 1, wherein said cancer is ovarian cancer.

18. The method of claim 17, wherein said composition of as listed in claim 1(ii) or 1(iii).

19. The method of claim 1, wherein said composition (i) comprises the phytocannabinoids listed in the F4 composition of Table 1.

20. The method of claim 1, wherein said composition (ii) comprises the phytocannabinoids listed in the F5 composition of Table 1.
